(19) United States
(12) Patent Application Publication
(10) Pub. No.: US 2004/0059242 A1
(43) Pub. Date: Mar. 25, 2004

(54) Body composition measurement method and apparatus

(76) Inventors: Yoshihisa Masuo, Kyoto (JP); Kazuhiko Yoshida, Kyoto (JP)

Correspondence Address: Oliff & Berridge, PO Box 19928, Alexandria, VA, 22320, US

(21) Appl. No.: 10/432,666

(22) PCT Filed: Nov. 28, 2001
(86) PCT No.: PCT/JP01/10420

(30) Foreign Application Priority Data
Nov. 29, 2000 (JP) 2000-623896
Dec. 26, 2000 (JP) 2000-394491

(51) Int. Cl. A61B 5/05

(52) U.S. Cl. 600/547

(57) ABSTRACT

To accurately measure the muscle mass, body-fat ratio and/or other information of the subject, the human body is divided into nine segments including the trunk, right and left forearms, right and left upper arms, right and left thigh, and right and left crura. Four distal voltage-measuring points Pv are determined at the wrists and ankles, and four proximal voltage-measuring points Pv are determined at the elbows and knees. Four current-carrying electrodes and four measuring electrodes are used. First, the measuring electrodes are attached to distal points, and the impedances of four limbs and the trunk are measured. Then, the measuring electrodes are moved to proximal points, and the impedances of four limbs and the trunk are measured. From the measurement result, the measurement value is calculated for each segment. Then, using estimation formulae created by a regression analysis based on the data collected with an MRI, the body composition such as a muscle mass is estimated from the measurement values of the impedances and body specific information including the height, weight, etc.

### TECHNICAL FIELD

The present invention relates to a body composition measurement method and apparatus for measuring bioelectrical impedance of the body of a subject to estimate and present various kinds of information about body composition (such as body-fat mass, muscles, bone mass, bone density, lean body mass, body-fat ratio and basal metabolic rate), health condition and physical capability of the subject, using not only the measurement value of the impedance but also the height, weight, age, gender and other information, which will be generally referred to as “body specific information” hereinafter.

### BACKGROUND ART

Conventionally, the most common method in health management concerning obesity or other body conditions is to measure body weight. Nowadays, obesity is not regarded simply as a body type, but people are looking at indices for measuring obesity. One such index is the body-fat mass, which indicates the mass of subcutaneous fat and/or visceral fat; another is the body-fat ratio, which indicates the ratio of body-fat to body weight.

Many parties have been studying methods of measuring the bioelectrical impedance of the body (simply referred to as the “impedance” hereinafter) and estimating body-fat ratio and/or other values based on the impedance. One such method is a four-electrode method, which uses a pair of current-carrying electrodes attached to the back of the right hand and the instep of the right foot of the subject, respectively, and a pair of measuring electrodes attached to some parts between the current-carrying electrodes, such as the right wrist and the right ankle. With a radio-frequency current being supplied through the body between the current-carrying electrodes, the potential difference between the measuring electrodes is measured. Then, the impedance is calculated from the voltage value and the current value, and the body-fat ratio and/or other information are estimated based on the measurement value of the impedance.

Recently, more convenient apparatuses for measuring body-fat ratio (so-called “body-fat meters”) have been developed, and are commercially available. An example of such apparatuses is disclosed in the Japanese Unexamined Patent Publication No. H7-51242. This apparatus includes a pair of grips to be held with both hands, each grip provided with a current-carrying electrode and a measuring electrode. The electrodes are located so that, when the subject holds the grips with both hands, the current-carrying electrode contacts the finger-side part of the hand and the measuring electrode contacts the wrist-side part of the hand. Then, based on the bioelectrical impedance measured thereby, the apparatus estimates various kinds of information, such as lean body mass, body-fat ratio, total body water or basal metabolic rate. Another example is disclosed in the Japanese Examined Patent Publication No. H5-49050. This apparatus has a measuring platform, on which the above electrodes are located to contact the soles when the subject steps on it, so that the weight and the body-fat can be measured simultaneously.

The above-described apparatuses measure the impedance with the current path extending between one hand and one foot, between both hands or between both legs. In the case of measuring the voltage with the current path between one hand and one foot, the current path includes the chest or abdomen (i.e. trunk), whose cross-sectional area is much larger than that of the leg or the arm. In this case, the contribution of the leg or the arm to the impedance is relatively great while that of the subcutaneous fat of the abdomen or the intra-abdominal fat (visceral fat) is relatively small. This means that the measurement result hardly reflects the increase or decrease in the subcutaneous fat of the abdomen or the intra-abdominal fat, so that the result lacks reliability. In the case of measuring the voltage with the current path between both hands or both feet, on the other hand, most of the trunk is excluded from the current path, so that the error in the estimation of the body-fat ratio or other indices for the entire body is likely to be large.

Conventionally, when the body-fat ratio or other indices are estimated from the measurement value of the impedance, an estimation formula for the bioelectrical impedance approach (BIA), created according to a calibration curve prepared using the underwater weighing method as the estimation basis, is used. By this method, however, it is impossible to decrease the estimation error because the method has some faults, such as lack of consideration of the difference in the contribution of the muscle, bone or other lean body components to the impedance.

The calculation of body composition from the impedance is based on the assumption that the human body can be modeled as a structure composed of three tissues: bone, muscle and fat, which have different electrical characteristics. In this model, it is assumed that the three tissues are connected in parallel, the component ratios of the tissues are fixed, and the whole component tissues and each tissue have fixed electrical characteristics (i.e. volume resistivity). Various statistical researches suggest that this assumption is highly reliable for normal adults. However, for children, minors, elderly people, athletes or other groups of people having special body composition, it is impractical to obtain reliable results because there are personal differences in the component ratios and electrical characteristics, which often make the aforementioned values deviate from the above condition.

In respect not of the prevention of obesity, but in respect of the determination of the progress of strengthening of the body or the progress of aging, it is very important to measure the muscle mass or muscle force of the body. For example, an athlete or similar person intending to improve the ability of the body will not only regard the muscle mass as an index for measuring the effect of training or other activities but also use the index to set an objective for training. This observation applies also for the case of a person in the process of rehabilitation for the strengthening and recovery of a part of the body that has weakened after a long hospital stay due to an injury or illness. Furthermore, the expected increase of elderly people will make it necessary to easily measure the muscle mass, muscle force and right-and-left balance of such properties of muscles for each elderly person in nursing care. The information obtained by the measurement will make it possible to evaluate the ability of the person to live an independent life, and to provide an improved living environment and diet plan (meals, exercises) that are designed to supplement any inconvenience or shortage in daily life so that the person can exhibit a high performance in daily activity.

Conventional apparatuses in this field cannot provide aforementioned kinds of information or can merely provide such information with inadequate accuracy.

It is of course possible to perform accurate measurements by using a magnetic resonance imaging apparatus or an X-ray computed tomography scanner, which are generally used in large hospitals. These apparatuses, however, occupy large spaces and are very costly. Furthermore, the apparatuses keep the subject in the bound state and impose physical and mental burdens on the subject, regardless of their age.

Preferably, the apparatus should be small enough for welfare workers or similar persons to take it with them when visiting elderly people, and easy enough to perform measurements on the subjects at their homes. In other words, it is desirable that well-trained persons can easily perform the measurement on the subject with the apparatus. It is also desirable that the apparatus does not require extraordinary production costs. When all these conditions are met, the apparatus will be extremely valuable, even if it is not easy for everybody to use.

Furthermore, the apparatus should be preferably as easy to use as height scales, weighing machines or other conventional apparatuses generally used for measuring body sizes. With such an apparatus, the measurement can be conveniently performed as a part of health examination, for example. In addition, the apparatus should be produced at such a low price that allows us to personally purchase and use it in our everyday life to maintain and promote our health.

The present invention has been accomplished in view of the above problems, the first object of which is to propose a body composition measurement method and apparatus that is relatively easy to use, inexpensive and capable of measuring the body-fat, muscle mass, muscle force, bone mass and/or other body composition information more accurately than conventional methods and apparatuses.

The second object of the present invention is to propose a body composition measurement method and apparatus capable of accurately measuring the body composition information of children, elderly people, athletes or other subjects whose body composition often differs greatly from that of normal adults.

The third object of the present invention is to propose a body composition measurement method and apparatus capable of providing an ADL (activity of daily living) index or other appropriate information to elderly people, trainees of rehabilitation, athletes or other people for whom it is especially helpful to obtain information about particular body compositions, such as muscle mass or muscle force, and the balance of such body compositions.

### DISCLOSURE OF THE INVENTION

To solve the above problems, the present invention proposes, as a first invention, a body composition measurement method, in which an impedance of the body of a subject is measured and information about a body composition and/or health condition of the subject is estimated based on the measurement value of the impedance, or based on the measurement value of the impedance and body specific information, wherein:

the entire human body is represented by a model in which the human body is divided into plural body parts, where the impedance of each body part can be approximately represented by a model composed of impedances respectively corresponding to at least fatty tissue, muscular tissue and osseous tissue connected in parallel, and component ratios of the tissues and electrical characteristics of the whole component tissues and each tissue can be regarded as fixed,

an alternating current is supplied between two current-carrying electrodes attached to the surface of the body at positions located outer than both ends of a target body part selected from the plural body parts so that the alternating current flows through at least the target body part,

the potential difference generated by the alternating current between both ends of the target body part is measured with two measuring electrodes attached to the surface of the body in the vicinity of the aforementioned ends or at positions lying not on the path of the alternating current but on paths each extending from the aforementioned ends, and

the impedance corresponding to the target body part is derived from the measurement value of the potential difference and the current value, and information relating to the body composition and/or health condition of the target body part or the entire body of the subject is estimated from the value of the impedance, or from the value and the body specific information.

Here, the “body parts, where the impedance of each body part can be approximately represented by a model composed of impedances respectively corresponding to at least fatty tissue, muscular tissue and osseous tissue connected in parallel, and component ratios of the tissues and electrical characteristics of the whole component tissues and each tissue can be regarded as fixed” are parts of the body, each of which can be approximately modeled as a column having a certain length and composed of tissues with their cross-sectional area ratios almost fixed. Examples of the body parts are as follows: “arm”, extending from wrist to shoulder (or acromion); “leg”, extending from ankle to groin (or trochanterion); and trunk (or idiosoma).

The “arm” may be further divided at the elbow into forearm and upper arm. Similarly, the “leg” may be divided at the knee into crus (or lower thigh) and thigh. In regard to the part of the upper limb excluded from the “arm”, i.e. the hand, the part between the wrist and the roots of the fingers at the back of the hand may be chosen as a body part (called “wrist area” hereinafter). Similarly, in regard to the lower limb, the part between the ankle and the roots of the fingers at the instep of the foot may be chosen as a body part (called “ankle area” hereinafter). It is possible to divide any of the aforementioned parts into smaller parts. For example, a part of the right or left forearm close to the wrist area, or a part of the crus close to the ankle area, may be chosen as a body part.

The “body specific information” typically includes the following information about the subject: height, weight, age and gender. Partial sizes of a body part, such as the length and/or circumference of a leg, may be also used. Furthermore, the history of illness and/or injury, or other information that influences the body and health, may be included.

The “information relating to the body composition and health condition” are, for example, the body-fat mass (or ratio), lean body mass (or ratio), total body water (or body water ratio), bone mass (or ratio), bone density, muscle force, degree of obesity, basal metabolic rate, energy metabolism, and ADL index for measuring the activity of daily life (or living) of the subject. The above quantities and ratios may be calculated for the entire body, each part of the body, or both.

In the body composition measurement method according to the first invention, the human body is divided into small plural body parts, and the impedance is obtained for each body part. The impedance of each body part can be approximately represented by a model composed of impedances respectively corresponding to at least fatty tissue, muscular tissue and osseous tissue connected in parallel, and component ratios of the tissues and electrical characteristics of the whole component tissues and each tissue can be regarded as fixed. Each body part resulting from the division closely coincides with the model used as a standard for the calculation of body composition. Therefore, the body composition information and/or other information of each body part can be accurately estimated from the impedance. Also, in regard to the estimation for the entire body, the body composition information and/or other information can be estimated more accurately than in the case of conventional methods.

The trunk may be represented by a model including five impedance elements: central part of the trunk; right and left shoulders connecting the upper ends of the right and left arms and the upper end of the central part of the trunk; and right and left groins connecting the upper ends of the right and left legs and the lower end of the central part of the trunk, and the impedance of the right or left shoulder, or right or left groin, may be estimated from at least an impedance corresponding to one of the plural body parts. By such a method, the impedances of the shoulders and groins, which are the components of the trunk, can be accurately estimated without directly attaching the measuring electrodes to the trunk.

The information relating to the body composition and/or health condition of the subject may be estimated from the impedances corresponding to the trunk and at least one of the plural body parts other than the trunk. This makes it possible to correct any particular imbalance relating to the bone, muscle, fat and so on, within the body, to improve the accuracy of the measurement.

The body composition measurement method according to the first invention may preferably use an estimation formula created based on the result of the measurement of the impedance for the entire body and/or each body part of each of plural pretest subjects, and based on the body composition criteria information of the entire body and/or each body part of each of the pretest subjects measured and collected with an apparatus capable of acquiring cross-sectional images, or created by further including additional body specific information of the pretest subjects, in order to estimate the information relating to the body composition and/or health condition based on the measurement value of the impedance of each body part of the subject, or based on the aforementioned value and the body specific information.

Examples of the “apparatus capable of acquiring cross-sectional images” are nuclear magnetic resonance imaging (MRI) apparatuses and computed tomography (CT) scanners. For example, MRIs can take cross-sectional images of the abdominal cavity, arms, legs or other body parts at preset intervals. From these cross-sectional images, the masses and occupation ratios of the living body tissues (fat, muscle, bone, etc.) in a given part of the body can be obtained by identifying each living body tissue in every cross-sectional image, calculating the mass and occupation ratio of each tissue, and integrating the results of the analysis of all the cross-sections included in the given part. The accuracy of the estimation formula can be greatly improved by performing the above measurement on a number of pretest subjects (or monitors) of different height, weight, age and gender (i.e. the body specific information), measuring the impedance corresponding to each body part of each pretest subject, and creating an estimation formula based on the measurement results. This method makes it possible to accurately estimate the information relating to the body composition and health condition of an unknown subject.

In the body composition measurement method according to the first invention, it is possible to measure the impedance of only one body part of the plural target body parts constituting the body, and derive the body composition information from the measurement value, or from the measurement value and the body specific information. More preferably, however, the body composition information should be derived from at least an significant measurement value of the impedances corresponding to all the body parts included in the target body parts, or from the significant measurement value and the body specific information. The “significant measurement value” is any measurement value that can affect the result of the statistic analysis, i.e. regression analysis, used in the present invention,. By this method, the body composition information is obtained with higher accuracy. Furthermore, the body composition information can be accurately obtained even for such a subject whose body is unbalanced in body composition between right and left, upper and lower, or distal and proximal parts of the body, or is extraordinarily developed at a particular part.

To solve the above problems, the present invention proposes, as a second invention and an embodiment of the body composition measurement method according to the first invention, a body composition measurement apparatus including a measuring means for measuring the impedance of the body of a subject, and an estimating means for estimating information relating to the body composition and/or health condition of the subject based on the measurement value of the impedance or based on the measurement value and body specific information, wherein:

the entire human body is represented by a model in which the human body is divided into plural body parts, where the impedance of each body part can be approximately represented by a model composed of impedances respectively corresponding to at least fatty tissue, muscular tissue and osseous tissue connected in parallel, and component ratios of the tissues and electrical characteristics of the whole component tissues and each tissue can be regarded as fixed,

and the measuring means include:

a) a current-generating means for generating an alternating current at a preset frequency;

b) at least two current-carrying electrodes to be attached to the surface of the body at positions outer than both ends of a target body part selected from the plural body parts to supply the alternating current through at least the target body part;

c) a voltage-measuring means, including at least two measuring electrodes to be attached to the surface of the body in the vicinity of the aforementioned ends of the target body part or at positions lying not on the path of the alternating current but on paths each extending from the aforementioned ends, for measuring the potential difference generated between the aforementioned ends of the target body part by the alternating current supplied from the current-carrying electrodes; and

d) a calculating means for calculating the impedance corresponding to the target body part from the measurement value of the potential difference and the current value of the alternating current,

and the estimating means estimates the information relating to the body composition and/or health condition of the target body part or the entire body of the subject based on the value of the impedance calculated by the calculating means, or based on the value and the body specific information.

The body composition measurement apparatus according to the second invention supplies a weak alternating current through the current-carrying electrodes into at least one target body part, and measures the voltage generated in the current path due to the impedance of the target body part with the voltage-measuring means via the measuring electrodes. Here, well-known four-electrode methods can be used. Even when the attachment positions for the electrodes are limited, for example when it is preferable to avoid attaching the electrodes to the trunk, the voltage corresponding to the voltage between both ends of the target body part can be measured as follows without problem. That is, no potential difference is produced along the voltage measurement path within such parts of the body where the above electric current does not flow, so that such parts can be regarded as mere lead wires as far as the measurement of the voltage is concerned. For example, when the current is supplied between the backs (or fingertips) of the hands, the right and left legs and the trunk can be regarded as mere lead wires. There, the voltage measured between the right wrist and the right ankle (or left ankle) can be regarded as equal to the voltage drop due to the impedance of the right arm, because the voltage measurement path overlaps the current path only at the right arm.

Thus, by appropriately selecting the attachment positions for the current-carrying electrodes and the measuring electrodes, it is possible to obtain the voltage drop across any body part of the subject, and the impedance corresponding to the body part can be derived from the measurement value of the voltage and the current value. Thus, the body composition measurement apparatus according to the second invention can provide accurate information relating to the composition of the entire body and the health condition as well as the composition of each body part. The frequency and magnitude of the current should be maintained constant throughout the measurement of a given body part, which, however, may be differently determined for different target body parts.

In a mode of the body composition measurement apparatus according to the second invention, attachment points for the measuring electrodes include at least four points in the vicinity of the right and left wrists and the right and left ankles. This construction makes it possible to divide the body of the subject into five segments corresponding to the right and left arms, the right and left legs and the trunk, and to obtain the impedance of each segment.

In addition to the aforementioned four positions, the attachment points for the measuring electrodes may further include at least one of four points in the vicinity of the right and left elbows and the right and left knees. For example, inclusion of all of these four points makes it possible to divide the body of the subject into nine segments corresponding to the right and left upper arms, the right and left forearms, the right and left thighs, the right and left crura and the trunk, and to obtain the impedance of each segment.

The attachment points for the measuring electrodes may further include at least one of four points located at the palms or backs of the right and left hands, and at the soles or insteps of the right and left feet. For example, inclusion of all of these four points in addition to the aforementioned four points makes it possible to divide the body of the subject into thirteen segments corresponding to the right and left upper arms, the right and left forearms, the right and left wrists, the right and left thighs, the right and left crura, the right and left ankles and the trunk, and to obtain the impedance of each segment.

The attachment points for the measuring electrodes may further include at least one of four points in the vicinity of the roots of the right and left arms and the roots of the right and left legs. This construction makes it possible to measure the voltages at the connection points between the trunk and the upper and lower limbs. Use of these voltages improves the accuracy of the impedance of each body part included in the upper and lower limbs, such as the upper arm or forearm, particularly the right and left upper arms and thighs. In addition, the impedance of the roots of the right and left arms or the right and left legs, which have been treated as parts of the trunk, can be accurately estimated.

The attachment points for the measuring electrodes may further include a part of the arm close to the wrist area or a part of the crus close to the ankle. In these parts, the occupation ratio of the osseous tissue in cross-sectional area is relatively high. Accordingly, these parts are especially suitable for obtaining accurate information about osseous tissue, such as bone mass or bone density.

The attachment points for the current-carrying electrodes may be four points located between the wrist and fingertips of the right and left hands, and between the ankle and fingertips of the right and left feet. When the measuring electrodes are also attached to the wrists and the ankles, the current-carrying electrodes should not be too close to those electrodes. In this case, the current-carrying electrodes may be preferably attached to the backs of the hands or the insteps of the feet in the vicinity of the roots of the fingers, or to the fingers. It is further preferable that the attachment points for the current-carrying electrodes include a finger or fingers of the hand or the feet, and the current-carrying electrodes are designed to be fixedly held between fingers or around a finger. This makes the electrode harder to come off than the electrode designed to be affixed on the palm or back of the hand, so that the measurement work can be efficiently performed.

In body composition measurement apparatus according to the second invention, the body may be divided into five segments corresponding to the right and left arms, the right and left legs and the trunk, where the segments corresponding to the arms and the legs are each modeled as one impedance element, whereas the trunk is represented by a model including five impedance elements: central part of the trunk; right and left shoulders connecting the upper ends of the right and left arms and the upper end of the central part of the trunk; and right and left groins connecting the upper ends of the right and left legs and the lower end of the central part of the trunk, and the estimating means estimates the impedances of the right and left shoulders and the right and left groins based on the impedance of at least one of the plural body parts of the subject.

This construction makes it possible to use the impedances corresponding to the right and left shoulders and the right and left groins to correct the measurement value of the impedances of other segments. This improves the accuracy of the measurement values, from which the body composition information or other information can be estimated more accurately.

In a mode of the second invention, the body composition measurement apparatus includes four current-carrying electrodes, four measuring electrodes and a current-carrying electrode selecting means for selectively supplying the alternating current between two of the four current-carrying electrodes, the voltage-measuring means selects two of the four measuring electrodes and measures the potential difference between the two measuring electrodes, the measuring electrodes are attached to the body at four points in the vicinity of the right and left wrist and the right and left ankles, or in the vicinity of the right and left elbows and the right and left knees, and the current-carrying electrodes are attached to the body at four positions located between the wrist and fingertips of the right and left hands, and between the ankle and fingertips of the right and left feet.

The above construction eliminates the necessity of changing the attachment positions of the current-carrying electrodes and the measuring electrodes during the measurement of the impedances of five segments including the right and left arms, the right and left legs and the trunk, or five segments including the right and left upper arms, the right and left thighs and the trunk. This reduces the workload of the examiner, and prevents mistaken changing of the attachment positions.

Suppose the measurement should be performed for a greater number of body parts. For example, suppose the impedances corresponding to the aforementioned nine segments should be measured. Use of additional measuring electrodes to be attached to the additional attachment points would not only require an increased number of electrodes but also make the wiring work very troublesome. In such a case, it is preferable to change the attachment positions of the four measuring electrodes from four points in the vicinity of the right and left wrists and the right and left ankles to four points in the vicinity of the right and left elbows and the right and left knees, or vice versa, and to measure the impedance of a predetermined body part for each arrangement of the attachment positions. This method can be similarly applied to other attachment positions. That is, it is possible to sequentially measure the impedances of desired body parts while changing the attachment positions of the measuring electrodes within the attachment points. This method is also applicable to the case of using two current-carrying electrodes and two measuring electrodes. This method requires only a small number of measuring electrodes, which makes the apparatus low-priced, the wiring less complex, the cables free from entanglement, and the mistaken attachment of electrodes by the examiner less probable.

In the case of changing the attachment positions of the measuring electrodes as described above, it is preferable to provide a measure for preventing mistaken positioning of the electrodes. Taking this into account, the body composition measurement apparatus according to the second invention may include a work-guiding means for indicating the attachment positions for the electrodes on the body of the subject, using at least one of image information, character information and voice information. With this construction, the examiner follows the instructions of the work-guiding means to attach the measuring electrodes to the body. This eliminates the mistaken attachment of the electrodes, so that the measurement can be correctly performed without unnecessary work.

For example, the work-guiding means may include an image-displaying means for showing markers on a figurative model of the human body to indicate the positions at which the measuring electrodes should be attached, and a display-controlling means for controlling the image-displaying means so that the markers are moved to new positions at which the measuring electrodes should be attached next after the completion of the measurement performed with the measuring electrodes attached at preset positions. This construction makes the mistaken work less probable because the attachment positions can be instantly recognized. It is of course possible to construct the image-displaying means to show the attachment positions of the current-carrying electrodes in addition to the measuring electrodes.

The display-controlling means may be constructed to control the image-displaying means so that the body part undergoing the measurement is visually distinguishable from other body parts. There are various methods available for the visual distinction. For example, the body part undergoing the measurement may be shown in a different color from that of the other parts, or the body part undergoing the measurement may be shown as a blinking image while the other parts is shown as lit images. This construction allows the examiner or the subject to easily check the progress of the measurement by simply looking at the image-displaying means.

In the body composition measurement apparatus according to the second invention, the estimating means may use an estimation formula created based on the result of the measurement of the impedance for the entire body and/or each body part of each of plural pretest subjects, and based on the body composition criteria information of the entire body and/or each body part of each of the pretest subjects measured and collected with an apparatus capable of acquiring cross-sectional images, or further including additional body specific information of the pretest subjects, in order to estimate the information relating to the body composition and/or health condition based on the measurement value of the impedance of each body part of the subject, or based on the measurement value and the body specific information. This construction makes it possible to accurately estimate the information relating to the body composition and health condition, as explained above.

The body composition measurement apparatus according to the second invention may be constructed so that the body specific information includes the height, and the estimating means estimates lengths of limbs or lengths of smaller body parts from the body specific information including at least the height of the subject, obtains body composition information about the limbs or the smaller body parts referring to the estimated value, and visually displays the information. In the case of estimating the body composition information for each body part, the size of the body part is one of the factors that greatly affect the estimation. In general, the size of a body part, such as the length of a limb, is significantly correlated with the height. Taking this into account, the lengths of the limbs or the lengths of the smaller body parts are estimated from the information externally given as body specific information, including the height, and the estimated values of the lengths of the limbs or the lengths of the smaller body parts are used in the estimation of the body composition information from the measurement value of the impedance. This makes it possible to accurately estimate the body composition information.

For a subject with a normal body type, the lengths of the limbs or the lengths of the smaller body parts can be estimated from the height with substantial accuracy. On the other hand, for a person whose body is extraordinarily developed at a particular body part due to trainings, longtime habits or similar activities, such as athletics, the estimation methods prepared for normal individuals with respect to ages, gender and other factors may produce a significant error. To support the measurement for such special subjects, or to further improve the estimation accuracy, it is preferable that the estimated values of the lengths of the limbs or the lengths of the smaller body parts derived from the information including at least the height of the subject can be externally modified.

The body composition measurement apparatus according to the second invention may be constructed so that the body specific information includes the height and the weight, and the apparatus further includes an image-displaying means for displaying information indicating an external body type calculated from the height and the weight, with information indicating an internal body type determined based on the body composition information estimated from the measurement value of the impedance. The “internal body type” principally indicates the state of deposits (or the amounts) of internal fat (or visceral fat). By this construction, the two types of information are displayed together, which will be of greater help to the maintenance and management of the individual's health.

The body composition measurement apparatus according to the second invention may further include an image-displaying means that uses a circular chart to display body composition component ratios based on the body composition information estimated from the measurement values of the impedance, where, for each of plural different body composition types, component ratios are concentrically displayed within each of ranges formed by sectioning the circular chart in radial directions. The “plural different body composition types” are body compositions corresponding to different aspects of the same living body tissues, examples of which are “fat and other components”, “fat, muscle, bone and other components” and “fat, water and other components”. By this construction, the body composition is visually presented in an easy-to-understand form.

The body composition measurement apparatus according to the second invention may preferably include an image-displaying means, which includes a setting display area for entering and setting the body specific information and a result display area for showing the result of the measurement, both located within a single screen. This construction eliminates troublesome operations, such as the switching of screens, during the measurement, so that the time and labor of the measurement is reduced.

In the body composition measurement apparatus according to the second invention, the information relating to the body composition and/or health condition may include the balance of the right and left sides of the body and the balance of the measured segments, or the balance of the upper and lower halves of the body and the balance of the measured segments, with respect to the muscle mass and/or bone mass of four limbs. This construction makes it possible to provide very useful information for athletes, trainees of rehabilitation, or similar individuals.

In the body composition measurement apparatus according to the second invention, the information relating to the body composition and/or health condition may include an ADL index for measuring the ability for activity of daily living. This construction makes it possible to provide very useful information particularly for elderly people, trainees of rehabilitation, or similar individuals.

In the body composition measurement apparatus according to the second invention, the information relating to the body composition and/or health condition may include a bone density of the subject. On the assumption that the bone volume does not change with aging, the water content in the bone increases as the calcium and other highly insulating minerals decrease with aging. This change lowers the electrical characteristic, or impedance, of the bone. Accordingly, the bone density, particularly the decrease in the bone density with aging, can be accurately measured based on the impedance. It is preferable to estimate the bone density based on the impedance of the part in the vicinity of the wrist or ankle, because the ratio of the bone is high at that part. To improve the accuracy of the measurement, the estimation process of the bone density may include a step of correcting the bone density using the impedance of the arm connected to the wrist or the leg connected to the ankle, or using information relating to its size.

In the body composition measurement apparatus according to the second invention, the information relating to the body composition and/or health condition calculated by the body composition measurement apparatus may include basal metabolic rate or energy metabolism of the subject. Among the body component tissues, muscles especially contribute to the basal metabolic rate and the energy metabolism. More especially, the muscles of the lower limbs make greater contribution than those of the upper limbs. Accordingly, the above body composition measurement apparatus may be constructed to estimate the basal metabolic rate or energy metabolism mainly based on the muscle mass of the leg, or of the thigh and the crus, or based on the muscle mass of the entire body including the trunk.

Fat was once regarded as contributing little to the basal metabolic rate or energy metabolism. Lack of consideration of fat, however, may cause a significant error, particularly when the subject is a woman. Therefore, in the above body composition measurement apparatus, it is preferable to estimate the basal metabolic rate or energy metabolism taking account of the fat mass of the entire body or a part of the body.

In a mode of the body composition measurement apparatus according to the second invention, the calculating means and the estimating means are embodied by running a predetermined control program on a multi-purpose personal computer, and the current-generating means and the voltage-measuring means exclusive of the measuring electrodes are enclosed in a main unit contained in a casing with the personal computer. The main unit and the personal computer are capable of communicating with each other. The current-carrying electrodes and the measuring electrodes may be connected to the main unit via cables.

The above construction makes it possible to obtain a body composition measurement apparatus by simply installing a predetermined control program in a personal computer and connecting the personal computer with the main unit. Use of a personal computer, a mass-produced product, reduces the production costs of the apparatus. Users may use their own computers to use the apparatus at still lower costs. The “personal computer” includes any computers regardless of their types, such as a notebook or desktop, and also includes any apparatus having a central processing unit (CPU) and capable of functioning as an information-processing terminal substantially equivalent to a personal computer, in which the control program can be externally installed.

In the above construction, a key operation on the keyboard of the personal computer may be preferably associated with a clicking operation on a button displayed on the screen so that a selecting or commanding operation that requires a user input during the measurement can be similarly performed by either of the key operation and the clicking operation. This allows users to select one of the two input methods according to their preference.

When, as in the case of the body composition measurement apparatus according to the second invention, the body is divided into plural parts to measure the impedance of each body part, it is possible to provide each part with particular characteristics, such as high percentage of osseous tissue or high percentage of muscle tissue. Accordingly, the accuracy of estimation of the body composition of each body part or the entire body can be improved by comparing the measurement results of the impedances of plural body parts. Taking this into account, the body composition measurement apparatus according to the second invention may be constructed to measure at least two of the plural body parts and use the difference or ratio of the body composition information of these body parts estimated from the two measurement values of the impedances of the two body parts or based on the measurement values and the body specific information, to improve the accuracy of estimating the information relating to the body composition of the entire body or a part of the body of the subject.

In this case, it is desirable that the body parts selected have a substantial correlation with each other while each body part has characteristic component ratios of tissues. Therefore, the two body parts are preferably such parts that are serially connected within the body. Examples of the two parts are as follows: upper arm and forearm; forearm and wrist; thigh and crus; and crus and ankle. This is particularly effective in increasing the accuracy of estimating the component ratios of the muscle tissue and osseous tissue, both belonging to the non-fat layer.

Conventionally, the Barthel Index (BI) has been used as a method of evaluating the ADL. The Barthel Index focuses on the ability of self-care and mobility; it allocates scores ranging from 5 to 15 to specified activities such as eating, grooming, dressing, toilet use, bathing, standing/sitting and ambulation. The highest score, 100 points, indicates the perfect independence, while a score of 0 indicates that the person needs assistance in all activities. In addition, recently, the Functional Independence Measure (FIM) has been often used with the BI. These methods, however, cannot avoid providing different evaluations depending on the evaluators, so that the effects of rehabilitation training or the betterment of conditions cannot be promptly reflected. Furthermore, the above methods cannot distinguish the case where the independency is physically impossible, from the case where the independency is caused by psychological or mental factors while the independent life is physically possible, because the scales provided thereby take into account only the kinetic aspects of the subject.

Therefore, it will be very meaningful to propose a quantitative index reflective of the body conditions of the subject, to be used for the ADL evaluation. The body composition measurement apparatus according to the second invention can be constructed to present an ADL index as the information relating to the body composition and/or health condition, as described above. In addition, to solve the above problems, it is possible to propose another body composition measurement apparatus, as a third invention, in which the method of measuring the impedance is not limited.

Accordingly, the body composition measurement apparatus according to the third invention includes:

e) a measuring means for measuring the impedance of almost the entire body or a part of the body of the subject; and

f) an estimating means for estimating an ADL index for measuring the ability for the activities of daily living of the subject based on the measurement value of the impedance, or based on the measurement value and body specific information.

The estimating means may be constructed to estimate the force that the muscle of a predetermined body part essential for the activities of daily living can exhibit, based on the measurement value of the impedance, or based on the measurement value and the body specific information, and to determine the force, or a value derived from the force, as the ADL index.

Examples of the ADL indices are as follows: mass of a muscle necessary for the activities of daily living such as eating, grooming, dressing, toilet use, bathing, standing/sitting and ambulation; (maximum) muscle force that the aforementioned muscle can exhibit; and weight-bearing index, which is a criterion for determining whether a person can continue standing. As described above, based on the measurement value of the impedance of at least one body part, or based on the measurement value and the body specific information, the muscle mass of the above body part or other body parts can be estimated. Accordingly, the estimating means may be constructed to estimate the muscle mass of a predetermined part of the body that is essential for the activities of daily living, based on the measurement value of the impedance, or based on the measurement value and the body specific information, and further estimate, from the muscle mass, the force that the muscle concerned can exhibit. In general, the muscle mass is correlated with the (maximum) muscle force, and the degree of correlation can be known beforehand by experiments. Therefore, the muscle force can be estimated from the estimated muscle mass.

In respect of the ability of the subject to continue standing or to walk, the mass and force of the muscles in the thighs and the crura of the subject are very important. Taking this into account, the body composition measurement apparatus according to the third invention may be constructed so that the muscle of the predetermined part of the body is the muscle included in the thigh or crus of the subject, the measuring means measures the impedance of at least a part of the lower limb of the subject, and the estimating means estimates the mass or muscle force of the muscle included in the thigh or crus based on the measurement value of the impedance, or based on the measurement value and the body specific information. Most preferably, the part to measure the impedance should be identical with the part for which the muscle mass or muscle force must be obtained. However, the muscle mass or muscle force of a desired part can be estimated with substantial accuracy by measuring the impedance of any part of the lower limb, because there is high correlation between the thigh and the crus. One of the most essential muscles that decide whether a person can continue standing or not, is the quadriceps. Taking this into account, in the body composition measurement apparatus, the muscle of the predetermined part of the body may include at least the quadriceps.

If there is an imbalance in muscle mass between the right and left quadricepses, the body will suffer a greater load on one side during a walk or other motion. This will negatively affect future health conditions, such as causing an imbalance in abrasion of bones between the right and left sides. Accordingly, the above body composition measurement apparatus may be preferably constructed to estimate the muscle masses of the right and left quadricepses and provide life-improving advice based on the muscle masses and their right-and-left balance.

The body composition measurement apparatus according to the third invention provides an index for ADL-evaluation of the subject as an objective value derived from the result of measurement of the body of the subject. This eliminates the conventional unevenness in scoring, and makes it possible to provide an objective evaluation on the subject. When, for example, the subject moves from one nursing facility or hospital to another, the value can be commonly used as an index for measuring the ADL, which ensures the continuity of the caring and/or training. Since the ADL is evaluated purely with respect to the physical ability, it is possible to distinguish a subject who is physically able to achieve independency but still in need of nursing care or assistance due to some other reasons. The index immediately reflects the results of medical treatments or rehabilitation training, which greatly helps with the planning of the medical treatments or training and encourages the subject to undergo the treatments or training.

To solve the above problem, the present invention proposes, as a fourth invention, a body composition measurement apparatus, including:

a) plural current-carrying electrodes and plural measuring electrodes to be attached to the body of a subject, to measure the impedance of a target part composed of one body part or two or more body parts connected in series, based on a model in which the entire human body is separated into plural body parts, where the impedance of each body part can be represented by a model composed of impedances respectively corresponding to at least fatty tissue, muscular tissue and osseous tissue connected in parallel, and component ratios of the tissues and electrical characteristics of the whole component tissues and each tissue can be regarded as fixed;

b) a current-supplying means for supplying an alternating current at a preset frequency via the current-carrying electrodes through at least the target part;

c) a voltage-measuring means for measuring the voltage generated by the alternating current between both ends of the target part; and

d) a calculating means for calculating the impedance corresponding to the target part from the voltage measured and the current value of the alternating current, and for estimating information relating to the body composition or health condition of the target part or entire body of the subject from the value of the impedance, or from the value and body specific information, using an estimation formula created based on the result of the measurement of the impedance for the entire body and/or each body part of each of plural pretest subjects, and based on body composition criteria information of the entire body and/or each body part of each of the pretest subjects measured and collected with an apparatus capable of acquiring cross-sectional images, or created by further adding body specific information of the pretest subjects.

As explained above, the body part is a part of the body that can be approximately modeled as a column having a certain length and composed of tissues with their cross-sectional area ratios almost fixed. For example, an arm (from wrist to shoulder or acromion) or leg (from ankle to groin or trochanterion) on either side of the body may be one body part, and a trunk (or idiosoma) may be also one body part. The arm may be further divided at the elbow into forearm and upper arm. Similarly, the leg may be divided at the knee into crus (or lower thigh) and thigh. In the upper limb, the part between the wrist and a point in the vicinity of the roots of the fingers at the back of the hand may be one body part. Similarly, in regard to the lower limb, the part between the ankle and a point in the vicinity of the roots of the fingers at the instep of the foot may be one body part. It is possible to divide any of the aforementioned parts into smaller parts. For example, a part of the forearm close to the wrist may be one body part, and a part of the crus close to the ankle may be one body part.

The body composition measurement apparatus according to the fourth invention supplies a weak alternating current through the current-carrying electrodes into at least one target part, and measures the voltage generated in the current path due to the impedance of the target part, with the voltage-measuring means via the measuring electrodes. Here, well-known four-electrode methods can be used. Even when the attachment positions for the electrodes should be limited, for example when it is preferable to avoid attaching the electrodes to the trunk, the voltage corresponding to the voltage between both ends of the target part can be measured without problem. That is, no potential difference is produced along the voltage measurement path within such parts of the body where the above electric current does not flow, so that such parts can be regarded as mere lead wires as far as the measurement of the voltage is concerned. For example, when the current is supplied between the backs of both hands, the right and left legs and the trunk can be regarded as mere lead wires. There, the voltage measured between the right wrist and the right ankle can be regarded as equal to the voltage drop due to the impedance of the right arm, because the voltage measurement path overlaps the current path only at the right arm. Thus, by appropriately selecting the attachment positions for the current-carrying electrodes and the measuring electrodes, it is possible to obtain the voltage drop across any body part of the subject, and the impedance corresponding to the body part can be derived from the measurement value of the voltage and the current value with the calculating means.

The impedance calculated as described above corresponds to a unitary body part, whose impedance can be approximately represented by a model composed of impedances respectively corresponding to fatty tissue, muscular tissue and osseous tissue connected in parallel, where the component ratios of the tissues and the electrical characteristics of the whole component tissues and each tissue can be regarded as fixed. Each body part resulting from the division closely coincides with the model used as a standard for the calculation of body composition, i.e. with the model used in the MRI method. Therefore, for a body part modeled as described above, the estimation becomes very accurate.

Thus, the body composition measurement apparatus according to the fourth invention makes it possible to accurately estimate not only the composition or other properties of each body part but also information relating to the composition and health condition of the entire body. There is no need to attach electrodes to the trunk even in the case of measuring the impedance of the trunk itself or the impedance of the upper arm, upper limb or other part adjacent to the trunk. This reduces the reluctance that the subject feels, and shortens the binding time for the measurement, because there is no need to remove clothing.

In the body composition measurement apparatus according to the fourth invention, the plural measuring electrodes may be attached to at least two points in the vicinity of the right and left wrists, right and left elbows, right and left knees, palms or backs of the right and left hands, and soles or insteps of the right and left feet. In a mode of the invention, the plural measuring electrodes include at least four electrodes to be attached to four points in the vicinity of the right and left wrists and the right and left ankles. This construction makes it possible to divide the body of the subject into five segments corresponding to the right and left arms, the right and left legs and the trunk, and to obtain the impedance of each segment.

In addition to the aforementioned four points, the attachment points for the measuring electrodes may further include at least one of four points in the vicinity of the right and left elbows and right and left knees. For example, inclusion of all of these four points makes it possible to divide the body of the subject into nine segments corresponding to the right and left upper arms, the right and left forearms, the right and left thighs, the right and left crura and the trunk, and to obtain the impedance of each segment.

The attachment points for the measuring electrodes may further include at least one of four points located at the palms or backs of right and left hands, and the soles or insteps of the right and left feet. For example, inclusion of all of these four points in addition to the aforementioned four points makes it possible to divide the body of the subject into thirteen segments corresponding to the right and left upper arms, the right and left forearms, the right and left wrists, the right and left thighs, the right and left crura, the right and left ankles and the trunk, and to obtain the impedance of each segment.

The attachment points for the measuring electrodes may further include at least one point located between the wrist and the elbow or between the ankle and the knee. This makes it possible to measure the voltage across the wrist-side part of the arm or the ankle-side part of the crus. In these parts, the occupation ratio of the osseous tissue in cross-sectional area is relatively high. Accordingly, these parts are especially suitable for obtaining accurate information about osseous tissue, such as bone mass or bone density.

The attachment points for the current-carrying electrodes may include at least four electrodes to be attached to four points located between the wrists and fingertips of the right and left hands, and between the ankles and fingertips of the right and left feet. When the measuring electrodes are also attached to the wrists and the ankles, the current-carrying electrodes should not be too close to those electrodes. In this case, the current-carrying electrodes may be preferably attached to the backs of the hands or the insteps of the feet at a point in the vicinity of the roots of the fingers, or to the fingers.

To measure the voltage between both ends of the wrist-side part of the arm or the ankle-side part of the crus as described above, it is convenient that two measuring electrodes to be attached to a point in the vicinity of the wrist and a point between the wrist and the elbow are formed on one face of the same sheet-like member, with a predetermined distance between them, and the sheet-like member is affixed to the skin of the subject to perform the measurement. This construction not only facilitates the attachment of the electrodes but also makes it possible to perform the measurement with high accuracy and reproducibility owing to the fixed distance between the two measuring electrodes. Also the current-carrying electrode may be formed on the face of the sheet-like member, which makes the attachment of the electrodes much easier.

In a mode of the body composition measurement apparatus according to the fourth invention, the current-carrying electrodes and the measuring electrodes are attachable to and detachable from the skin, and the electrodes are connected to the current-supplying means and the voltage-measuring means via cables. This construction allows the subject to take any position during the measurement. To improve the accuracy of the measurement, however, it is preferable that the subject takes a supine position to attach the electrodes to the body. More preferably, the subject should rest in the supine position for several minutes before starting the measurement, to balance the body fluid inside the body.

In another mode of the fourth invention, the body composition measurement apparatus further includes a measuring platform for the subject to step on, and grips for the subject to hold with both hands, where the measuring platform has, on its top, the current-carrying electrodes to contact the finger-side parts of the soles and the measuring electrodes to contact the ankle-side part of the soles, and each grip has the measuring electrode to contact a point in the vicinity of the wrist and the current-carrying electrode to contact a predetermined point in the hand. To improve the accuracy of the measurement, the subject should preferably take a standing position, stretching out both arms forwards with both hands holding the grips. This construction allows the subject to perform the measurement in a standing position, and eliminates the necessity to affix the electrodes to the body. Therefore, compared to the case of taking a supine position, the subject feels less reluctant, and the measurement time is shortened. Also, it is easier for the subject to perform the measurement alone.

In another mode of the fourth invention, the body composition measurement apparatus further includes a measuring platform for the subject to step on and a pair of armrests for supporting the arms of the subject standing on the measuring platform with both arms stretched substantially forward, where the measuring platform has, on its top, the current-carrying electrodes to contact the finger-side part of the soles and the measuring electrodes to contact the ankle-side part of the soles, and each armrest has, on its top, the measuring electrode to contact a point in the vicinity of the wrist and the current-carrying electrode to contact a predetermined point in the hand. This construction reduces the fatigue of the subject during the measurement, because the armrests support the arms of the subject. Furthermore, the stable positioning of the arms makes the measurement more accurate, because up-and-down motions of the arms, which causes a measurement error, are prevented during the measurement.

In another mode of the fourth invention, the body composition measurement apparatus further includes a measuring platform for the subject to step on, a chair for the subject to sit down on with the feet placed on the measuring platform, and armrests for the subject to rest the forearms on while sitting on the chair, where the measuring platform has, on its top, the current-carrying electrodes to contact the finger-side part of the soles and the measuring electrodes to contact the ankle-side part of the soles, and each armrest has, on its top, the measuring electrode to contact a point in the vicinity of the wrist and the current-carrying electrode to contact a predetermined point in the hand. This construction allows the subject to take a sitting position during the measurement, so that even those who have difficulties in the standing position can undergo the measurement without difficulty. Furthermore, the construction makes it easier to keep a resting position, so that the reproducibility of measurement is higher than in the case of the standing position.

In concrete, the armrests may have, on their top, a pair of grips to be held with both hands, each grip having the current-carrying electrode. By this construction, when the subject holds the grips, the current-carrying electrode contacts the palm or the finger. The grip may be a substantially columnar body having the current-carrying electrode in the upper part and the measuring electrode in the lower part with a predetermined gap from the current-carrying electrode. By this construction, when the subject holds the grips, the current-carrying electrode touches the finger cushions of the thumb and the index finger, and the measuring electrode touches the ulnar-side part of the palm. The armrest may have, on its top, another measuring electrode to contact a point in the vicinity of the elbow. An ankle-measuring part having a measuring electrode to contact the ankle of the subject may be further provided. A knee-measuring part having a measuring electrode to contact the inside or back of the knee of the subject may be further provided. A measuring electrode to contact the back of the knee may be further provided in the vicinity of the front corner of the seat of the chair. The increase in the number of the voltage-measuring points makes the measurement more accurate.

### BRIEF DESCRIPTION OF THE DRAWINGS

FIG. 1 is an external view of a body composition measurement apparatus as a first embodiment of the present invention.

FIG. 2 is an outlined drawing of the electrical construction of the body composition measurement apparatus of the first embodiment.

FIG. 3 is a detailed drawing of the electrical construction of the body composition measurement apparatus of the first embodiment.

FIG. 4 is a problem analysis diagram showing the initial operation of the measurement operation by the body composition measurement apparatus of the first embodiment.

FIG. 5 is a problem analysis diagram showing the initial operation of the measurement operation by the body composition measurement apparatus of the first embodiment.

FIG. 6 is a flowchart showing the operation in a body composition measurement mode of the body composition measurement apparatus of the first embodiment.

FIG. 7 is a flowchart showing the operation in the body composition measurement mode of the body composition measurement apparatus of the first embodiment.

FIG. 8 is a problem analysis diagram showing the operation of the pre-process in the body composition measurement mode of the body composition measurement apparatus of the first embodiment.

FIG. 9 is a flowchart showing the process of sequentially switching the target part in the body composition measurement mode of the body composition measurement apparatus of the first embodiment.

FIG. 10 is an outlined drawing of the initial screen displayed on the display unit of the body composition measurement apparatus of the first embodiment.

FIG. 11 is an outlined drawing of a screen displayed on the display unit in the body composition measurement mode.

FIG. 12 is a detailed drawing of a part of the screen shown in FIG. 11.

FIG. 13 is a detailed drawing of a part of the screen shown in FIG. 11.

FIG. 14 is a detailed drawing of a part of the screen shown in FIG. 11.

FIG. 15 is a detailed drawing of a part of the screen shown in FIG. 11.

FIGS. 16A and 16B are detailed drawings of a part of the screen shown in FIG. 11.

FIG. 17 is a detailed drawing of a part of the screen shown in FIG. 11.

FIG. 18 is a detailed drawing of a part of the screen shown in FIG. 11.

FIG. 19 is a detailed drawing of a part of the screen shown in FIG. 11.

FIG. 20 is a detailed drawing of a part of the screen shown in FIG. 11.

FIG. 21 is a detailed drawing of a part of the screen shown in FIG. 11.

FIG. 22 is a detailed drawing of a part of the screen shown in FIG. 11.

FIG. 23 is an outlined drawing of a screen displayed on the display unit in the data collection mode.

FIG. 24 is a detailed drawing of a part of the screen shown in FIG. 23.

FIG. 25 is a detailed drawing of a part of the screen shown in FIG. 23.

FIG. 26 is a detailed drawing of a part of the screen shown in FIG. 23.

FIG. 27 is a detailed drawing of a part of the screen shown in FIG. 23.

FIG. 28 is a flowchart showing the measurement operation in the body composition measurement mode of the body composition measurement apparatus of the first embodiment.

FIG. 29 is a flowchart showing the measurement operation in another body composition measurement mode of the body composition measurement apparatus of the first embodiment.

FIG. 30 is a figurative drawing showing the attachment positions for the electrodes in the body composition measurement mode of the body composition measurement apparatus of the first embodiment.

FIG. 31 is a perspective view showing a recommended position for the measurement of the body composition using the body composition measurement apparatus of the first embodiment.

FIG. 32 shows an impedance model of the human body corresponding to the body composition measurement method according to the present invention.

FIG. 33A shows a model illustrating the state of acquisition of cross-sectional images with an MRI in the body composition measurement method according to the present invention, and FIG. 33B is an example of a distribution chart of the mass of a tissue for each section of the body part.

FIG. 34A shows a composition model of a segment resulting from the division of the body in the body composition measurement method according to the present invention, and FIG. 34B shows an equivalent circuit model of the tissues.

FIG. 35 is an outlined drawing of the electrical construction of a body composition measurement apparatus, as a modified form of the first embodiment.

FIG. 36 is an external view showing a modified structure of an electrode of the body composition measurement apparatus of the first embodiment.

FIG. 37 shows an impedance model of the human body corresponding to another body composition measurement method according to the present invention.

FIG. 38 shows an electrode pad of a body composition measurement apparatus as the second embodiment of the present invention, attached to the body.

FIG. 39 is an external view of the electrode pad used in the body composition measurement apparatus of the second embodiment.

FIG. 40 shows a modified form of the electrode pad in the second embodiment, which is attached to the body.

FIG. 41 shows a modified form of the electrode pad in the second embodiment, which is attached to the body.

FIG. 42 shows the state of use of a body composition measurement apparatus as the third embodiment of the present invention.

FIG. 43 is an external perspective view of the lower limb measuring unit of the body composition measurement apparatus of the third embodiment.

FIG. 44 is an enlarged view showing the state of measurement using the lower limb measuring unit shown in FIG. 43.

FIG. 45 is an external perspective view of the upper limb measuring unit of the body composition measurement apparatus of the third embodiment.

FIG. 46 shows the electrical construction of the body composition measurement apparatus of the third embodiment.

FIG. 47 is a flowchart showing the measurement operation of the body composition measurement apparatus of the third embodiment.

FIG. 48 is an external perspective view showing a modified form of the lower limb measuring unit of the body composition measurement apparatus of the third embodiment.

FIG. 49 is an external view of a body composition measurement apparatus as the fourth embodiment of the present invention.

FIG. 50 is an external view of a body composition measurement apparatus as the fifth embodiment of the present invention.

FIG. 51 is an enlarged view of a grip of the body composition measurement apparatus in the fourth or fifth embodiments.

FIG. 52 shows the state of use of the body composition measurement apparatus of the fourth embodiment.

FIG. 53 is a front view of the measuring platform (and its surroundings) of the body composition measurement apparatus of the fifth embodiment.

### BEST MODE FOR CARRYING OUT THE INVENTION

The body composition measurement method and apparatus according to the present invention is described in detail, referring to the drawings. First, the description focuses on the method of measuring the impedance relating to the body composition measurement method according to the present invention and the method of estimating body composition information based on the measurement value of the impedance or based on the measurement value and body specific information.

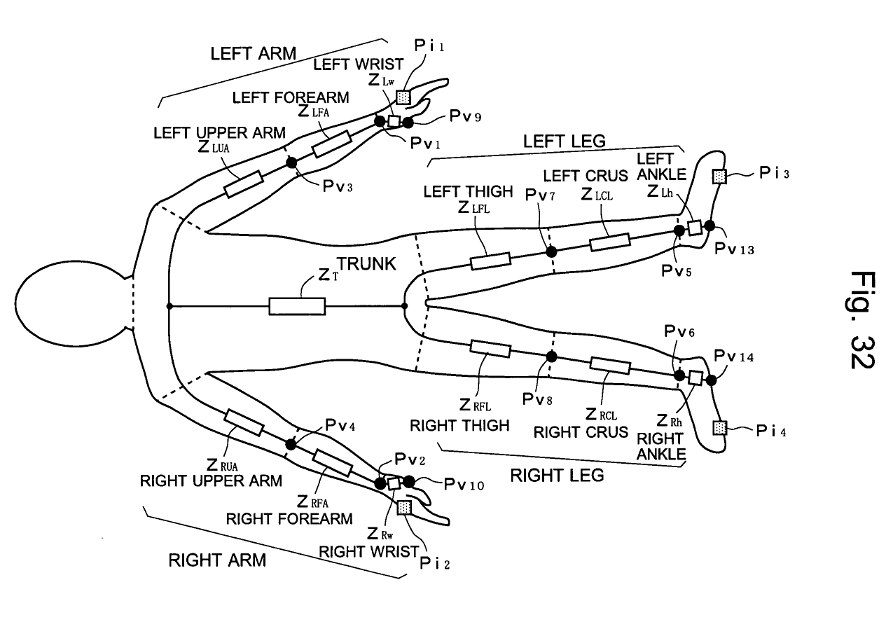

FIG. 32 shows an approximate model representing the impedance configuration of the human body corresponding to the present body composition measurement method. One feature of the present method is that the human body is divided into plural segments, and the impedance is considered for each segment. To improve the accuracy of estimation of the body composition information based on the impedance, the segment is defined corresponding to every part of the body where the composition of the tissue is relatively uniform, i.e. every part of the body that can be approximately represented by a columnar model, which will be explained later.

In detail, as shown in FIG. 32, the right and left arms (exclusive of the hands) are each divided at the elbow into the upper arm and the forearm, and the right and left legs (exclusive of the feet) are each divided at the knee into the thigh and the crus. Thus, the four limbs are divided into eight segments. In addition, the trunk (including the chest and the abdomen) is considered as one segment. The head and the fingertips of the hands and the feet are excluded from consideration for the present. Thus, the entire body is divided into nine segments. Each of the nine segments is independently assigned an impedance, and a model is created in which the impedances are connected as shown in FIG. 32. In FIG. 32, Z_{LFA}, Z_{LUA}, Z_{RFA}, Z_{RUA}, Z_{LFL}, Z_{LCL}, Z_{RFL}, Z_{RCL}, and Z_{T} denote the impedances of the left forearm, left upper arm, right forearm, right upper arm, left thigh, left crus, right thigh, right crus and trunk, respectively.

To measure the impedances of the nine segments, four current-supplying points (Pi₁ to Pi₄) and eight voltage-measuring points (Pv₁ to Pv₈) are determined on the limbs of the subject in a supine position, as shown in FIG. 32. The current-supplying points Pi₁ to Pi₄ are located in the vicinity of the root of the middle fingers on the backs of both hands and in the vicinity of the roots of the middle fingers on the insteps of both feet. The voltage-measuring points Pv₁ to Pv₈ are located at the right and left wrists, right and left elbows, right and left ankles and right and left knees. The voltage-measuring points Pv₁ and Pv₂ at the right and left wrists and Pv₅ and Pv₆ at the right and left ankles are relatively distant from the trunk. Therefore, the measurement of voltage using these four voltage-measuring points is called the “distal measurement” hereinafter. On the other hand, the voltage-measuring points Pv₃ and Pv₄ at the right and left elbows and Pv₇ and Pv₈ at the right and left knees are relatively close to the trunk. Therefore, the measurement of voltage using these four voltage-measuring points is called the “proximal measurement” hereinafter. As shown in FIG. 32, the parts located on the hands and feet (or distal sides) can be regarded as having their own impedances and these impedances are denoted by Z_{Lw}, Z_{Rw}, Z_{Lh} and Z_{Rh}.

When electric current is supplied between the two points selected from the four current-supplying points Pi₁ to Pi₄, the potential difference between a predetermined pair of the voltage-measuring points can be regarded as a potential difference generated between both ends of an impedance, or between both ends of plural impedances connected in series. In this case, the current barely flows through such parts of the body that are not on the current path. Therefore, it is possible to regard such parts as mere lead wires, ignoring their impedances.

Suppose the current is supplied between the current-supplying points Pi₁ and Pi₂ located at both hands. In this case, the potential difference between the voltage-measuring points Pv₁ and Pv₂ located at both wrists (i.e. distal measurement) is equal to the voltage corresponding to the impedance composed of Z_{LFA}, Z_{LUA}, Z_{RFA} and Z_{RUA} connected in series; i.e. the impedance of the right and left arms. The potential difference between the voltage-measuring points Pv₃ and Pv₄ located at both elbows (i.e. proximal measurement) is equal to the voltage corresponding to the impedance Z_{LUA} and Z_{RUA} connected in series; i.e. the impedance of the right and left upper arms. The potential difference between the voltage-measuring points Pv₁ located at the left wrist and Pv₅ located at the left ankle (or Pv₆ located at the right ankle) is equal to the voltage corresponding to the impedance Z_{LFA} and Z_{LUA} connected in series, i.e. the impedance of the left arm, because the right and left legs and the trunk can be regarded as mere lead wires. The potential difference between the voltage-measuring points Pv₃ located at the left elbow and Pv₇ located at the left knee (or Pv₈ located at the right knee) is equal to the voltage corresponding to the impedance Z_{LUA}, i.e. the impedance of the left upper arm, because the right and left thighs and the trunk can be regarded as mere lead wires.

The measurement can be similarly performed for other parts of the body. These measurement results make it possible to accurately measure the impedance of each of the nine segments. Based on the measurement values of the impedance, or based on the measurement value of the impedance and body specific information, the body composition information is estimated.

As will be described later, the present body composition measurement apparatus uses four measuring electrodes, and allows the user to select one of the following methods of measuring the impedance: distal measurement only; proximal measurement only; both distal and proximal measurements by rearranging the measuring electrodes.

Methods of estimating body composition information based on the measurement value of the impedance obtained as described above are explained below. One remarkable feature of the methods used in the present body composition measurement apparatus is that they use estimation formulae, developed on the basis of the body composition information collected with an MRI, to estimate body composition information based on the measurement value of the impedance, or based on the measurement value of the impedance and the body specific information.

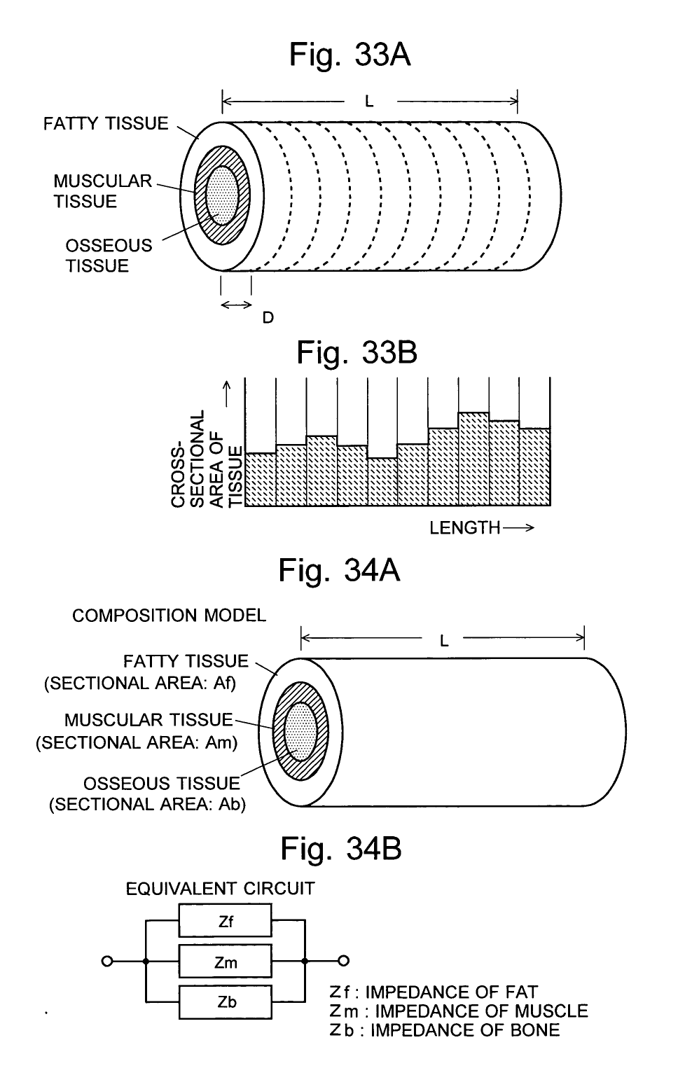

An MRI is capable of acquiring cross-sectional images of any part of the human body. The cross-sectional images provide information about the masses and/or ratios of the tissues, such as muscle, fat and bone, within the cross section. FIG. 33A shows cross-sectional images acquired for each thickness D along the longitudinal direction of a body part. From these images, the masses (or areas) of the tissues, such as muscle, fat and bone, are calculated. From this calculation, the area distribution of the tissues along the longitudinal direction of the body part can be obtained, as shown in FIG. 33B. Then, the areas are integrated along the longitudinal direction to determine the masses of the tissues within the body part concerned. In the present measurement method, the body is divided into nine segments, so that it is easy to perform the MRI measurement for each unitary segment. Furthermore, since each segment is defined so that it approximates a columnar body, the mass of each tissue can be obtained with high accuracy.

The following description provides important examples of the methods of estimating principal body composition information to be displayed by the present body composition measurement apparatus as the result of the measurement.

[1] Estimation of Composition of the Entire Body

The “composition” includes: body-fat ratio (% Fat), lean body mass (LBM), fat mass (FM), etc.

[1-1] Example of Estimation of Total Body-Fat Ratio

Based on the study of Lukaski et. al., the following formula has been conventionally used to estimate lean body mass by bioelectrical impedance methods:

LBM [kg]=a₀+b₀×(H²/Z₁)+c₀×W+d₀×Ag,

where a₀, b₀, c₀ and d₀ are constants (multiple regression coefficients) whose values differ according to the gender (Sx), and H, W, Ag and Z₁ are the height, weight, age and impedance between wrist and ankle, respectively.

Using the lean body mass (LBM) and the weight (W), the body-fat ratio (% Fat) is given by:

% Fat=[(W−LBM)]/W]×100,

and the fat mass (FM) is given by:

FM=W−LBM.

In the present measurement method, however, the lean body mass (LBM) is not calculated by the above formula, but by another method, which will be described later.

[1-2] Example of Estimation of Total Lean Body Mass

With the nine segments of the body each regarded as a columnar model, the body composition is estimated by either of the following two methods.

[1-2-1] First method: Consider four limbs and trunk as independent variables, and create multiple regression formula.

First, the entire body is divided into five segments: i.e. four limbs and trunk. Denoting the total lean body mass as LBM, lean body mass of the right and left arms as LBMₕ, lean body mass of the right and left legs as LBM_{L}, and lean body mass of the trunk as LBMₜᵣ, the following formulae are obtained:

LBMₕ∝Hₕ²/Zₕ

where Hₕ is the length of one or both arms and Zₕ is the impedance of one or both arms,

LBM_{L}∝H_{L}²/Z_{L}

where H_{L} is the length of one or both legs and Z_{L} is the impedance of one or both legs,

LBMₜᵣ∝Hₜᵣ²/Zₜᵣ

where Hₜᵣ is the length of the trunk and Zₜᵣ is the impedance of the trunk.

From these formulae, the following formula is obtained:

LBM=a₀+b₀×Hₕ²/Zₕ+c₀×H_{L}²/Z_{L}+d₀×Hₜᵣ²/Zₜᵣ+e₀×W+f₀×Ag (1),

where weight (W) and age (Ag) are supplementary variables for improving the correlation. The term “Ag” corrects the difference in properties of the tissues depending on the age, and the term “W” compensates for the influences on the bone density or other characteristics caused by the weight stressing the osseous tissue. Naturally, the values of the coefficients a₀, b₀, c₀, d₀, e₀ and f₀ differ depending on the gender (Sx).

In general, Hₕ, H_{L} and Hₜᵣ are highly correlated with the height (H) of the subject. Accordingly, formula (1) can be rewritten as follows by substituting H for Hₕ, H_{L} and Hₜᵣ:

LBM=a₀′+b₀′×H²/Zₕ+c₀′×H²/Z_{L}+d₀′×H²/Zₜᵣ+e₀′×W+f₀′×Ag (2).

Here, Zₕ may be either the impedance of both arms or the impedance of one arm. In the case of one arm, it is assumed that both arms have the same impedance. The same argument also holds true for Z_{L}.

When four limbs are regarded as independent, then formula (1) will be rewritten as follows:

LBM=a₀″+b₀″×H_{hR}²/Z_{hR}+c₀″×H_{hL}²/Z_{hL}+d₀″×H_{LR}²/Z_{LR}+e₀″×H_{LL}²/Z_{LL}+f₀″×Hₜᵣ²/Zₜᵣ+g₀″×W+h₀″×Ag (3),

where

H_{hR} is the length of the right arm, and Z_{hR} is the impedance of the right arm,

H_{hL} is the length of the left arm, and Z_{hL} is the impedance of the left arm,

H_{LR} is the length of the right leg, and Z_{LR} is the impedance of the right leg, and

H_{LL} is the length of the left leg, and Z_{LL} is the impedance of the left leg.

Furthermore, when the measurement can be performed for each of the nine segments, formula (1) will be rewritten as follows:

$$LBM = {a}_{0} + {b}_{0} \times {H}_{UAR}^{2} / {Z}_{UAR} + {c}_{0} \times {H}_{FAR}^{2} / {Z}_{FAR} + {d}_{0} \times {H}_{UAL}^{2} / {Z}_{UAL} + {e}_{0} \times {H}_{FAL}^{2} / {Z}_{FAL} + {f}_{0} \times {H}_{FLR}^{2} / {Z}_{FLR} + {g}_{0} \times {H}_{CLR}^{2} / {Z}_{CLR} + {h}_{0} {H}_{FLL}^{2} / {Z}_{FLL} + {i}_{0} \times {H}_{CLL}^{2} / {Z}_{CLL} + {j}_{0} \times {H}_{tr}^{2} / {Z}_{tr} + {k}_{0} \times W + {l}_{0} \times Ag . \qquad ( 4 )$$

It should be noted that any of the above formula (1), (2), (3) and (4) does not necessarily include all the variables as shown above; the formula may be preferably composed of only independent variables that are essentially effective. The above formulae should be regarded as examples where the maximal number of variables are used.

[1-2-2] Second method: Estimate body composition for each segment, and include the estimated values into the formula for estimating the total body composition.

Denoting the lean body mass of an arm as LBMₕ, the lean body mass of a leg as LBM_{L}, and the lean body mass of the trunk as LBMₜᵣ, the following formulae are obtained:

$$LBM = {a}_{0} + {b}_{0} \times {LBM}_{h} + {c}_{0} \times {LBM}_{L} + {d}_{0} \times {LBM}_{tr} , \\ {LBM}_{h} = {a}_{1} + {b}_{1} \times {H}_{h}^{2} / {Z}_{h} + {c}_{1} \times W + {d}_{1} \times Ag , \\ {LBM}_{L} = {a}_{2} + {b}_{2} \times {H}_{L}^{2} / {Z}_{L} + {c}_{2} \times W + {d}_{2} \times Ag , \\ {LBM}_{tr} = {a}_{3} + {b}_{3} \times {H}_{tr}^{2} / {Z}_{tr} + {c}_{3} \times W + {d}_{3} \times Ag . \qquad ( 5 )$$

Formula (5) corresponds to formula (1). It is also possible to create formulae corresponding to formulae (3) and (4).

[1-3] Estimation of Total Muscle Mass and Total Bone Mass

Conventional anatomic data or similar data generally suggests that total muscle mass (TMM) is about 50% of lean body mass (LBM), and total bone mass (TBM) is about 16% of weight (W) or about 18% of lean body mass (LBM). Using these values, it is easy to roughly estimate the total muscle mass (TMM) and the total bone mass (TBM) from the lean body mass (LBM) and weight (W) obtained as described above. Furthermore, total muscle mass (TMM) and total bone mass (TBM) have significant correlation with lean body mass (LBM). Therefore, it is also possible to create multiple regression formulae using variables similar to those used in the estimation formulae of LBM.

TMM=a₀+b₀×H²/Z₁+c₀×W+d₀×Ag

TBM=a₁+b₁×H²/Z₁+c₁×W+d₁×Ag

The above formulae are the simplest ones. As explained above, it is possible to create more complex formulae for more accurate estimation.

[2] Estimation of Body Composition of Each Segment Unit

[2-1] Estimation of Lean Body Mass

Columnar composition model is applied to each of the nine segments. FIG. 34A shows the composition model of each segment. Each segment is assumed to contain the fatty tissue having cross-sectional area A_{f}, muscular tissue having cross-sectional area Aₘ and osseous tissue having cross-sectional area A_{b}, all tissues having the same length L. Denoting the volume resistivity of the fatty tissue, muscular tissue and osseous tissue as ρ_{f}, ρₘ and ρ_{b}, respectively, the impedance Z_{f}, Zₘ and Z_{b} of the fatty tissue, muscular tissue and osseous tissue are given as follows:

Z_{f}=ρ_{f}×(L/A_{f}),

Zₘ=ρₘ×(L/Aₘ),

Z_{b}=ρ_{b}×(L/A_{b}).

The impedance Z₀ of the entire segment can be approximately represented by a model composed of the impedances Z_{f}, Zₘ and Z_{b} connected in parallel, as shown in FIG. 34B.

Accordingly, the impedance Z₀ is given as follows:

1/Z₀=(1/Z_{f})+(1/Zₘ)+(1/Z_{b}) (11).

Now, the volume and density of non-fat layer (the layer other than the fat layer or panniculus) are denoted by V_{LBM} and D_{LBM}. The density D_{LBM} is known from conventional studies. Then, the lean body mass (LBM) is given by:

LBM=V_{LBM}×D_{LBM,}

where

$$\begin{matrix}{V}_{LBM} = {A}_{LBM} \times L \\ = ( {A}_{m} + {A}_{b} ) \times L \\ = {ρ}_{m} \times ( {L}^{2} / {Z}_{m} ) + {ρ}_{b} \times ( {L}^{2} / {Z}_{b} ) .\end{matrix} \qquad ( 12 )$$

Substituting formula (11) into formula (12),

V_{LBM}=ρₘ×L₂×[1/Z₀−1/Z_{f}]+(ρ_{b}−ρₘ)×(L²/Z_{b}) (13),

where the volume resistivities of the tissues satisfy the condition: ρₘ<ρ_{b}<<ρ_{f}.

It is assumed here that there is no influence from distal parts such as wrists or ankles (Condition A). Then

A_{b}<<Aₘ.

Therefore,

Z_{f}(=ρ_{f}×L/A_{f})>Z_{b}(=ρ_{b}×L/A_{b})>>Zₘ(=ρₘ×L/Aₘ)>Z₀.

Applying this to formula (13),

V_{LBM}=ρₘ×(L²/Z₀)+(ρ_{b}−ρₘ)×(L²/Z_{b}) (14).

Here,

ρₘ×(L²/Z₀)>>(ρ_{b}−ρₘ)×(L²/Z_{b}),

so that

V_{LBM}=ρₘ×(L²/Z₀),

and

LBM=D_{LBM}×ρₘ×(L²/Z₀).

Therefore, using a predetermined function f(x), the following relation is obtained:

LBM=f(L²/Z₀).

Now, the influences from distal parts, such as wrists or ankles, are taken into account (Condition B). In this case,

A_{b}<Aₘ.

Therefore

ρₘ×(L²/Z₀)>(ρ_{b}−ρₘ)×(L²/Z_{b})=ΔV_{b}.

In general, the heavier the body is, the greater the volume V_{b} of the osseous tissue becomes to support the body. Accordingly, the following relation can be assumed: V_{b}∝ΔV_{b}∝f(W).

Therefore, from formula (14),

$$\begin{matrix}{V}_{LBM} = {ρ}_{m} \times ( {L}^{2} / {Z}_{0} ) + ( {ρ}_{b} - {ρ}_{m} ) \times ( {L}^{2} / {Z}_{b} ) \\ = {ρ}_{m} \times ( {L}^{2} / {Z}_{0} ) + Δ {V}_{b} \\ \approx {ρ}_{m} \times ( {L}^{2} / {Z}_{0} ) + f ( W ) ,\end{matrix}$$

so that

LBM=f(L²/Z₀, W).

Taking into account the change of tissues with aging and the difference depending on genders, the estimation formula for multiple regression analysis can be created as follows:

LBM=a″+b″×(L²/Z₀)+c″×W+d″×Ag (15),

where a″, b″, c″ and d″ are constants (multiple regression coefficients), which take different values for different genders. The values of a″, b″, c″ and d″ for each gender can be determined beforehand by applying the lean body mass (LBM) obtained by an MRI method to the estimation formula for multiple regression analysis.

[2-2] Estimation of Muscle Mass

This is basically the same as the above-described estimation of lean body mass. Denoting the volume and density of the muscle layer as V_{MM} and D_{MM}, respectively, the muscle mass MM is given by:

MM=V_{MM}×D_{MM}.

Using the impedance Zₘ of the muscle layer,

V_{MM}=ρₘ×(L²/Zₘ).

Under the condition A,

MM≈LBM=a+b×(L²/Z₀)+c×Ag (16).

Under the condition B, on the other hand,

$$\begin{matrix}LBM = MM + BM \\ = a + b \times ( {L}^{2} / {Z}_{0} ) + c \times W + d \times Ag .\end{matrix} \qquad ( 17 )$$

In formula (17), the term L²/Z₀ contains information about bone (BM) in addition to the muscle mass (MM); it is impossible to separate them. Among the nine segments, upper arms and thighs satisfy the condition A, and forearms and crura satisfy the condition B.

It is known that, for each person, the muscle mass of the upper arm is highly correlated with that of the forearm, and the muscle mass of the thigh is highly correlated with that of the crus. Accordingly, information about muscle mass of the upper arm (MM_{U}) and information about muscle mass of the forearm (MM_{F}) are obtained as follows. That is, based on the regression analysis of MM_{UA} and MM_{FA} obtained by an MRI method, the following estimation formula is created:

MM_{FA}=aₘ+bₘ×MM_{UA} (18).

Similarly, muscle mass of the crus (MM_{CL}) is estimated from the information about the muscle mass of the thigh (MM_{FL}) obtained by an MRI method:

MM_{CL}=a′ₘ+b′ₘ×MM_{FL} (19).

The muscle mass of a proximal segment, such as the upper arm or thigh, can be obtained by formula (16) because it satisfies the condition A. Further, by substituting the muscle masses of the upper arm and the thigh, obtained by formula (16), into formula (18) and (19), the muscle masses of the forearm that of the crus can be estimated.

[2-3] Estimation of Bone Mass

Paying attention to the forearm and the crus, both satisfying the condition B, the bone masses BM_{FA} and BM_{CL} can be calculated by subtracting MM_{FA} and MM_{CL}, calculated by formula (18) and (19), from the lean body masses LBM_{FA} and LBM_{CL}, calculated by formula (15), respectively:

BM_{FA}=LBM_{FA}−MM_{FA} (20),

BM_{CL}=LBM_{CL}−MM_{CL} (20).

Based on the bone masses obtained by formulae (20) and (21), the bone masses of other segments, each satisfying the condition A, and the total bone mass are estimated. Similar to the case of muscle mass, bone mass of the upper arm is highly correlated with that of the forearm, and bone mass of the thigh is highly correlated with that of the crus for each subject. Accordingly, based on the regression analysis of BM_{FA} and BM_{CL} obtained by an MRI method, the following estimation formulae can be created:

BM_{UA}=a_{b}+b_{b}×BM_{FA} (22),

BM_{FL}=a′_{b}+b′_{b}×BM_{CL} (23).

Similarly, it is possible to create estimation formulae based on the total bone mass and the regression analysis of the arms and legs by an MRI method. The above estimation method has assumed that the lean body mass, muscle mass, muscle force, bone mass, etc., are estimated for each segment. In some cases, the result becomes more accurate when the estimation formulae are created so that the lean body mass, muscle mass, muscle force, bone mass, etc., should be estimated for each unit length within the segment. This method is particularly effective in the case where the subject is an athlete or other type of person who has such a special body type that the segment lengths or other sizes of the upper arm and forearm, or those of the thigh and crus, are greatly unbalanced between the right and left sides of the body.

An example of estimation of muscle mass and bone mass per unit length is described below. The volume (V), cross-sectional area (A) and length (L) of a columnar model satisfy the following relation:

V=A×L.

Therefore,

V/L=A=ρ×(L/Z).

Formulae (16)-(23) can be rewritten into per-unit-length forms as follows:

$$\begin{matrix}\begin{matrix}MM / L \approx LBM / L \\ = a + b \times ( L / {Z}_{0} ) + c \times Ag ,\end{matrix} & {( 16 )}^{′} \\ \begin{matrix}LBM / L = ( MM + BM ) / L \\ = a + b \times ( L / {Z}_{0} ) + c \times W + d \times Ag ,\end{matrix} & {( 17 )}^{′}\end{matrix}$$

MM_{FA}/L_{FA}=aₘ+bₘ×MM_{UA}/L_{UA} (18)′,

MM_{CL}/L_{CL}=a′ₘ+b′ₘ×MM_{FL}/L_{FL} (19)′,

BM_{FA}/L_{FA}=LBM_{FA}/L_{FA}−MM_{FA}/L_{FA} (20)′,

BM_{CL}/L_{CL}=LBM_{CL}/L_{CL}−MM_{CL}/L_{CL} (21)′,

BM_{UA}/L_{UA}=a_{b}+b_{b}×BM_{FA}/L_{UA} (22)′,

BM_{FL}/L_{FL}=a′_{b}+b′_{b}×BM_{CL}/L_{CL} (23)′,

Therefore,

MM_{UA}=(MM_{UA}/L_{UA})×L_{UA},

MM_{FA}=(MM_{FA}/L_{FA})×L_{FA},

MM_{FL}=(MM_{FL}/L_{FL})×L_{FL},

MM_{CL}=(MM_{CL}/L_{CL})×L_{CL},

LBM_{FA}=(LBM_{FA}/L_{FA})×L_{FA},

LBM_{CL}=(LBM_{CL}/L_{CL})×L_{CL},

BM_{UA}=(BM_{UA}/L_{UA})×L_{UA},

BM_{FA}=(BM_{FA}/L_{FA})×L_{FA},

BM_{FL}=(BM_{FL}/L_{FL})×L_{FL},

BM_{CL}=(BM_{CL}/L_{CL})×L_{CL}.

Expressions using the function f(x) are as follows:

MM_{UA}=f(L_{UA}²/Z_{UA}), or f(L_{UA}²/Z_{UA}, W, Ag),

MM_{FL}=f(L_{FL}²/Z_{FL}), or f(L_{FL}²/Z_{FL}, W, Ag),

MM_{FA}=f(L_{FA}²/Z_{FA}, L_{UA}²/Z_{UA}, W, Ag), or f(L_{FA}²/Z_{FA}, L_{UA}²/Z_{UA}, W, Ag)×L_{FA},

MM_{CL}=f(L_{CL}²/Z_{CL}, L_{FL}²/Z_{FL}, W, Ag), or f(L_{CL}²/Z_{CL}, L_{FL}²/Z_{FL}, W, Ag)×L_{CL}.

[3] Estimation of Basal Metabolic Rate

A general method of estimating basal metabolic rate (BM) is as follows:

$$\begin{matrix}BM \text{[kCal]/day} \approx RM / 1.2 \\ ∝ {VO}_{2} r \text{[mL/min]} \\ ∝ LBM \text{[kg]} \\ ∝ TMM \text{[kg]} ,\end{matrix}$$

where RM is resting metabolic rate, VO₂r is resting oxygen intake, LBM is lean body mass and TMM is total muscle mass. For example, when LBM=59.9 kg, then

$$\begin{matrix}{VO}_{2} r = ( LBM + 7.36 ) / 0.2929 \\ = 229.635 \text{[mL/min]} .\end{matrix}$$

If respiratory quotient (RQ) is 0.82 (constant), one liter of oxygen gas produces 4.825 kCal of heat. Accordingly, the daily oxygen consumption is:

229.635[mL/min]×60[min]×24[hr]=330.674[L],

and the basal metabolic rate (BM) is:

BM=4.825[kCal]×330.674=1595.5[kCal].

The following discussion focuses on muscles as constituent tissues of the lean body mass (LBM). The present measurement method can accurately estimate the muscle mass of each segment. Therefore, it is expected that basal metabolic rate (BM) and resting metabolic rate (RM) can be estimated more accurately by using total muscle mass (TMM) rather than lean body mass (LBM). Taking this into account, multiple regression formulae may be created as follows:

BM(or RM)=f(TMM),

or

BM(or RM)=f(MM of each segment).

Furthermore, it is expected that muscles of different parts of the body differently contributes to the basal metabolism. For example, the muscles of the legs will contribute to the basal metabolism more than the muscles of the arms. This suggests that the muscle mass of the leg (thigh and crus) is more correlated with the basal metabolic rate (BM) and the resting metabolic rate (RM) than the total muscle mass (TMM). Taking this into account, multiple regression formulae may be created as follows:

BM(or RM)=f(MM_{FL}, MM_{CL}).

Conventionally, fatty tissue has been excluded from consideration because it contributes little to the basal metabolism. It is true that fat tissue is less active than muscular tissue, but it can still contribute to the metabolism to some extent. Therefore, in order to improve the estimation accuracy, it is desirable to create an estimation formula that takes into account fatty tissues. Accordingly, using fat mass (FM), a multiple regression formula may be created as follows:

BM(or RM)=f(TMM, FM).

It is said that the basal metabolic rate, particularly that of women, is not always highly correlated with the lean body mass; it is rather correlated with weight. This suggests that the metabolism of fat tissue is not ignorable. The present measurement method can accurately estimate fat mass (FM). By using this fat mass, it is possible to effectively improve the accuracy of estimation of basal metabolic rate.

[4] Estimation of ADL Index

ADL index proposed here is an index for determining to what degree an elderly person, a person subjected to medical treatments because of illness or injury, or other individual has the ability for to live a physically independent everyday life. This index is intended to replace or supplement the Barthel Index and/or FIM conventionally used as ADL evaluation methods. In ADL evaluation, it is necessary to evaluate motions corresponding to various activities of daily living of the human being. The ADL index to be given by the present apparatus mainly focuses on the ability of the subject to continue standing. In this embodiment, muscle mass of the quadriceps, maximum muscle force of the quadriceps and weight-bearing index are used as the ADL indices, but other indices may be used. Muscle mass of the quadriceps is highly correlated with muscle mass of the leg or thigh, which includes the quadriceps as its component. Therefore, the muscle mass of the quadriceps can be easily estimated from that of the leg or thigh calculated as described above. The maximum muscle force of the quadriceps can be easily estimated from the muscle mass of the quadriceps, because maximum muscle force is highly correlated with muscle mass. Furthermore, the weight-bearing index can be estimated from the maximum muscle force of the quadriceps and the body weight

As described above, the present measurement method makes it possible to estimate information that reflects the body composition and/or health condition, such as masses of tissues or basal metabolic rate, from measurement values of the impedance, based on the regression analysis of masses of tissues obtained by an MRI method.

[First Embodiment]

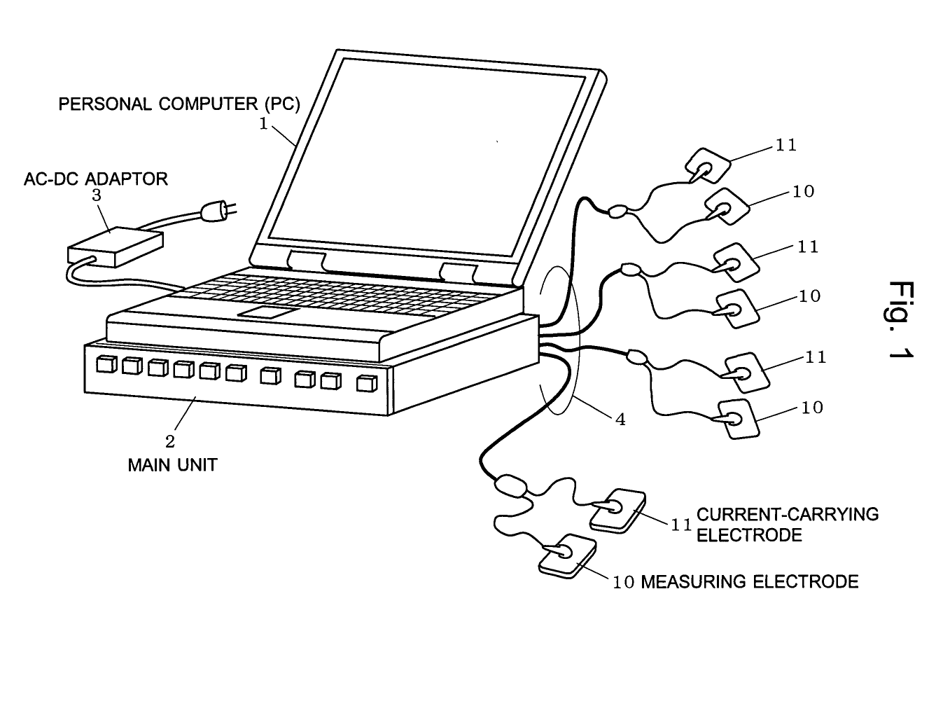

Next, the construction and operation of a body composition measurement apparatus as the first embodiment of the present invention is described. FIG. 1 is an external view of body composition measurement apparatus of the present embodiment.

The present body composition measurement apparatus is constructed to supply a weak radio frequency current into the body of the subject, to detect the voltage generated by the current in a predetermined part of the body, to calculate the impedance from the voltage value and the current value, to perform a calculation wherein the measurement value of the impedance and the body specific information, such as the height, weight, age and gender, which have been externally entered, are applied to a predetermined estimation formula, and to calculate and show the body composition information (such as body-fat ratio, lean body mass, body-fat mass, total body water, muscle mass, muscle force, bone mass, bone density, degree of obesity, basal metabolic rate and ADL index) and health condition information of the subject. The present apparatus estimates various kinds of body composition information, as described above. Particularly, the apparatus is constructed to display a variety of information relating to the result of the measurement of muscle masses.

As shown in FIG. 1, the present body composition measurement apparatus includes a notebook-size personal computer (which is referred to as the “PC” hereinafter) 1 used mainly as a controller and data processor, and a main unit 2 used mainly for measuring impedances. Plural electrodes necessary for the measurement are taken out from the back of the main unit 2 via cables 4. The power cable for taking commercial power supply is connected to the main unit 2 via an AC-DC adapter 3. The electrodes include current-supplying electrodes 10 for supplying the current and measuring electrodes 11 for measuring the voltage. One current-carrying electrode 10 is paired with one measuring electrode 11, and the pair is connected via a low-inductive cable 4 to the main unit 2. The current-carrying electrodes 10 and the measuring electrodes 11 can be securely and immovably attached to the skin of the subject. Each electrode is designed to have a flat affix-type body so as to decrease the impedance, or contact resistance, of the electrode itself.

The present body composition measurement apparatus is designed to measure the impedance by measuring the voltage for up to sixteen voltage-measuring points, as will be described later, using four current-carrying electrodes 10 and four measuring electrodes 11, paired with each other. When, as will be described later, the measurement should be performed for eight or sixteen voltage-measuring points, the examiner should rearrange the measuring electrodes 11 on the body of the subject after every measurement for every four points. This is because use of a large number of electrodes would not only increase the production costs of the apparatus, but also makes the preparation for the measurement more troublesome due to the entanglement of the cables. Furthermore, mistaken attachments of electrodes to the subject would be more probable. In the case where these problems do not matter, the apparatus may be designed to have eight or sixteen measuring electrodes.

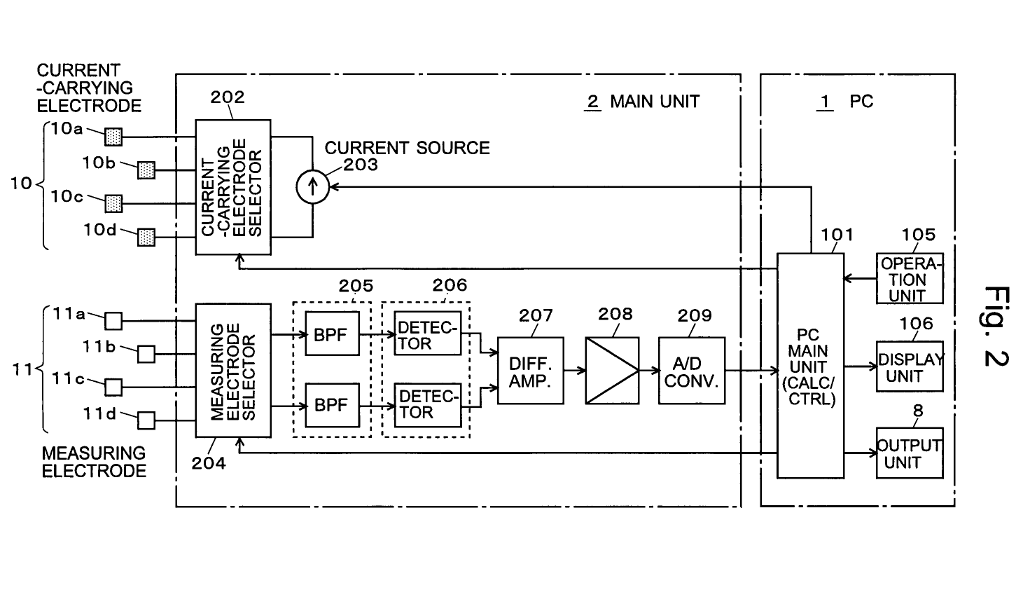

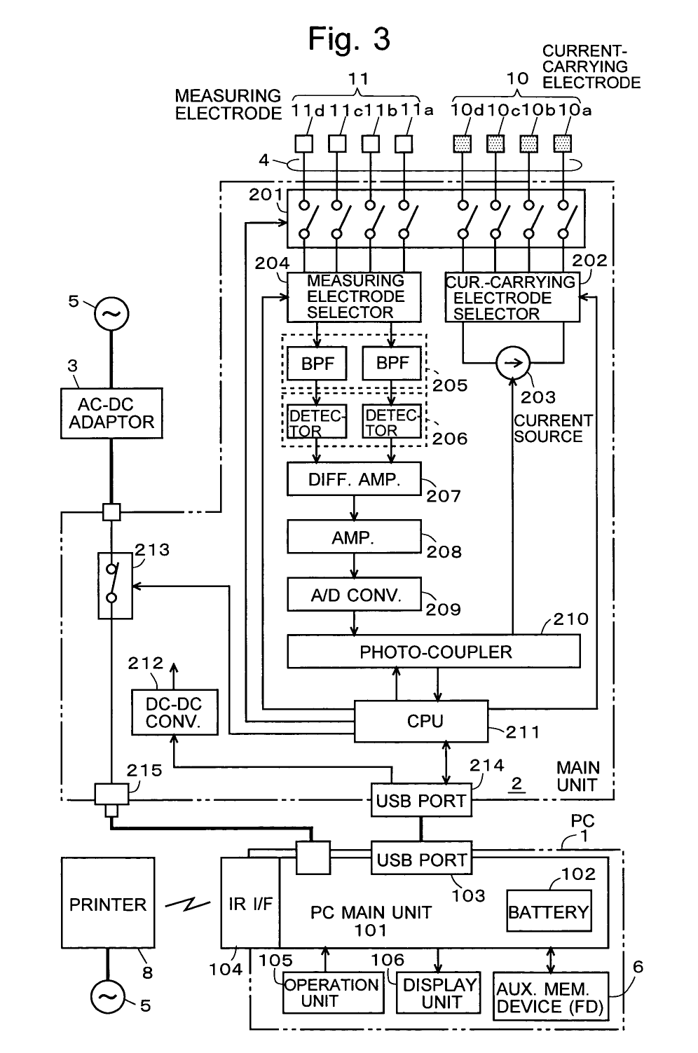

FIG. 2 is an outlined drawing of the electrical construction of the body composition measurement apparatus of the first embodiment, and FIG. 3 is a detailed drawing of the electrical construction. Four current-carrying electrodes 10a, 10b, 10c and 10d are connected via signal line opening/closing relays 201 to a current-carrying electrode selector 202, which selects two electrodes to be connected to a current source 203. The current source 203 generates a constant radio frequency current at frequency f₀, which is usually determined within the range 50 kHz to 150 kHz. Similarly, the four measuring electrodes 11a, 11b, 11c and 11d are connected via the signal line opening/closing relays 201 to a measuring electrode selector 204, which selects two electrodes and transfers the signals obtained with each of the two electrodes to each of band-pass filters (BPF) 205. The band-pass filters 205 remove component signals other than the frequency f₀. After that, detectors 206 detect and rectify the signals to extract component signals of frequency f₀. The signals detected in parallel are differentially amplified by a differential amplifier 207, and then further amplified by an amplifier 208. Analogue-to-digital converter (A/D) 209 converts the signals into digital signals, and sends the digital signals via a photo-coupler 210 to a central processing unit (CPU) 211. The CPU 211 is connected with the universal serial bus (USB) port 214 and has the function of the conversion/inversion of data for the USB interface. The CPU 211 not only sends to the USB terminal 211 the data corresponding to the output signal of the A/D converter 209, but also receives control signals through the USB port 214 and, based on the control signals, controls the operation of the current source 203 and the operations of the signal line opening/closing relays 201 and a power line opening/closing relay 213 (which will be mentioned later) through the photo-coupler 210. The optical connection between the CPU 211 and the analogue measurement circuits via the photo-coupler prevents the intrusion of digital noises generated by the CPU 211 or coming from the PC 1 into the analogue measurement circuits. The DC power output of the AC-DC adaptor 3 connected to a commercial AC power supply 5 is drawn into the main unit 2 and connected via the power line opening/closing relay 213 to a power output jack 215. The power cable for supplying electric power to the PC 1 is connected to the power output jack 205. Thus, the DC power output of the AC-DC adaptor 3 is connected to the PC 1, where the main unit 2 merely provides a pathway having only the power line opening/closing relay 213.

The PC 1 has a main unit 101 enclosing the CPU, read only memory (ROM), random access memory (RAM), hard disk drive, battery 102 and other components, an operation unit 105 including a keyboard and a pointing device, such as a mouse, and a display unit 106 composed of a liquid crystal display. Furthermore, the PC 1 has an infrared interface (I/F) 104 for connection with a printer 8. This construction includes no electrical connection by cables, so that the influence from the noises generated by the power supply of the printer 8 is eliminated. Furthermore, even if parts trouble or other trouble has occurred, the construction prevents an excessive current from the printer 8. Thus, such a case is assuredly avoided where an abnormal current flows through the body of the subject. The PC 1 has a standard USB port 103. As is generally known, USB interface is provided with lines for transferring serial data and DC power. In this embodiment, the USB port 103 of the PC 1 is capable of providing a maximum power output of 5V/50 mA. The main unit 2, connected with the PC 1 via a USB cable, receives the DC power from the PC 1 and distributes the power to each circuit through a DC-DC converter 212. Accordingly, every electrical circuit included in the main unit 2 is designed to operate by the power of 5V/500 mA or lower. In addition, the DC-DC converter 212 prevents the intrusion of noises through the power source into the analogue measurement circuit.

A calculation program for measuring the impedance and performing the calculations to estimate various kinds of information relating to the aforementioned body composition information and health condition based on the measurement value of the impedance, and a control program for conducting the measurement, are stored on the hard disk (or on the ROM) of the PC 1. In detail, first, MRI measurements are performed for a number of monitors with different body specific information including the height, weight, age, gender, etc. Then, from the results of the measurements, reliable regression analysis coefficients are derived to create an accurate estimation formula. The estimation formula is stored on the hard disk (or in the ROM) as a part of the calculation program. The program is executed in response to a command externally given through the operation unit 105, whereby the measurement of the impedance (to be described later) followed by calculation and display processes are practically performed. It should be noted that the estimation formula for calculation does not need to be stored in the form of an equation, but may be embodied in various forms. For example, it may be stored in the form of tables designed to receive measurement values of the impedances and the body specific information as input, and to give the body composition information and health information as output.

In the present body composition measurement apparatus, the signal line opening/closing relay 201 is provided for each cable 4 (i.e. signal path) connected to the current-carrying electrodes and the measuring electrodes, and the power line opening/closing relay 213 is provided for the power supply line connected to via the AC-DC adaptor 3 to the commercial AC power supply 5. The function of the signal line opening/closing relays 201 are to substantially disconnect the electrodes 10 and 11 from the main unit 2 at all times except for the period when the impedance of the body of the subject is measured. This prevents an undesirable current from flowing through the body of the subject when a trouble or malfunctioning of the circuits has occurred, thus ensuring the safety of the subject. The function of the power line opening/closing relay 213, on the other hand, is to disconnect the commercial AC power supply 5 from the main unit 2 and the PC 1 during the measurement of the impedance, to block the noises coming from the outside through the commercial AC power supply 5. This suppresses the noises during the measurement of the impedance, whereby the accuracy of the measurement is improved. Another function is to disconnect the commercial AC power supply 5 during the process of connecting the measurement circuits and the body via the electrodes 10 and 11 so that at least a leakage of the alternating current of 100V is prevented from entering the body when a trouble or malfunctioning of the circuits has occurred. This constitutes a dual safety measure with the signal line opening/closing relays 201.

In the body composition measurement apparatus of the first embodiment, the band-pass filters 205 and the detectors 206 are placed before the differential amplifier 207. This construction requires these circuits to be provided for each of the two input lines. Instead, the construction shown in FIG. 35 may be adopted, where the band-pass filter 205 and detector 206 are placed behind the differential amplifier 207. This construction is advantageous in that there is little influence from the noise, because the differential amplifier 207 cancels the common mode noise. The construction shown in FIG. 2 (and FIG. 3), on the other hand, is advantageous in that it reduces the measurement error, because it is hardly affected by the stray capacitance of the cables and the circuits, and it allows only a small phase rotation even when the two loads connected to the inputs of the band-pass filters 205 via the measuring electrodes is unbalanced.

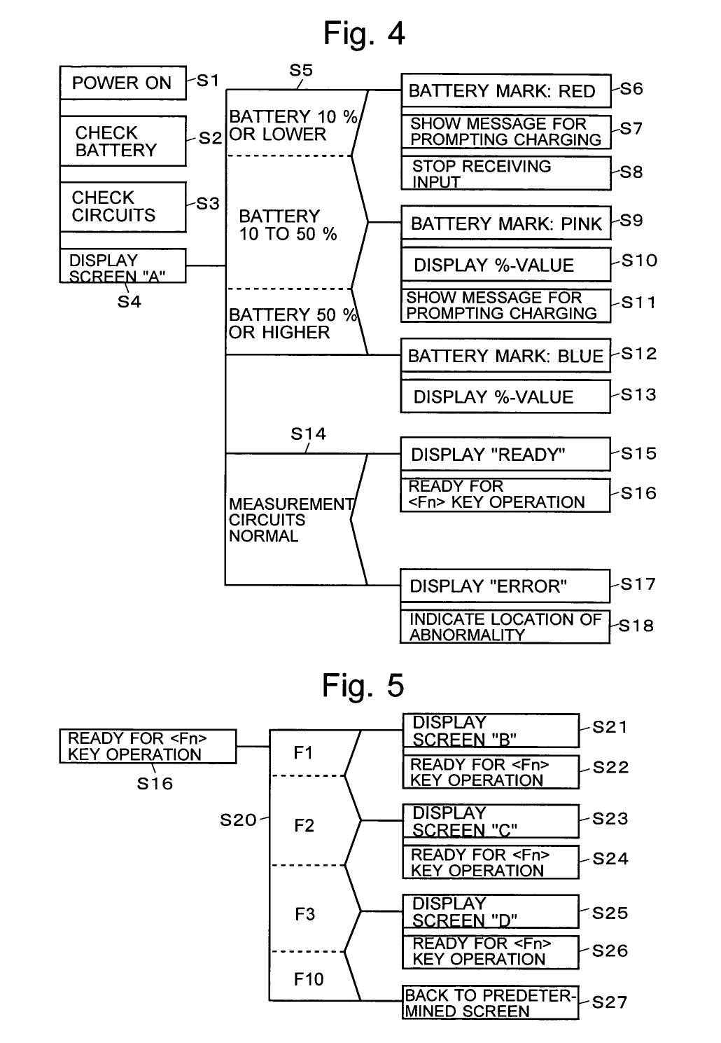

The actual measurement process using the above body composition measurement apparatus and the operation of the apparatus are described in detail. FIGS. 4 and 5 are problem analysis diagrams showing the initial operation of the measurement operation of the present body composition measurement apparatus.

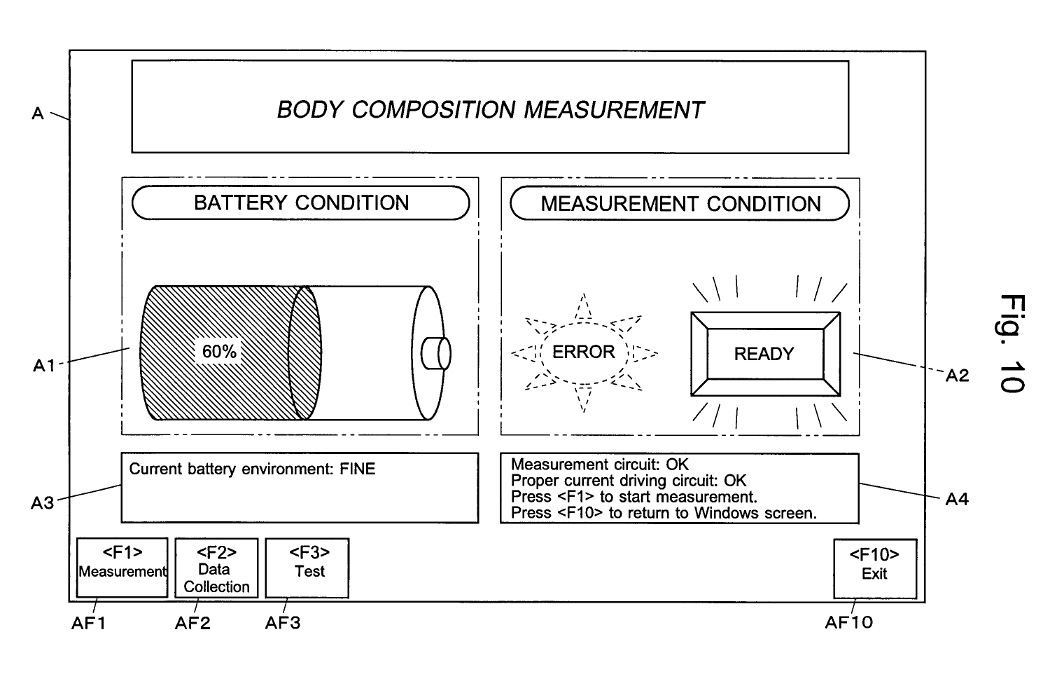

When the power switch of the PC 1 is turned ON (Step S1), the PC main unit 101 starts running, and performs the checking of the remaining power of the battery 102 (Step S2) and the checking of the measurement circuits (Step S3). Checking of the measurement circuits is a process performed according to a predetermined algorithm to check whether there is any trouble with the internal circuits. After the checking, a screen “A” as shown in FIG. 10 is displayed on the display unit 106 (Step S4). The screen A contains the following components: battery power indicator A1 with a battery mark image similar to a cell; measurement circuit check result display area A2 for showing the result of the checking of the measurement circuits;

message display areas A3 and A4 for showing text messages about the states of the remaining battery power and the measurement circuits, respectively; and function buttons AF1-AF3 and AF10. In the process of displaying the screen A, the following information is updated according the remaining power of the battery 102: percentage (%) of the remaining power and painted area of the battery mark image, shown in the battery power indicator Al; and content of the message shown in the message display area A3. When the remaining battery power is less than 10%, the painted area of the battery mark image becomes red (Step S6), and a message for prompting the charging of the battery is displayed (Step S7). In addition, the PC main unit 101 stops receiving any input relating to the measurement (Step S8). Thus, the battery is prevented from running out in the middle of the measurement. When the remaining battery power is 10 to 50%, the painted area of the battery mark image becomes pink (Step S9), and the remaining power is displayed by percentage (Step S10). In this case, also, the message for prompting the charging of the battery is displayed (Step S11), because the remaining power is not sufficient. When the remaining battery power is 50% or higher, the painted area becomes blue (Step S12), and the remaining power is displayed by percentage (Step S13). Thus, by looking at the display, the examiner can intuitively determine whether the remaining battery power is sufficient.

When the result of the measurement circuit check is “normal”, then “READY” is displayed in the measurement circuit check result display area A2 (Step S15), and the apparatus becomes ready for operation with the function buttons AF1-AF3 and AF10 enabled (Step S16). When, in contrast, the check result is “abnormal”, “ERROR” is displayed in the measurement circuit check result display area A2 (Step S17), and a message indicating the abnormal part is displayed in the message display area A4 (Step S18). In FIG. 10, “READY” is displayed in the measurement circuit check result display area A2; the “READY” will disappear when “ERROR” is displayed. From Step S8 or S18, the operation cannot automatically proceeds to Steps S15 and S16. For Step S8, the plug of the AC-DC adaptor 3 should be connected to a service outlet to start supplying the power. For Step S18, the examiner or other person should correct the abnormal part. On detecting these operations, the PC main unit 101 starts the process of Steps S15 and S16.

When screen A is displayed on the display unit 106, the examiner can select one of the function buttons AF1, AF2 and AF3 according to necessity by operating a mouse or other pointing device. The function buttons are associated with the function keys of the keyboard, so that the examiner can perform the same operation on the keyboard. To terminate the body composition measurement program, the examiner should select the function button AF10. In response to this operation, the PC main unit 101 terminates the body composition measurement program (application) and restores the screen of the display unit 106 to a predetermined state, e.g. the initial screen of the Windows 98, presented by Microsoft Corporation (Step S27).

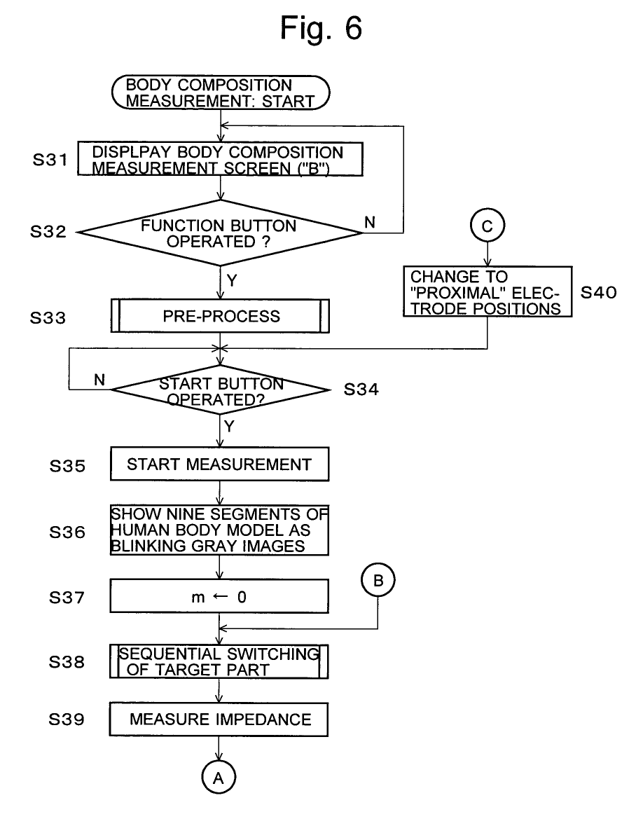

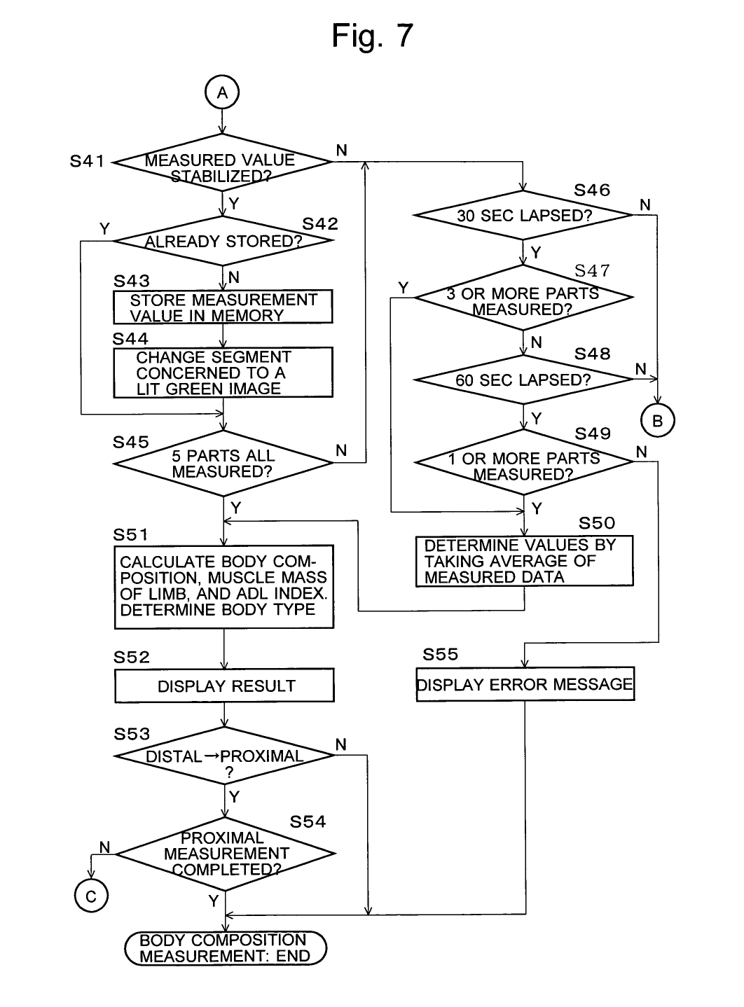

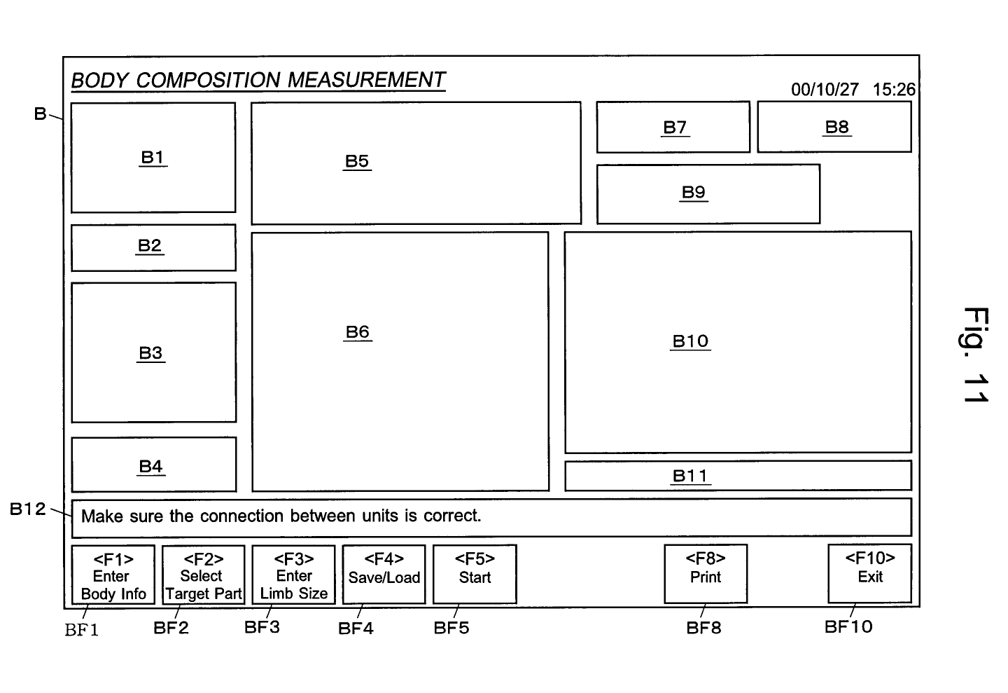

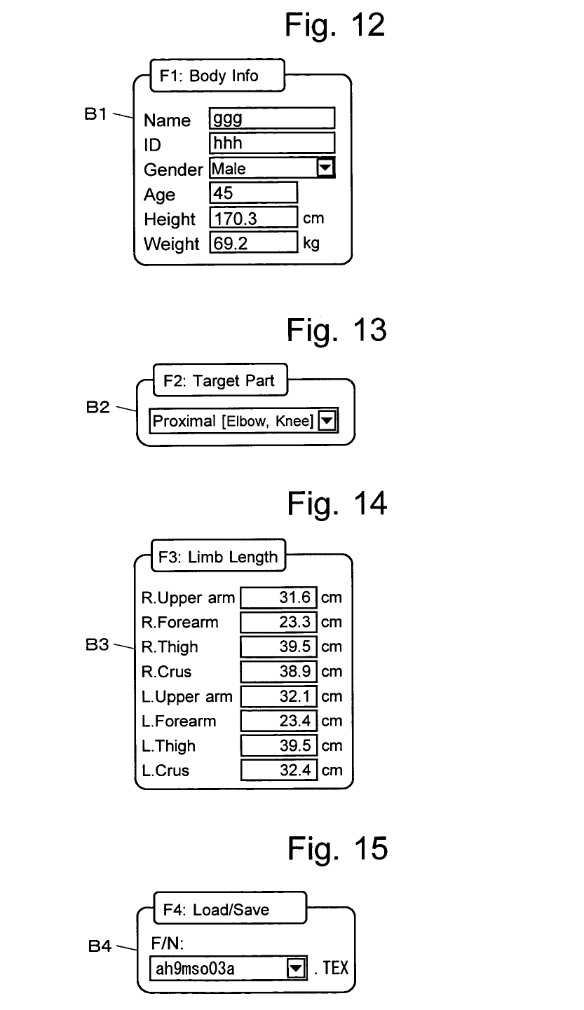

When screen A is displayed on the display unit 106, the function button AF1 is associated with a body composition measurement mode, which is a mode used for a normal measurement of the body composition. The function button AF2 is associated with a data collection mode, which is a mode intended to be used especially for research purposes. This mode provides very detailed options for measurements; for example, it is possible to select a body part and specify the sampling period to measure the temporal change in the impedance of the specified body part. The function button AF3 is associated with a test mode, which is a mode for correction of internal circuits or similar operation. Next, the operation in the body composition measurement mode is described referring to FIGS. 6-9 and 11-22. FIGS. 6-9 are the flowcharts and a problem analysis diagram of the operation in the body composition measurement mode, FIG. 11 is an outlined view of the screen displayed on the display unit 106 in the body composition measurement mode, and FIGS. 12-22 are detailed drawings of each part of the screen shown in FIG. 11.

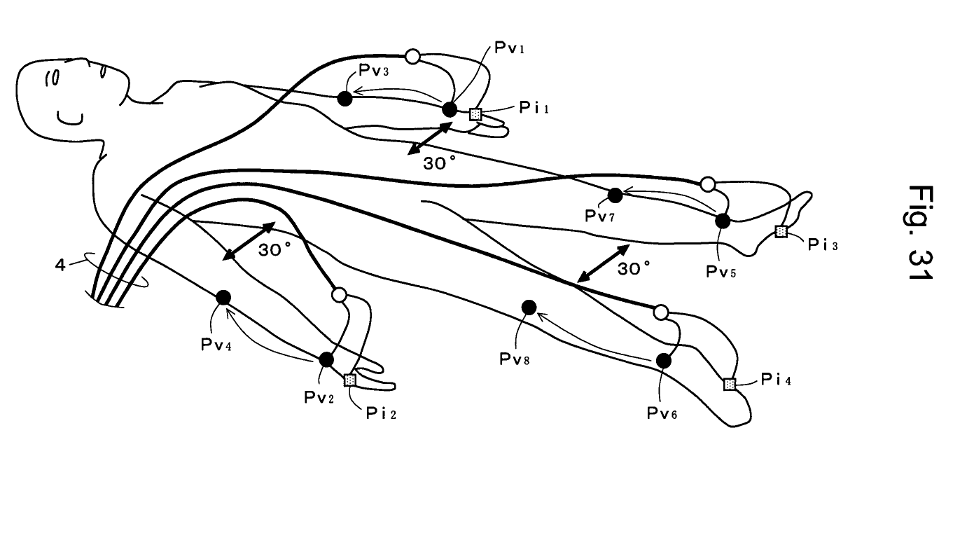

In the body composition measurement mode, the subject should lie on a bed or other similar surface. This position should be taken also in the data collection mode, which will be described later. FIG. 31 is a perspective view showing a recommended position for the measurement. As shown in FIG. 31, during the measurement, the subject should basically take a supine position on a bed or similar surface, with the four limbs fully extended and opened by the angle of about 30 degrees to keep the arms apart from the trunk and the legs apart from each other. To eliminate the influence from the change in the body fluid balance, it is preferable to keep the subject resting in the above position for about five minutes. Meanwhile, the examiner performs setting operations necessary for the measurement. That is, the examiner selects the function button AF1 on the initial screen A displayed on the display unit 106. In response to this operation, the PC main unit 101 replaces screen A with a body composition measurement screen B shown in FIGS. 11-22 (Step S31).

As shown in FIG. 11, the body composition measurement screen B contains the following components: body information display area B1, target part display area B2, limb length display area B3, file display area B4, electrode attachment position display area B5, measurement value display area B6, distal measurement value display area B7, proximal measurement result display area B8, ADL index display area B9, muscle mass display area B10, body type display area B11, message display area B12 and function buttons BF1-BF5, BF8 and BF10. The body information display area B1 has, as shown in FIG. 12, text boxes for entering and displaying the name and identifier (ID) of the subject and the body specific information, such as gender, age height and weight. The target part display area B2 has a text boxes for selecting “distal measurement”, “proximal measurement” or “distal measurement→proximal measurement”, as shown in FIG. 13. The limb length display area 3 has text boxes for entering and displaying the lengths of the upper arm, forearm, thigh and crus of each side of the body of the subject, as shown FIG. 14. When a value is entered in the “Height” text boxes, the lengths of the four limbs are automatically calculated from the height and displayed in the corresponding text boxes in the limb length display area B3. Therefore, the examiner does not have to manually enter the values as long as there is no need to change the automatically calculated values. The file display area B4 has a text boxes for entering and displaying the filename of a data file to be saved or read, as shown in FIG. 15.

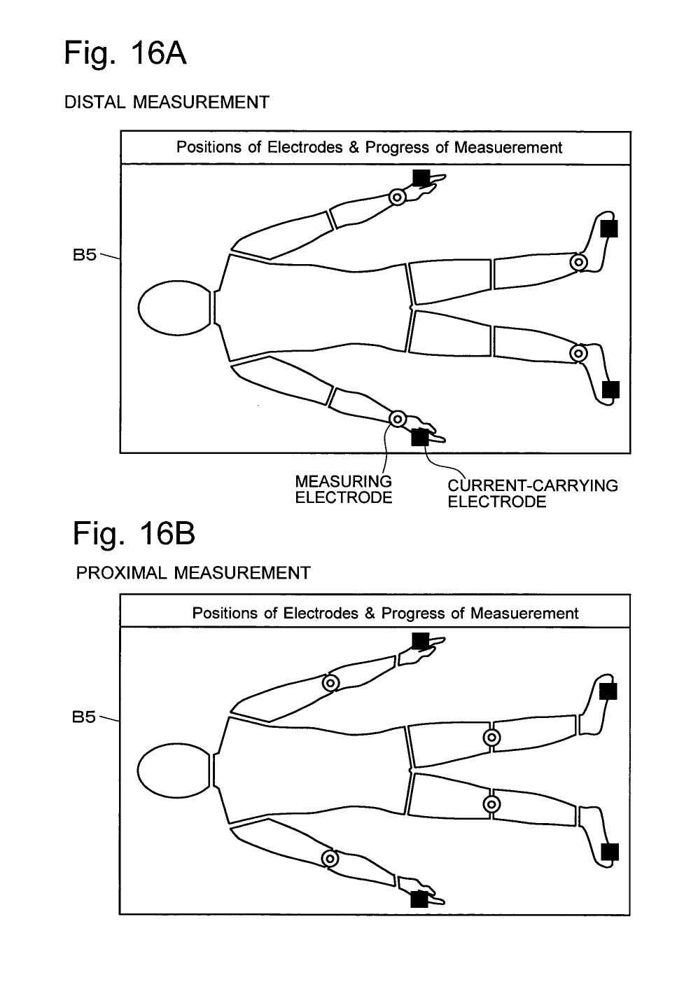

The electrode attachment position display area B5 shows a figurative model of the human body divided into nine segments, as shown in FIGS. 16A and 16B. On this model, the positions to attach the electrodes on the body are indicated by two symbols, where “▪” indicates the current-carrying electrode, and “⊚” indicates the measuring electrode. The attachment positions for the electrodes are determined corresponding to the measurement type selected in the target part display area B2. When “distal measurement” is selected, the symbols “⊚” of the measuring electrodes are displayed at both wrists and both ankles, as shown in FIG. 16A. When “proximal measurement” is selected, the same symbols are displayed at both elbows and both knees, as shown in FIG. 16B. When “distal measurement→proximal measurement” is selected, the symbols are displayed according to whether the distal or proximal measurement is to be performed next. By referencing the display area, the examiner can avoid attaching the current-carrying electrodes 10 and the measuring electrodes 11 to wrong positions. The color the figurative model of the body can be changed for each of the nine segments. When the measurement is started (as described later), the segment undergoing the measurement is displayed as a blinking gray image, which will turn into a lit green image after the completion of the measurement. Thus, it is possible to check the progress of the measurement by simply checking the visual state of the segments.

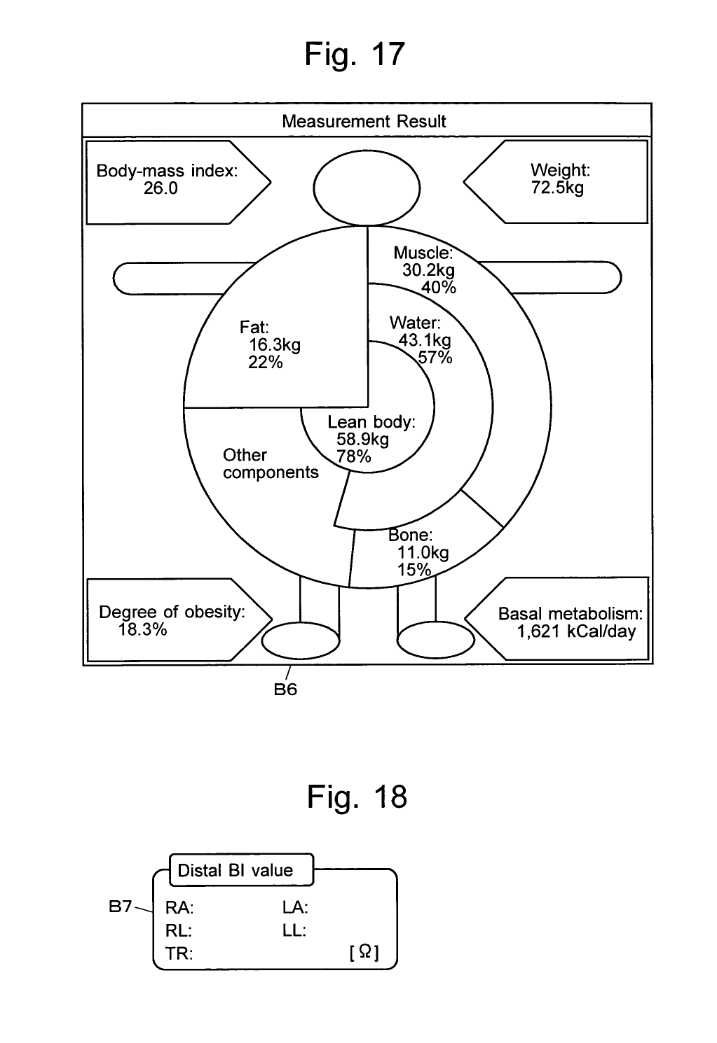

The measurement result display area B6 is an area for displaying the measurement result, where, as shown in FIG. 17, a circle chart representing the human body in a deformational way is displayed to show the following three types of body composition ratios: fat, muscle, bone and other components; fat and lean body; and fat, water and other components. In addition, the following information is displayed: weight; body-mass index (BMI), derived from the height and other body specific information; degree of obesity; and estimated value of the basal metabolic rate. The minimum unit of the percentage (%) shown inside the circle chart is 1%. The dividing lines inside the circle chart may be continuously changed according to the percentage values. In the present embodiment, however, the divisional lines are changed by the unit angle of a quarter to one sixteenth of full circle (360 degrees), i.e. by the step of 22.5 to 90 degrees. This method simplifies the graphic display process, so that the chart can be quickly drawn.

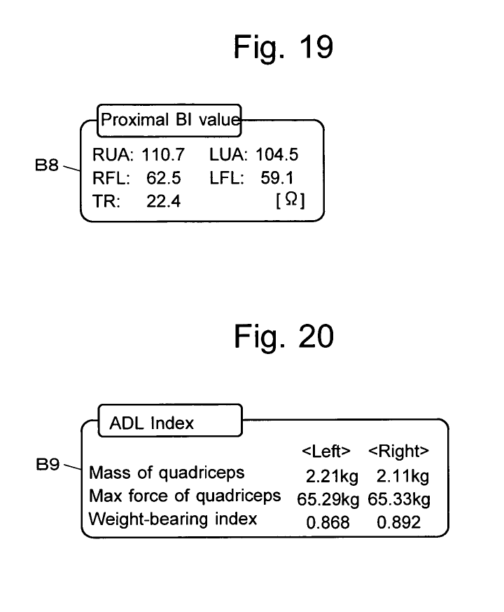

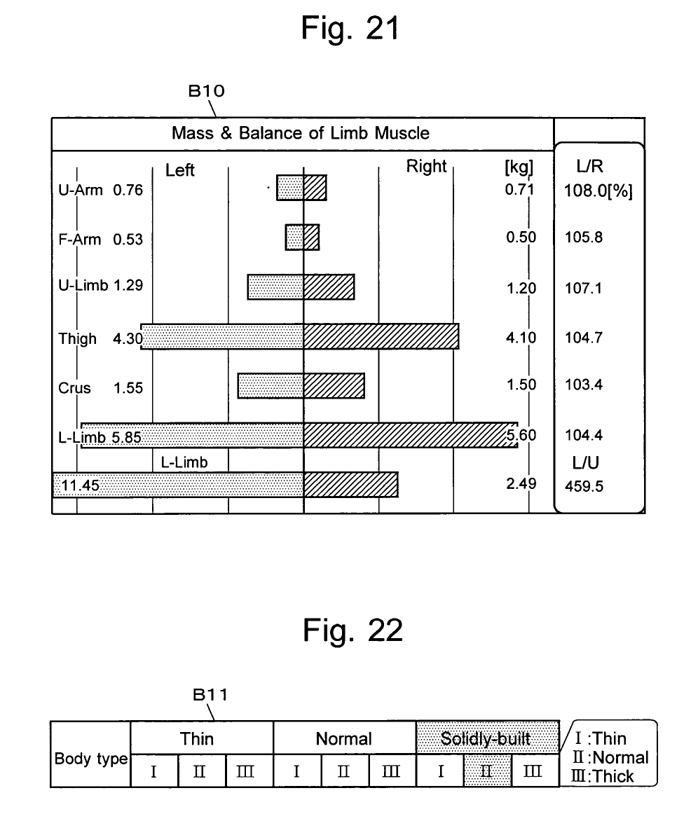

The distal measurement value display area B7 and the proximal measurement value display area B8 display the measurement value of the impedance of each segment, as shown in FIGS. 18 and 19. The ADL index display area B9 displays, as shown in FIG. 20, the masses and maximum forces of the right and left quadricepses, and the right and left weight bearing indices, all estimated from the measurement result. These values serve as the ADL indices for evaluating the ability for the activity of daily living. The muscle mass display area B10 displays, as shown in FIG. 21, a bar chart showing the estimated masses of the upper arms, forearms, arms, thighs, crura and legs on the right and left sides. In addition, the percentages of the right-and-left muscle masses are displayed to show the right-and-left balance. The ratio of the muscle mass of the arms to that of the legs is also displayed. These items of information make it easy to visually recognize the balance between the right and left muscles, from which it is possible to know, for example, which is the dominant arm or leg. The information can be used also for instant health examination. For example, when the right-and-left balance is abnormal, it is possible to guess that something is wrong with the health condition. The body type display area B11 displays, as shown in FIG. 22, the external body type as “thin”, “normal” or “solidly-built”, according to the body-mass index (BMI: W/H²) calculated from the weight and height entered as the body specific information. Furthermore, based on the body-fat ratio obtained as a result of the measurement, the state of deposits in the fat is displayed as “thin”, “normal” or “thick”. These categories are different from the aforementioned external body types; they represent the body type based on the state of the body composition.

The message display area B12 displays messages that the examiner (or the subject) should know in the course of the measurement, as shown in FIG. 11. In addition, seven function buttons BF1-BF5, BF8 and BF10 are displayed below the message displaying area B12. Function buttons BF1-BF4 are associated with the function of activating the body information display area B1, target part display area B2, limb length display area B3 and file display area B4 to allow data input or deactivating these areas to fix the input data. Function button BF5 is associated with the function of giving commands for starting/stopping (or pausing) the measurement. Function button BF8 is associated with the function of giving a command for printing. Function button BF10 is associated with the function of discontinuing the body composition measurement mode and returning to the initial screen A.

Back to FIG. 6, the PC main unit 101 is now ready for operation with the function buttons activated for selection (Steps S31, S32). When one of the function buttons BF1-BF4 is selected, a pre-process for the measurement is performed according to the selection (Step S33).

FIG. 8 is a process analysis diagram showing the pre-process for the measurement. When function button BF1 is selected, the PC main unit 101 sets a blinking cursor in one of the text boxes in the body information display area B1 to indicate what item of information should be entered. Using the keyboard, the examiner enters the name, ID number and body specific information of the subject, such as the gender (Sx), age (Ag), height (H) and weight (W) (Step S82). The main unit 101 is designed to not accept the command for starting the measurement without the entry of the body specific information. When the height H is entered, the PC main unit 101 estimates the lengths of four limbs by predetermined formulae (Step S83). For example, the length of the left upper arm is given by:

L_{LUA}=a_{LUA}×H+b_{LUA},

where a_{LUA} and b_{LUA} are constants. Lengths of the other segments can be similarly calculated. The estimated values are displayed in the text boxes of the limb length display area B3 (Step S84). These values become the default values for the lengths of the four limbs corresponding to the height entered. When the function button BF1 is selected again (Step S81), the main unit 101 discontinues the state of allowing the entry of the body information, and the entered information is fixed.

If there is no need to modify the estimated values of the lengths of the limbs, the default values will be used in the calculation of body composition, which will be described later. In most cases, the body composition can be estimated with considerably high accuracy by using the default values. However, it may be desirable to have higher measurement accuracy, or the subject may have a special body build, like athletes who often have one or more limbs abnormally developed depending on the kind of sport activity. In such a case, it is preferable to measure the actual lengths of the limbs and enter the measurement values in the limb length display area B3. To do this, the “limb length entry” function button BF3 should be selected in Step S80. In response to this selection, the PC main unit 101 sets a blinking cursor in one of the text boxes of the limb length display area B3, allowing the value in the text boxes to be changed. There, the examiner directly enters the measurement value, where the displayed value is changed on the spot (Steps S91, S92). When the function button BF3 is selected again (Step S90), the main unit 101 discontinues the state of allowing the entry of the lengths of the limbs, and the entered information is fixed.

In Step S80, when the “target part selection” function button BF2 is selected, the PC main unit 101 activates the text boxes of the target part display area B2 to allow the selection of the measurement method (Step S86). To perform the measurement for the aforementioned nine segments, the examiner should select the “distal measurement→proximal measurement”. Then, as shown in FIG. 16A, the symbols “⊚” indicating the measuring electrodes are displayed on the figurative model of the human body at both wrists and both ankles, and the symbols “▪” indicating the current-carrying electrodes are displayed at the backs of the both hands and at the insteps of both feet (Step S89). This arrangement is displayed also when the “distal measurement” is selected (Step S87). When, on the other hand, the “proximal measurement” is selected, the symbols “⊚” indicating the measuring electrodes are displayed on the figurative model of the human body at both elbows and both knees, as shown in FIG. 16B, while the symbols “▪” indicating the current-carrying electrodes are displayed at the same positions (Step S88). When the function button BF2 is selected again (Step S85), the main unit 101 discontinues the state of allowing the selection of the target part, and the entered information is fixed.

It is assumed here that the “distal measurement→proximal measurement” has been selected. Then, the symbols “▪” are displayed at the backs of the right and left hands and at the insteps of right and left feet, and the symbols “⊚” are displayed at the right and left wrists and ankles. Referencing the displayed image, the examiner affixes the current-carrying electrodes 10 to the back or instep of the right and left hands and feet at a point in the vicinity of the root of the middle finger, and the measuring electrodes 11 to the right and left wrists and ankles. Thus completing the preparation for the measurement, the examiner operates the “start” function button BF5 to give a command for starting the measurement (Step S34). In response to this operation, the PC main unit 101 starts the measurement (Step S35). First, in the figurative model of the human body shown in the electrode attachment position display area B5, all the segments selected as the targets of the measurement are displayed as blinking gray images (Step S36), and a variable for switching the electrodes, m, is set to zero (Step S37). After that, a process of sequentially switching the target part is performed (Step S38).

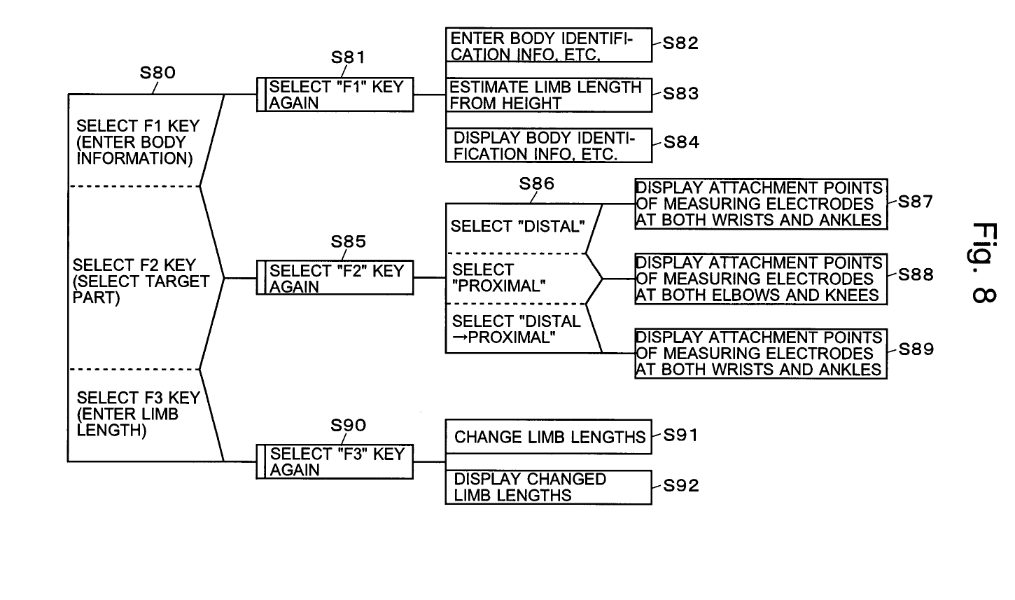

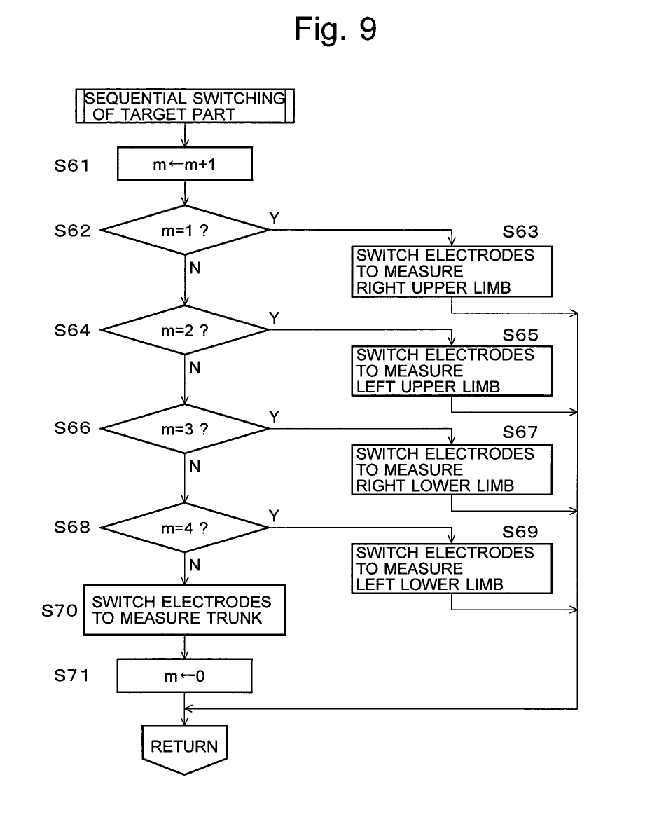

FIG. 9 is a detailed flow chart showing the process of sequentially switching the target part. First, the variable m is incremented by one (Step S61), and then it is checked whether m is equal to one of four values 1, 2, 3 and 4 (Steps S62, S64, S66 and S68). When m=1, the connections of the current-carrying electrode selector 202 and the measuring electrode selector 204 are controlled so that the right arm is selected as the target part (Step S63). When m=2, 3 or 4, the connections of the current-carrying electrode selector 202 and the measuring electrode selector 204 are controlled so that the left arm, right leg or left leg is selected as the target part, respectively (Steps S65, S67, S69). In Step S68, when m is not equal to 4, the connections of the current-carrying electrode selector 202 and the measuring electrode selector 204 are controlled so that the trunk is selected as the target part (Step S70), and m is reset to zero (Step S71). After the connections are changed according to the target part selected, the operation proceeds to Step S39, where the impedance is measured. By the process of sequentially switching the target part as described above, the connections of the electrodes 10 and 11 are changed so that the measurement is sequentially performed for the right arm, left arm, right leg, left leg and trunk in this order. Immediately after the start of the measurement, the connections of the electrodes 10 and 11 are changed so that the measurement is performed for the right arm, which is equivalent to the segment including the right upper arm and the right forearm. After that, a constant current is supplied from the current source 203 between the two current-carrying electrodes 10, and the potential difference generated by the current is measured with the two measuring electrodes 11. The measurement signals are transferred via the BPF 205 and the detector 206 to the differential amplifier 207.

The PC main unit 101 reads the digitized voltage value once every sampling period of the A/D converter 209, calculates the impedance from the voltage value and the current value, and determines whether the measurement value of the impedance has been stabilized (Step S41). In this process, the change of the measurement value per unit time is calculated from the measurement values sequentially obtained. When the state where the change is equal to or less than 1 [Ω/sec] has continuously occurred a predetermined number of times, the measurement value is determined as stabilized. When the measurement value is determined as stabilized, it is checked whether the measurement value has already been stored (Step S42), and if not, the measurement value is stored in the internal memory (Step S43). After that, in the electrode attachment position display area B5, the displayed state of the corresponding segment (i.e. combination of the right upper arm and the right forearm in this case) of the figurative model of the human body is changed from the blinking gray image to a lit green image (Step S44). Thus, the examiner can visually check the progress of the measurement. Since the measurement value is stored in the memory after it is stabilized, the accuracy of the measurement value of the impedance is improved.

After that, it is checked whether the measurement has been completed for all of the five target parts, i.e. the four limbs and the trunk (Step S45). If any part remains unmeasured, the operation proceeds to Step 46. Similarly, when, in Step S41, the measurement value is determined as not yet stabilized, the operation proceeds to Step S46. In Step S46, it is checked whether thirty seconds have passed since the start of the measurement. When thirty seconds have not yet passed, the operation returns to Step S38 to continue the measurement. When thirty seconds have passed, it is checked whether the measurement has already been completed for three or more of the five target parts (Step S47). When the measurement has already been completed for three or more target parts, the measurement values of the unmeasured parts are determined by taking the average of the measurement values already obtained, and the values are stored in the memory (Step S50). When, in Step S47, the measurement has not yet completed for three or more target parts, it is checked whether sixty seconds have passed since the start of the measurement (Step S48). When sixty seconds have not yet passed, the operation returns to Step S38 to continue the measurement. When sixty seconds have passed, it is checked whether the measurement has already been completed for one or more of the five target parts (Step S49). When the measurement has been completed for one or more target parts, the process of Step S50 is performed. When, in Step S49, the measurement has not yet been completed for one or more target parts, it means that the measurement value is not yet stabilized in any target part even after the lapse of sixty seconds. In this case, it can be concluded that there is some abnormality in the measurement. Therefore, an error message, such as “Measurement not performable” or “Operation abnormal”, is displayed in the message display area B12 of the body composition measurement screen B (Step S55), and the measurement is terminated.

The process of Steps S41-S50 prevents the measurement from taking an abnormally long time because of an unstable measurement state. That is, at the moment the measurement has been continued for a certain period of time, if the measurement has been completed for some target parts, the values of the unmeasured part are estimated from the measured data of the other parts, and the measurement of the impedance itself is terminated. This operation prevents the subject from being overburdened.

When, in Step S45, it is determined that all the measurements are completed, or when the process of Step S50 has been performed, the memory holds the measurement values of the impedances corresponding to the five target parts (right arm, left arm, right leg, left leg and trunk, in the case of distal measurement). From these measurement values of the impedances and the body specific information, the PC main unit 101 calculates the body composition, the muscle mass of the quadriceps and the ADL indices, and determines the body type, using the above-described estimation methods (Step S51). When only the distal measurement has been completed, it is impossible to perform a detailed estimation in which each arm is divided into upper arm and forearm and each leg is divided into thigh and crus. Accordingly, for these segments, values are roughly estimated from the body specific information and other information. The above calculations provide all the results that should be displayed in the measurement result display area B6, distal measurement value display area B7, ADL index display area B9, muscle mass display area B10 and body type display area B11 of the body composition measurement screen B; the results are displayed on the display unit 106 (Step S52).

Next, it is checked whether “distal measurement→proximal measurement” is selected as the selection of the target part (Step S53). When the “distal measurement→proximal measurement” is selected, then it is determined whether the proximal measurement has been completed (Step S54). If the “distal measurement→proximal measurement” is selected and the proximal measurement has not been completed, the attachment positions of the measuring electrodes 11 on the figurative model of the human body displayed in the electrode attachment position display area B5 are moved from the distal positions to the proximal positions (Step S40). In concrete the symbols that have been displayed at the right and left wrists and ankles are moved to the right and left elbows and knees. After that, the operation returns to Step S34, and the PC main unit 101 waits for the “start” function button BF5 to be selected again. Meanwhile, referencing the displayed image, the examiner changes the attachment positions of the four measuring electrodes 11 to the right and left elbows and knees of the subject, and operates the “start” function button BF5 again to give a command for restarting the measurement. After that, the proximal measurement of the four limbs and the trunk is performed in the same way as described above.

After the proximal measurement of the four limbs and the trunk is completed, the operation proceeds through Steps S45, S51, S52, S53 and S54 in this order. This time, the results of both the distal and proximal measurements are completely available, so that the measurement values corresponding to the nine segments can be obtained. Therefore, in Step S51, the body composition information and other kinds of information are obtained more accurately than in the case of the distal measurement. In Step S52, newly measured values are displayed in the proximal measurement value display area B8 of the body composition measurement screen B, and the values displayed in the measurement result display area B6, ADL index display area B9, muscle mass display area B10 and body type display area B11 are replaced with newly calculated values. After that, the operation proceeds through Steps S53 and S54 to the end.

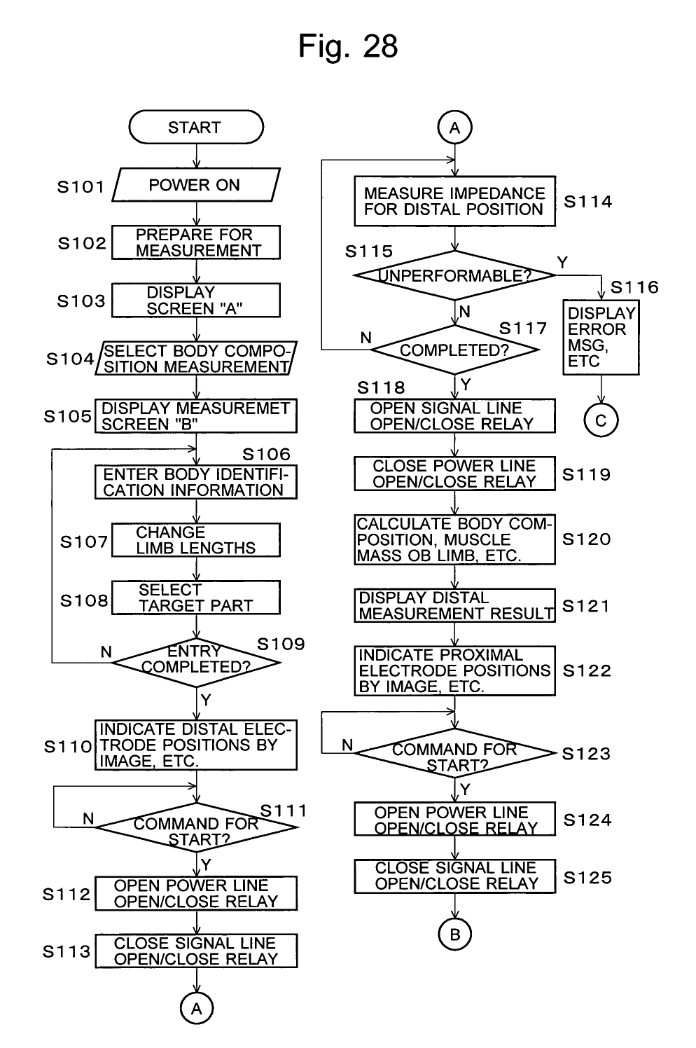

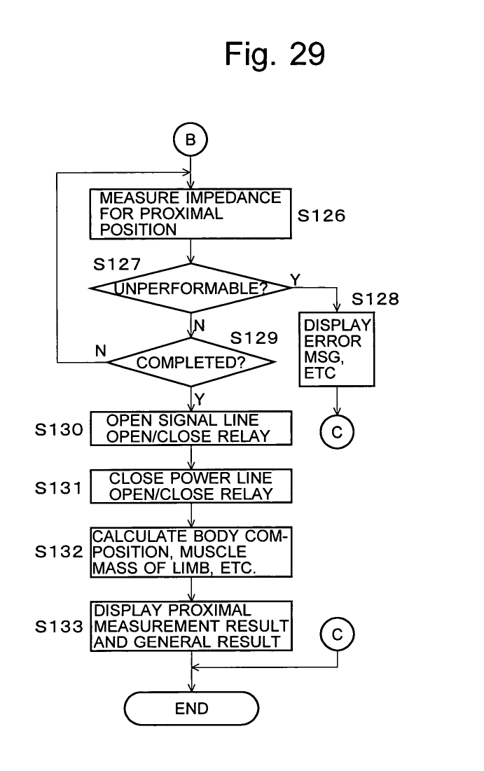

FIGS. 28 and 29 are flowcharts plainly showing the control flow of the operation of the present body composition measurement apparatus, focusing on the measurement of the impedance f6r each of the nine segments and on the estimation of the body composition information using the measurement values of the impedances. Including some repetitions of the above descriptions, the measurement operation is described along with the flowcharts.

When the examiner or other person turns on the power switch of the PC 1 (Step S101), the PC 1 starts running and performs a measurement preparation process including initialization, checking of the remaining power of the battery 102, self-checking of the measurement circuits, etc (Step S102). After the measurement preparation process, an initial screen “A” as shown in FIG. 10 is displayed on the display unit 106 (Step S103). The initial screen A has a battery power indicator Al and a message display area A3, which show the remaining battery power by the area and color of the painted part of the battery mark image, the numerical value, etc. When the remaining battery power is not sufficient, a message for prompting the charging of the battery or similar message is displayed. The initial screen A also includes a measurement circuit check result display area A2 and a message display area A4, which provide information about whether any abnormality has been detected by the measurement circuit check and, if any, the location of the abnormality.

The operation is allowed to proceed to the subsequent measurement process only when the remaining power of the battery 102 is higher than predetermined (e.g. 10% or higher) and the measurement circuits are normal. For example, when the remaining power of the battery 102 is insufficient, the plug of the AC-DC adaptor 3 should be connected to the socket of the commercial AC power supply 5, or when there is an abnormality in the measurement circuits, the abnormality should be duly eliminated. After that, the operation is allowed to proceed to Step S104. When the remaining power of the battery 102 is higher than predetermined and the measurement circuits are normal, if the examiner selects the function button A5 on the screen A with a mouse or other pointing device, or operates the keyboard to perform an operation having the same function (Step S104), then the operation enters the body composition measurement mode, and the initial screen A on the display unit 106 is replaced with the body composition measurement screen B (Step S105).

With the body composition measurement screen B displayed on the display unit 106, when the examiner selects the function button B12, a blinking cursor appears in the body information display area B1 having text boxes for entering and displaying the name and identifier (ID) of the subject and body specific information, such as gender, age, height and weight, to indicate what item should be entered. Looking at the screen, the examiner operates the keyboard to enter the name, ID and body specific information of the subject (Step S106). When the height is entered, the lengths of the four limbs are calculated by predetermined formulae, the result of which is displayed in the text boxes of the limb length display area B3. When, for example, it is desired to enter the actually measured lengths of the limbs of the subject, the function button B14 should be selected. Then, a blinking cursor appears in a text boxes of the limb length display area B3, indicating what item should be entered. There, the value can be changed (Step S107). When the values are not changed, the values calculated as described above are used as the lengths of the limbs in the calculation, which will be described later.

Next, the examiner selects the function button B13 (for selection of target part) to select “distal”, “proximal” or “distal→proximal” in the text boxes of the target part display area B2. It is assumed here that the “distal→proximal” is selected in order to measure the nine segments. It is of course possible to select “distal” or “proximal”. When all the body specific information has been entered, it is determined that the entry is completed (“Yes” in Step S109), and the electrode attachment positions for the distal measurement are indicated in the electrode attachment position display area B5 (Step S110). As explained above, the electrode attachment position display area B5 shows a figurative model of the human body divided into nine segments exclusive of the head and the fingertips of the hands and the feet, on which the positions to attach the electrodes are indicated by the two symbols: “▪” for the current-carrying electrode 10 and “⊚” for the measuring electrode 11. Referencing the displayed image, the examiner attaches the current-carrying electrodes 10 and the measuring electrodes 11 to the body of the subject.

After all the electrodes 10 and 11 have been attached to the body, the examiner operates the “start” function button B15 to give a command for starting the measurement (Step S111). In response to this operation, the measurement is automatically started. In advance of the measurement, the power line opening/closing relay 213 is opened (Step S112) and, slightly later than that, the signal opening/closing relays 201 are closed (Step S113). As a result, the commercial AC power supply 5 is disconnected from the main unit 2, and then the electrodes and 11 are connected to the main unit 2. Therefore, if any trouble should happen, there is no possibility that the alternating current from the commercial AC power supply 5 enters the body of the subject. Also, during the subsequent measurement period, the entry of noises from the commercial AC power supply 5 can be prevented.

After that, the current-carrying electrodes 10 and the measuring electrodes 11 are switched by the current-carrying electrode selector 202 and the measuring electrode selector 204 so that the right arm, left arm, right leg, left leg and trunk are sequentially selected as the target part. A weak radio frequency current is supplied between two current-carrying electrodes 10 selected, and the electric potential generated by the current is measured one after another with two measuring electrodes 11. In the figurative model of the human body displayed in the electrode attachment position display area B5, all the segments selected as the target parts are shown as blinking gray images before the measurement. After that, segments for which the measurement has been completed are changed to lit green images. This makes it possible to know the progress of the measurement by simply looking at the displayed image.

In the measurement of the impedance of each target part, the measurement value is not stored into the memory until the impedance is stabilized to a certain extent. If the measurement value remains unstable for a long time and the measurement is not completed for any of the target parts even after a predetermined period of time, it is determined that the measurement is not performable (Step S115). When the measurement is completed for all of the five target parts, or when the measurement is completed for at least one target part after a predetermined period of time, the measurement is determined as completed (Step S117). In the case of determining that the measurement is not performable, it is probable that there is some abnormality in the measurement. Accordingly, an error message, such as “Measurement not performable” or “Operation abnormal”, is displayed in the message display area B112 (Step S116), and the measurement is terminated.

The process of Step S15 prevents the measurement from taking an abnormally long time because of an unstable measurement state. That is, at the moment the measurement has been continued for a certain period of time, if the measurement has been completed for some target parts, the values of the unmeasured parts are estimated from the measured data, and the measurement of the impedance itself is terminated. This operation prevents the subject from being overburdened.

After the measurement is completed, the signal line opening/closing relays 201 are opened (Step S118) to disconnect the electrodes 10 and 11 from the main unit 2. After that, the power line opening/closing relay 213 is closed (Step S119) to connect the AC-DC adaptor 3, which is connected to the commercial AC power supply 5, to the main unit 2. Therefore, the electrodes 10 and 11 are connected to the measurement circuits only during the measurement of the impedance, that is, only for a short period of time including a period of time for supplying the current into the body of the subject and measuring the voltage generated by the current. During the measurement of the impedance, the commercial AC power supply 5 is disconnected, and the main unit 2 and the PC 1 run on the DC power supplied from the battery 102. After that, the impedances corresponding to the five target parts (right arm, left arm, right leg, left leg and trunk, in the case of distal measurement) and the body specific information are applied to predetermined estimation formulae, or to conversion tables corresponding to the formulae, to calculate the body composition, muscle masses of the limbs, ADL indices and body type (Step S120). This calculation may be performed using estimation formulae created using the body composition information obtained by the above-described MRI method. The estimation method is not always limited to this one. When only the distal measurement has been completed, it is impossible to perform a detailed estimation in which each arm is divided into upper arm and forearm and each leg is divided into thigh and crus. Accordingly, for these segments, values are roughly estimated from the body specific information and other information.

The numeral values obtained by the above estimation process are displayed in the measurement result display area B6, measurement value display area B7, ADL index display area B9, muscle mass display area B10 and body type display area B11 of the body composition measurement screen B, as described above (Step S121). After the distal measurement is completed, some items of information that can be estimated at the moment can be displayed, even though both distal and proximal measurements are not completed.

When the distal measurement is completed, the attachment positions for the measuring electrodes 11 displayed on the figurative model of the human body in the electrode attachment position display area B5 are moved to the proximal positions shown in FIG. 16B (Step S122). That is, the symbols displayed at the right and left wrists and both ankles are moved to the right and left elbows and knees. Checking the change of the screen image, the examiner moves the four measuring electrodes 11 to the right and left elbows and ankles, and then operates the “start” function button B15 to give a command for starting the measurement (Step S123). After that, the operation proceeds through Steps S124-S131, which correspond to Steps S113-S119 in the distal measurement, to perform the proximal measurement of the impedance of the limbs and the trunk. Now, the results of the distal and proximal measurements are completely available, so that the measurement values of the impedances corresponding to the nine segments can be obtained. Therefore, in the calculation of Step S132, the body composition and other information are estimated more accurately than when only the distal measurement has been completed. Then, the values thus calculated are displayed in place of the values shown in the measurement value display area B7, measurement result display area B6, ADL index display area B8, muscle mass display area B9 and body type display are B10 of the body composition measurement screen B (Step S133), and the measurement is terminated.

Thus, the present body composition measurement apparatus makes it possible to obtain accurate information reflecting the body composition and/or health condition in a relatively short period of time. Therefore, the subject experiences only a little physical or mental burden. Though the examiner must rearrange the electrodes in the course of the measurement, the work or operation is neither difficult nor complicated; the examiner has only to determine the attachment positions as instructed on the screen. Thus, the measurement can be easily performed. The information obtained by the measurement not only includes the body composition information, such as body fat mass or muscle mass, but also other information reflecting the health condition, such as the ADL index or the balance in muscle mass between the right and left side or upper and lower halves of the body. The information obtained thereby can be effectively used for various purposes, such as health management, physical training or rehabilitation.

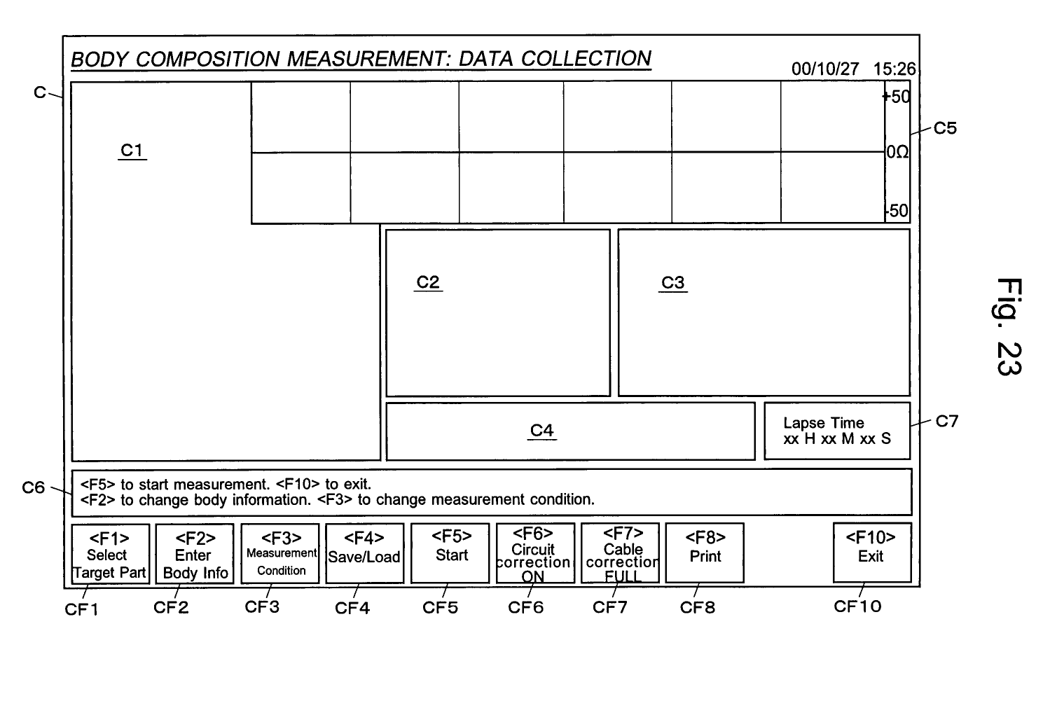

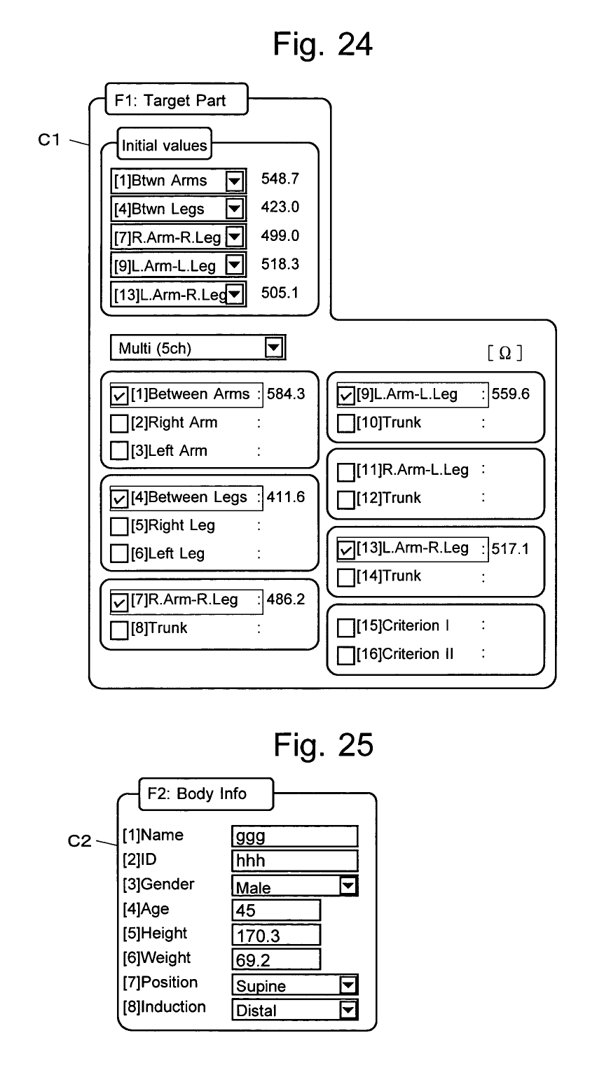

For normal purposes, such as health management, the body composition measurement mode can provide satisfactory results. In addition, the present body composition measurement apparatus is provided with the aforementioned data collection mode for collecting more detailed body composition information mainly for research purposes. To perform the measurement in the data collection mode, the function button AF2 should be selected when the screen A is displayed on the display unit 106. In response to this operation, the PC main unit 101 replaces the screen A with a data collection screen C shown in FIG. 23. Detailed parts of the data collection screen C are shown in FIGS. 24-27.

As shown in FIG. 23, the data collection screen C contains the following components: target part display area C1, body information display area C2, measurement condition display area C3, file display area C4, chart display area C5, message display area C6 and function buttons CF1-CF8 and CF10. The file display area C4, chart display area C5, message display area C6 and principal function buttons CF1-CF8 and CF10 are the same as the corresponding components having the same names in the body composition measurement mode. Accordingly, the description is omitted here. As shown in FIG. 24, the target part display area C1 shows the target parts with the impedance values obtained as the results of the measurement. In the data collection mode, in order to perform the measurement over a period of time arbitrarily determined as will be described later, the initial values of the impedances corresponding to five line charts displayed in the chart display area C5 are displayed in the upper area, and the measurement values of the impedances at each time point are displayed in the lower area. Details of the target parts will be described later.

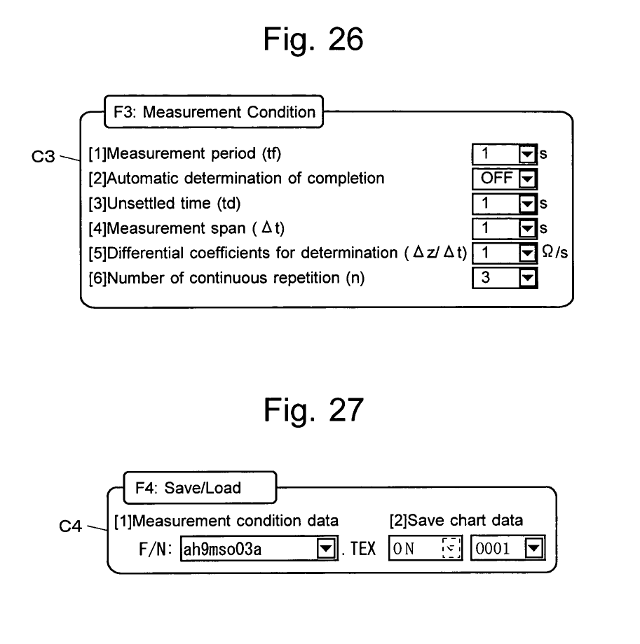

The body information display area C2 has, as shown in FIG. 25, text boxes for entering and displaying the name, identifier (ID) and body specific information (such as gender, age, height and weight) of the subject, and the position of the subject and the body parts for induction (or measurement). The measurement condition display area C3 has, as shown in FIG. 26, text boxes for entering and setting measurement parameters, such as the measurement period, use of automatic determination of the completion, unsettled time, measurement span, differential coefficients for determination and number of continuous repetitions. Detailed descriptions for there components are omitted here. Appropriate settings of these parameters will make it possible to obtain detailed data used particularly for research purposes. As shown in FIG. 27, the chart display area CS shows the change of the impedance with time during the measurement by the lines drawn in different colors for different segments. The vertical scale of the chart can be selected from four settings: ±5, ±10, ±20 and ±50, where ±10 is the initial setting. Vertical scrolling is also available. These features make it possible to easily compare the lines showing plural results. The message display area C6 displays various messages that the examiner (or the subject) should know in the course of the measurement. In addition, nine function buttons CF1-CF8 and CF10 are displayed below the message displaying area C6. The function buttons CF1-CF5, CF8 and CF10 correspond to the function buttons BF1-BF5, BF8 and BF10, respectively. The lapse time display area C7 displays the lapse time from the start of the measurement.

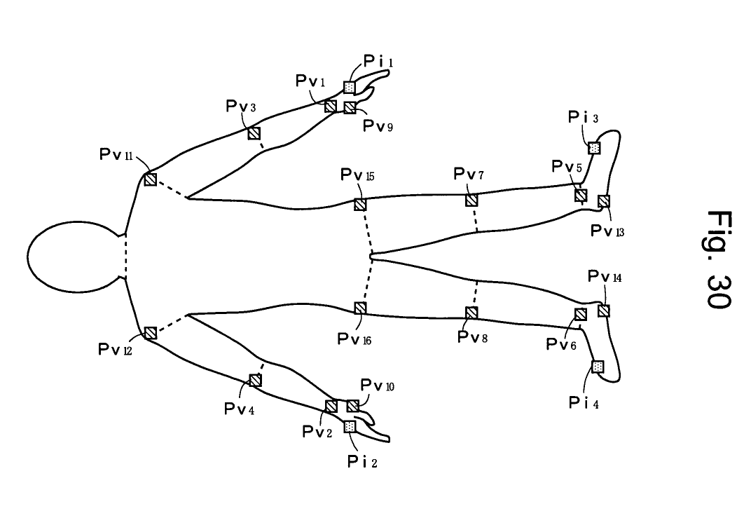

The characteristic measurement method performed in the data collection measurement is described below. In body composition measurement mode, there are four current-supplying points Pi₁-Pi₄ and four voltage-measuring points Pv₁-Pv₄ determined on the body of the subject. In data collection mode, on the other hand, the number of the voltage-measuring points is increased to sixteen to measure the impedance and estimate the body composition information more closely. FIG. 30 shows a figurative model of the human body indicating the electrode attachment positions for the data collection mode. The four current-supplying points Pi₁-Pi₄ are located in the vicinity of the root of the middle finger of the backs of the hands and the insteps of the feet. The current-supplying points Pi₁-Pi₄ may be located at any point as long as the points are on the distal side with respect to the measuring points (which will be described later) and adequately spaced from the measuring points. Fingers of both hands and both feet may be the current-supplying points, for example.

The voltage-measuring points Pv₁-Pv₁₆ correspond to four types of measurements: most distal, distal, proximal, and most proximal. Their positions are as follows.

| | |
|---|---|
| Most distal: | center of ulnar-side part of both palms, and heels of |
| | both feet (4 points) |
| Distal: | center of backs of both hands, and insteps of both |
| | feet (4 points) |
| Proximal: | radiale at both elbows, and outer tibiale at both |
| | knees (4 points) |
| Most proximal: | acromia of both shoulders, and greater trochanters of |
| | both legs (4 points) |

The distal and proximal measuring points Pv₁-Pv₈ are the same as in the measurement by the body composition measurement mode; the most distal and most proximal measuring points Pv₉-Pv₁₆ have been newly added.

As described above, the present body composition measurement apparatus has four measuring electrodes 11. In the body composition measurement mode, the measurement electrodes are rearranged once (distal→proximal), and the impedances of the limbs and the trunk are measured each time. Similarly, in the data collection mode, the measurement electrodes are rearranged three times (most distal→distal→proximal→most proximal), and the impedances of the limbs and the trunk are measured each time. There are fourteen types of measurements performable, as displayed in the target part display area C1 shown in FIG. 23. Each type of measurement has a different combination of two points for supplying the current and two points for measuring the voltage, as detailed below.

(1) Between arms: current is supplied between both hands; voltage is measured between both hands.

(2) Right arm: current is supplied between both hands; voltage is measured between right foot and right arm.

(3) Left arm: current is supplied between both hands; voltage is measured between left foot and left arm.

(4) Between legs: current is supplied between both feet; voltage is measured between both feet.

(5) Right leg: current is supplied between both feet; voltage is measured between right feet and right arm.

(6) Left leg: current is supplied between both feet; voltage is measured between left feet and left arm.

(7) Between right arm and right leg: current is supplied between right foot and right hand; voltage is measured between right foot and right arm.

(8) Trunk (with current supplied between right arm and right leg): current is supplied between right foot and right hand; voltage is measured between left foot and left arm.

(9) Between left arm and left leg: current is supplied between left foot and left hand; voltage is measured between left foot and left arm.

(10) Trunk (with current supplied between left arm and left leg): current is supplied between left foot and left hand; voltage is measured between right foot and right arm.

(11) Between right arm and left leg: current is supplied between right foot and left hand; voltage is measured between right foot and left arm.

(12) Trunk (between right arm and left leg): current is supplied between right foot and left hand;

voltage is measured between left foot and right arm.

(13) Between left arm and right leg: current is supplied between left foot and right hand; voltage is measured between left foot and right arm.

(14) Trunk (between left arm and right leg): current is supplied between left foot and right hand; voltage is measured between right foot and left arm.

Using the additional voltage-measuring points, the present measurement method makes it possible to measure the impedances of the additional four segments: right and left wrist areas, and right and left ankle areas (or heels), in addition to the aforementioned nine segments. In the case of repeatedly performing the measurement for every rearrangement of the four measuring electrodes 11, there are only four arrangements available for the measurement: most distal, distal, proximal and most proximal. Under such a condition, the voltage (or potential difference) of each segment can be measured as follows.

(1) When Current is Supplied Between Both Hands

The voltage ΔV₁ corresponding to the right and left wrist areas, the voltage ΔV₂ corresponding to the right and left forearms, and the voltage ΔV₃ corresponding to the right and left upper arms are given by:

ΔV₁=V₄−V₃,

ΔV₂=V₃−V₂,

ΔV₃=V₂−V₁,

where:

V₁ is the voltage between voltage-measuring points Pv₁₁ and Pv₁₂ located at the right and left acromia,

V₂ is the voltage between voltage-measuring points Pv₃ and Pv₄ located at at the right and left elbows,

V₃ is the voltage between voltage-measuring points Pv₁ and Pv₂ located at at the right and left wrists, and

V₄ is the voltage between voltage-measuring points Pv₉ and Pv₁₀ located at at the right and left palms.

In regard to the right side of the body, the voltage ΔVₐ corresponding to the right upper arm, the voltage ΔV_{b} corresponding to the right forearm, and the voltage ΔV_{c} corresponding to the right wrist area are given by:

ΔVₐ=V_{b}−Vₐ,

ΔV_{b}=V_{c}−V_{b},

ΔV_{c}=V_{d}−V_{c},

where:

Vₐ is the voltage between voltage-measuring points Pv₁₂ and Pv₁₆ located at right acromion and right greater trochanter,

V_{b} is the voltage between voltage-measuring points Pv₄ and Pv₈ located at right elbow and right knee,

V_{c} is the voltage between voltage-measuring points Pv₂ and Pv₆ located at right wrist and right ankle, and

V_{d} is the voltage between voltage-measuring points Pv₁₀ and Pv₁₄ located at right palm and right heel.

In regard to the left side of the body, the voltages corresponding to the upper arm, forearm and wrist area can be similarly calculated.

(2) When Current is Supplied between Both Feet

The voltage ΔV₁′ corresponding to the right and left ankle areas, the voltage ΔV₂′ corresponding to the right and left crura, and the voltage ΔV₃′ corresponding to the right and left thighs are given by:

ΔV₁′=V₄′−V₃′,

ΔV₂′=V₃′−V₂′,

ΔV₁′=V₂′−V₁′,

where:

V₁′ is the voltage between voltage-measuring points Pv₁₅ and Pv₁₆ located at the right and left greater trochanters,

V₂′ is the voltage between voltage-measuring points Pv₇ and Pv₈ located at the right and left knees,

V₃′ is the voltage between voltage-measuring points Pv₅ and Pv₆ located at the right and left ankles, and

V₄′ is the voltage between voltage-measuring points Pv₁₃ and Pv₁₄ located at the right and left heels.

In regard to the right side of the body, the voltage ΔVₐ′ corresponding to the right thigh, the voltage ΔV_{b}′ corresponding to the right crus, and the voltage ΔV_{c}′ corresponding to the right ankle area are given by:

ΔVₐ′=V_{b}−Vₐ,

ΔV_{b}′=V_{c}−V_{b},

ΔV_{c}′=V_{d}−V_{c},

where Vₐ, V_{b}, V_{c} and V_{d} are the voltages measured at the aforementioned points.

As described above, the data collection mode makes it possible to measure the impedances of the body of the subject more closely and accurately. Temporal change in impedance can be also measured. It is probable that an impedance changes with the heat beat, blood flow, respiration and other biorhythms of the human body. Therefore, it is possible to obtain information about the biorhythms of the human body by analyzing the temporal change of the impedance. This measurement has various applications. For example, it will be possible to give an external stimulus to the human body and measure the temporal change of an impedance. Thus, the measurement in the data collection mode is very useful to collect various kinds of information about human body. In the body composition measurement mode of the body composition measurement apparatus of the above embodiment, the body of the subject is subdivided into nine segments. This is because the subdivision of the arms into upper arms and forearms and the legs into thighs and crura not only improves the accuracy in the measurement of the body composition but also facilitates the application of the MRI method, as explained above. However, even in the case of dividing the body into five segments including the right and left arms each composed of upper arm and forearm, the right and left legs each composed of thigh and crus, and the trunk, the accuracy can be remarkably improved in comparison with conventional methods of estimating the body composition from the impedance between one hand and one foot by using an MRI method and creating the above-described multiple regression formulae.

In the above body composition measurement apparatus, the following measurement method can be adopted to improve the accuracy higher than obtained by the method using nine segments.

Impedances of the human body can be simply represented by an approximate model shown in FIG. 32. To improve the accuracy of the measurement, however, it will be preferable to use an approximate model that is closer to the real body. Among the impedances of all the segments, the segments relating to the limbs are modeled with considerable exactness. For the trunk, on the other hand, the modeling is not satisfactory because of the internal organs and other components contained in the trunk. Taking this into account, another model is created as shown in FIG. 37, where the trunk has a more detailed structure.

That is, the exactness of the model can be improved by assuming that there are impedances Z_{LTRH}, Z_{RTRH}, Z_{LTRL} and Z_{RTRL} between the impedance Z_{TRm} of the central part of the trunk and the roots of both arms (which is called the “internal part of shoulder” hereinafter) and the roots of the legs (which is called the “groin part” hereinafter”). This modeling provides higher exactness. The above impedances are not considered in the model shown in FIG. 32, which may cause a measurement error. Suppose such a case where the current is supplied between the backs of both hands and the voltage is measured between both wrists. In this case, the model shown in FIG. 32 does not include the impedance Z_{T} of the trunk, whereas the model shown in FIG. 37 includes the impedances Z_{LTRH}, Z_{RTRH} of the internal parts of both shoulders. This difference causes a measurement error.

[First Method of Estimating the Impedances of the Shoulder and the Groin Part]

One method of correcting the above influence from the impedance is to estimate the impedances of the internal part of the shoulder and the groin part from the impedances obtained by the above-described distal and proximal measurements. That is, first, the distal and proximal measurements are performed to measure the distal impedance Z₁ (between both wrists) and proximal impedance Z₂ (between both elbows) of the right side of the body.

Z₁=Z_{RFA}+Z_{RUA}+Z_{RTRH} (31)

Z₂=Z_{RUA}+Z_{RTRH} (32)

Therefore, the impedance of the right forearm, Z_{RFA}, is given by:

Z_{RFA}=Z₁−Z₂ (33).

Since forearm is highly correlated with upper arm,

Z_{RFA}∝Z_{RUA},

so that the following linear regression formula can be created:

Z_{RFA}=a₀×Z_{RUA}+b₀ (34).

where a₀ and b₀ are constants.

From formulae (33) and (34),

Z_{RFA}=Z₁−Z₂=a₀×Z_{RUA}+b₀

Z_{RUA}=(Z₁−Z₂−b₀)/a₀ (35).

Substituting formula (35) into formula (32),

Z₂=[(Z₁−Z₂−b₀)/a₀]+Z_{RTRH}

Z_{RTRH}=Z₂−[(Z₁−Z₂−b₀)/a₀].

With this formula, Z_{RTRH} can be calculated from Z₁ and Z₂. The impedance of the internal part of the left shoulder can be similarly calculated. Alternatively, it is possible to regard the impedance Z_{RTRH} of the internal part of the right shoulder as almost equal to the impedance Z_{LTRH} of the internal part of the left shoulder. Accordingly, using the above calculation result, the calculation may use the average value given as follows.

Z_{TRH}=(Z_{RTRH}+Z_{LTRH})/2

The impedances of the right and left groin parts can be similarly estimated.

[Second Method of Estimating the Impedances of the Shoulder and the Groin Part]

The impedance Z_{TRm} of the central part of the trunk has significant correlations with the impedances Z_{RTRH}, Z_{LTRH} of the internal parts of the shoulders and the impedances Z_{RTRL}, Z_{LTRL} of the groin part. Taking into account such correlations, the impedances can be given as follows:

Z_{RTRH}=f₁(Z_{TRm}),

Z_{LTRH}=f₂(Z_{TRm}),

Z_{RTRL}=f₃(Z_{TRm}),

Z_{LTRL}=f₄(Z_{TRm}),

where f₁, f₂, f₃ and f₄ are correlation functions. In addition, body specific information, such as height (H), weight (W), age (Ag) and gender (Sx), may be introduced as follows.

Z_{RTRH}=f₁(Z_{TRm}, W, Ag, Sx)

Z_{LTRH}=f₂(Z_{TRm}, W, Ag, Sx)

Z_{RTRL}=f₃(Z_{TRm}, W, Ag, Sx)

Z_{LTRL}=f₄(Z_{TRm}, W, Ag, Sx)

Furthermore, it is possible that the impedances Z_{RTRH}, Z_{LTRH} of the internal parts of the shoulders are correlated with the impedances Z_{RA}, Z_{LA} of the arms and the impedances Z_{RTRL}, Z_{LTRL} of the groin parts are correlated with the impedances Z_{RL}, Z_{LL} of the legs. These correlations may be taken into account as follows,

Z_{RTRH}=f₁′(Z_{TRm}, Z_{RA}),

Z_{LTRH}=f₂′(Z_{TRm}, Z_{LA}),

Z_{RTRL}=f₃′(Z_{TRm}, Z_{RL}),

Z_{LTRL}=f₄′(Z_{TRm}, Z_{LL}),

or

Z_{RTRh}=f₁′(Z_{TRm}, Z_{RA}, W, Ag, Sx),

Z_{LTRh}=f₂′(Z_{TRm}, Z_{LA}, W, Ag, Sx),

Z_{RTRL}=f₃′(Z_{TRm}, Z_{RL}, W, Ag, Sx),

Z_{LTRL}=f₄′(Z_{TRm}, Z_{LL}, W, Ag, Sx).

In the above estimation formulae using the correlations, it is possible to eliminate the impedance Z_{TRm} of the central part of the trunk and use only the impedances Z_{RA}, Z_{LA} of the arms or the impedances Z_{RL}, Z_{LL} of the legs. That is:

Z_{RTRh}=f₁″(Z_{RA}),

Z_{LTRh}=f₂″(Z_{LA}),

Z_{RTRL}=f₃″(Z_{RL}),

Z_{LTRL}=f₄″(Z_{LL}),

or

Z_{RTRh}=f₁″(Z_{RA}, W, Ag, Sx),

Z_{LTRh}=f₂″(Z_{LA}, W, Ag, Sx),

Z_{RTRL}=f₃″(Z_{RL}, W, Ag, Sx),

Z_{LTRL}=f₄″(Z_{LL}, W, Ag, Sx).

The impedances of the arms and legs used here are the impedances of the limbs measured by the most distal, distal, proximal or most proximal measurement.

Thus, it is possible to increase the accuracy of the impedances of the segments by estimating and using the impedances of the internal parts of the shoulders and the groin, as described above. From the impedances thus obtained, the body composition information can be estimated with higher accuracy.

As described above, the body composition measurement apparatus of the first embodiment provides various kinds of body composition information with high accuracy by a method that facilitates the work and operation to be done by the examiner and barely imposes physical or mental burdens on the subject. By setting the focus of the measurement not only on body-fat but also on muscle and/or bone and displaying indices relating to the muscle and/or bone, it is possible to provide information useful for training of athletes or health management of elderly people, or other information that cannot be easily provided by conventional apparatuses.

The apparatus of the first embodiment uses affix-type electrodes as the current-carrying electrodes 10. Alternatively, it is possible to use clip-type electrodes designed to clip a finger of each hand instead of the back of the hand, or a finger of each foot instead of the instep of the foot, to ensure conductive connections. Clip-type electrode can be repeatedly used, so that the running cost becomes less than in the case of using disposable affix-type electrodes. Affix-type electrodes easily come off the body when pulled by the cable, causing a contact failure. Clip-type electrodes, in contrast, hardly cause such a contact failure, and are easy to handle. It should be noted that, when a finger is chosen as a current-supplying point, the current source 203 must have a considerably high driving power, because the impedance of the finger is added to the current path. The closer the point is to the fingertip, the higher the power must be.

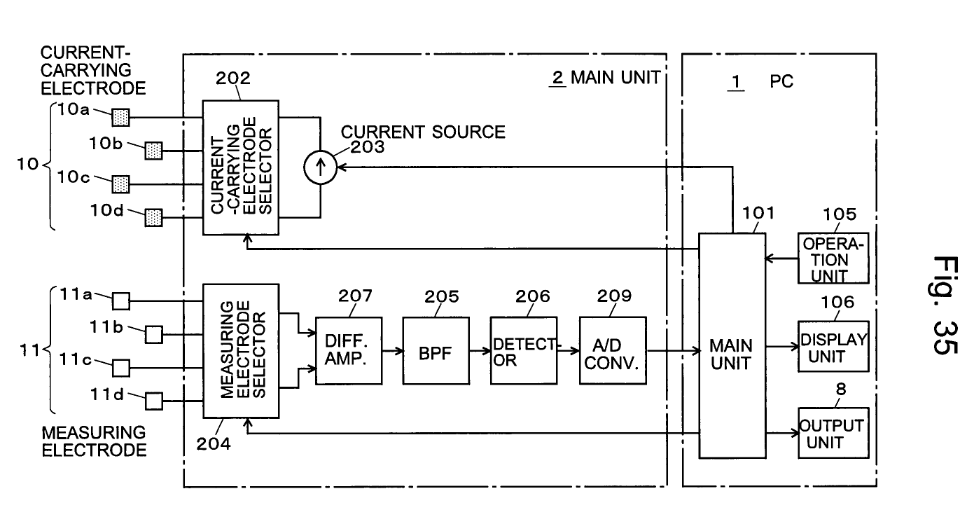

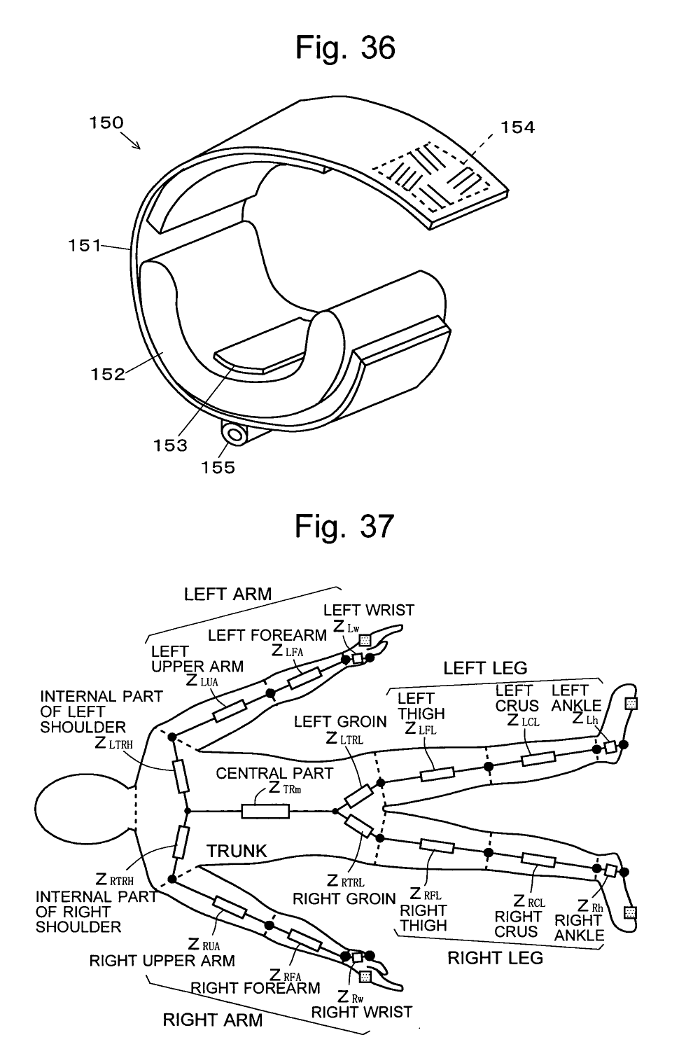

When a finger is chosen as a current-supplying point, a thimble-type electrode unit 150 as shown in FIG. 36 may be used. The electrode unit 150 includes an outer band 151 made of a cloth or similar material with an elastic member 152 attached inside, and an electrode 153 attached to the inside of the elastic member 152. The electrode 153 is conductively connected with a socket 155, into which the cable 4 can be plugged. When the unit 150 is fastened around a finger with the velcro (TM) 154, the electrode 153 comes in stable contact with the cushion or other part of the finger.

The body composition measurement apparatus of the first embodiment is composed of a multi-purpose notebook computer and a main unit enclosing electrical circuits not included in the computer. It should be taken for granted that the apparatus may be constructed in other forms. For example, a desktop computer may be used instead of the notebook computer. This construction makes it possible to produce an expansion board that is equivalent to the above main unit in function, and to install the board in the expansion unit of the PC. It is of course possible to use other interface as the interface for connecting the PC and the main unit. It is also possible to enclose the entire function units in a casing without using a multi-purpose personal computer.

It should be also taken for granted that the body composition measurement apparatus according to the present invention may be constructed to include only a part of the components of the body composition measurement apparatus described in the first embodiment, and embody a part of its functions. For example, the apparatus of the first embodiment is constructed to estimate ADL indices based on the measurement values of the impedances of the body of the subject, and display the ADL indices on the screen. As explained above, the ADL indices estimated here is very useful for elderly people or trainees of rehabilitation. Taking this into account, it is possible to construct a simplified body composition measurement apparatus that calculates and displays only ADL indices, or only ADL indices and limited items of body composition information. The ADL indices used here relate only to the quadriceps. Therefore, all that is necessary is to measure the impedances of the thigh or crus; there is no need to measure the impedances of the upper limbs. It is preferable to independently measure the impedances of the thighs or crura on both sides of the body and estimate the muscle masses of the right and left quadricepses from the impedances of the thighs or crura and the body specific information. As a simplified case, however, it is allowable to measure the impedance between the ankles through the legs and estimate the total muscle mass of the right and left quadricepses from the impedance. Once the muscle mass of the quadriceps is known, it is possible to estimate the maximum muscle force of the quadriceps and the weight-bearing index.

When, as described above, only the impedance of the lower limb is to be measured, the apparatus can be constructed with a small number of current-carrying electrodes and measuring electrodes having simplified structures. For example, similar to conventional body-fat meters, the apparatus may have a platform provided with electrodes located to contact the feet when the subject steps on it. In this case, some decrease in accuracy is expected because the voltage-measuring path includes the ankles. Therefore, the apparatus should be preferably constructed to measure the voltage between both ankles or both knees. It is possible to define a different ADL index relating to a muscle of some part of the body other than the leg, such as a hand or back. For example, there may be an ADL index indicative of the power to grasp an object with a hand or the power to lift up an object. In such a case, the apparatus should be constructed to measure the impedance of a body part from which the muscle mass of the part concerned can be estimated.

As another embodiment of the present invention, the body composition measurement apparatus described below includes the above-described modifications.

[Second Embodiment]

The second embodiment of the body composition measurement apparatus according to the present invention is described. The body composition measurement apparatus of the second embodiment focuses on the wrist area or the ankle area as a part of the human body where the ratio of the mass of the osseous tissue is particularly high. In this embodiment, dedicated measuring electrodes are attached to the aforementioned body parts to measure the impedance that strongly reflects the mass of the osseous tissue, and the mass of the osseous tissue is estimated from the measurement value of the impedance and the body specific information.

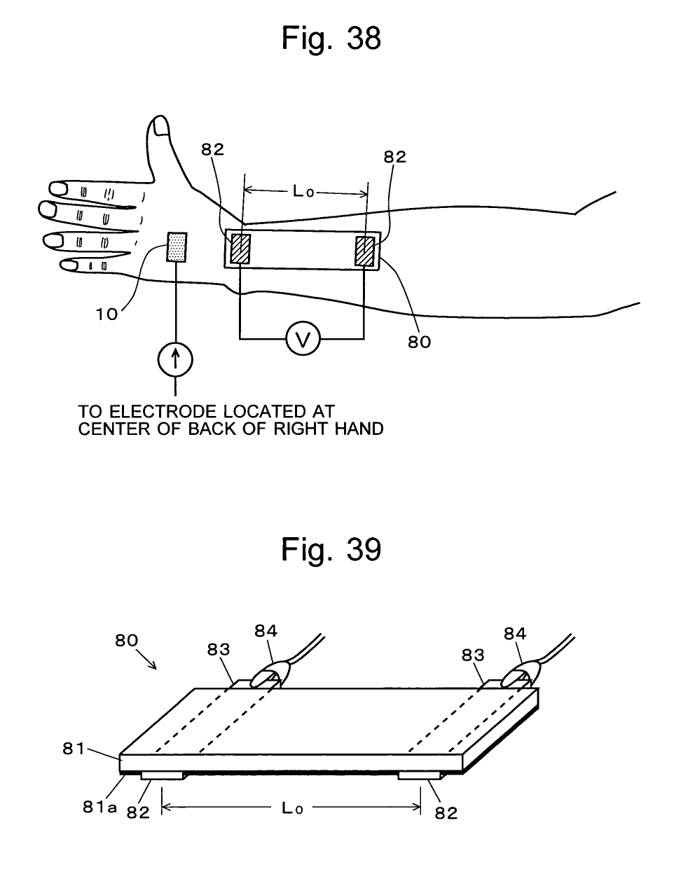

FIG. 38 shows an electrode pad 80 attached to the arm in the vicinity of the wrist, and FIG. 39 is an external perspective view showing the electrode pad 80. In FIG. 39, the base tape 81 is a thin sheet made of an insulating material, such as polyethylene or polyvinyl chloride. The base tape 81 has a pair of strip-shaped electrodes 82 made of a conductive gel, spaced apart by a predetermined distance L₀. On the face of the base tape 81 where the electrodes 82 are formed, an insulating adhesive layer 81a is formed at the area other than the electrodes 82, providing adequate adhesion to the skin of the subject. At one side of the base tape 81, a pair of terminals 83 leading to the electrodes 82 are pulled out. The aforementioned cable 4 can be connected to the terminals 83 by pinching the terminals 83 with clip-like connectors 84.

In the measurement, the electrode pad 80 is affixed to an arm of the subject on the side of the back of the hand, from the wrist joint to an upper position. The electrode 82 closer to the wrist coincides with the voltage-measuring point Pv₁ or Pv₂ located at the wrist. Accordingly, with the electrode pad 80 attached to one or both wrists, the electrode 82 located closer to the wrist can be used as the measuring electrode for the distal measurement in the first embodiment. As an example of the current-carrying electrode 10, an electrode attached to the back of the hand, as described in the first embodiment, can be used as is. By using this electrode pad 80 with the affix-type electrodes described in the first embodiment, it possible to additionally measure only the wrist part after normally carrying out the distal and proximal measurements.

In the vicinity of the wrist, the subcutaneous fat and muscle tissue are thin, and the percentage of the osseous tissue is a larger than that of the muscle or fat. That is, in the model shown in FIG. 33A, the osseous tissue occupies a large space in cross-sectional area. Accordingly, when, for example, the voltage is measured between the electrodes 82 with a radio frequency current supplied between both hands and the impedance is derived from the current value and the voltage value, the impedance contains a good deal of information about the osseous tissue. Therefore, using this measurement value of the impedance, it is possible to not only accurately calculate the bone mass of the body part concerned, but also improve the accuracy of estimating the bone mass of the entire body. Furthermore, the detailed information about the osseous tissue will be useful in the examination of the bone density or the progress of the osteoporosis.

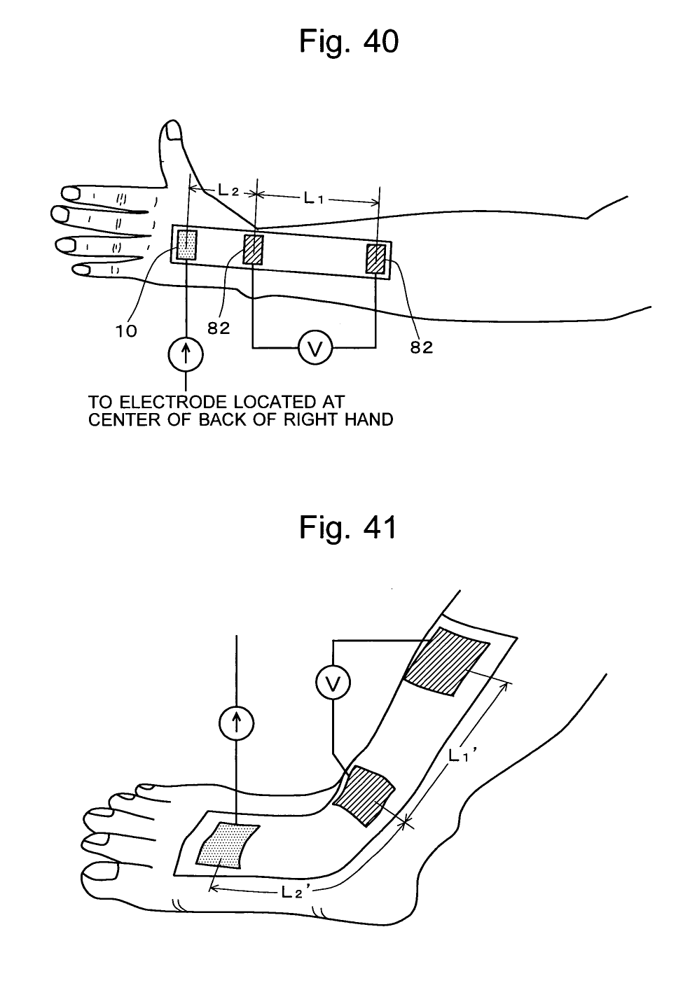

FIG. 40 shows another example of the electrode pad having a different form, attached to the wrist. As in this example, the current-carrying electrode 10 may be also fixed to the base tape 81. FIG. 41 shows another electrode pad formed similar to the above electrode pad attached to the ankle. The part of the body extending from the joint at the ankle to an upper part (i.e. shin) has also a large proportion of osseous tissue in cross-sectional area. Therefore, it is possible to perform the above measurement with an electrode pad attached in the vicinity of the ankle.

In the measurement using the body composition measurement apparatus of the first embodiment, it is assumed that the subject takes a supine position, as shown in FIG. 31. Different positions might be allowable, but it would normally decrease the accuracy of the measurement. Compared with conventional apparatuses in this field, the present apparatus makes the measurement higher inaccuracy and simpler in operation. However, the apparatus still has problems that the electrodes must be attached to the body of the subject, and that it is difficult for the subject to do the measurement alone. Depending on the purpose of use, the user may desire to do the measurement in a simpler and easier way, even if it somewhat decreases the accuracy. In view of the above matters, the following embodiment is designed to make the measurement performable in a simpler way.

[Third Embodiment]

FIG. 42 shows the state of use of a body composition measurement apparatus as the third embodiment. The body composition measurement apparatus 40 has an upper limb measuring unit 41 to be held by the subject with both hands and a lower limb measuring unit 42 which the subject steps on. These units are connected with each other via a cable 43. The functions corresponding to the PC 1 and the main unit 2 of the first embodiment are built in the upper limb measuring unit 41. FIG. 45 is an external perspective view of the upper limb measuring unit 41. The upper limb measuring unit 41 has a U-shaped body 411 with both ends directed backwards. The body 411 has columnar grips 412L and 412R at both ends directed backward. Each grip 412L or 412R has a current-carrying electrode 413L or 413R and a measuring electrode 415L or 415R in the upper and lower parts of its circumferential wall, respectively, with a gap between them. The body 411 also has additional measuring electrodes 414L and 414R on the outer sides of the bending parts. On the front side of the central part of the body 411, a display unit 416 composed of a liquid crystal panel for displaying characters, digits, figures, etc., is located between the measuring electrodes 414L and 414R. In addition, the body 411 is provided with operation switches (not shown in the drawing).

In the measurement, the subject holds the right and left grips 412L and 412R with both hands, with both thumbs put on the front sides of the upper parts of the grips 412L and 412R and with the other fingers put on their back sides, and stretches out both arms substantially forwards. Then, the entire part of the thumbs and the cushions of the index and middle fingers contact the current-carrying electrodes 413L and 413R, both palms contact the right and left measuring electrodes 415L and 415R, and the insides of both wrists contact the right and left measuring electrodes 414L and 414R. Thus, the current-supplying points Pi₁, Pi₂ and the voltage-measuring points Pv₁, Pv₂, Pv₉ and Pv₁₀ shown in FIG. 32 are determined. The current-carrying electrode 413L (and 413R) and the measuring electrode 415L (and 415R) may exchange their functionalities without causing essential change in performance.

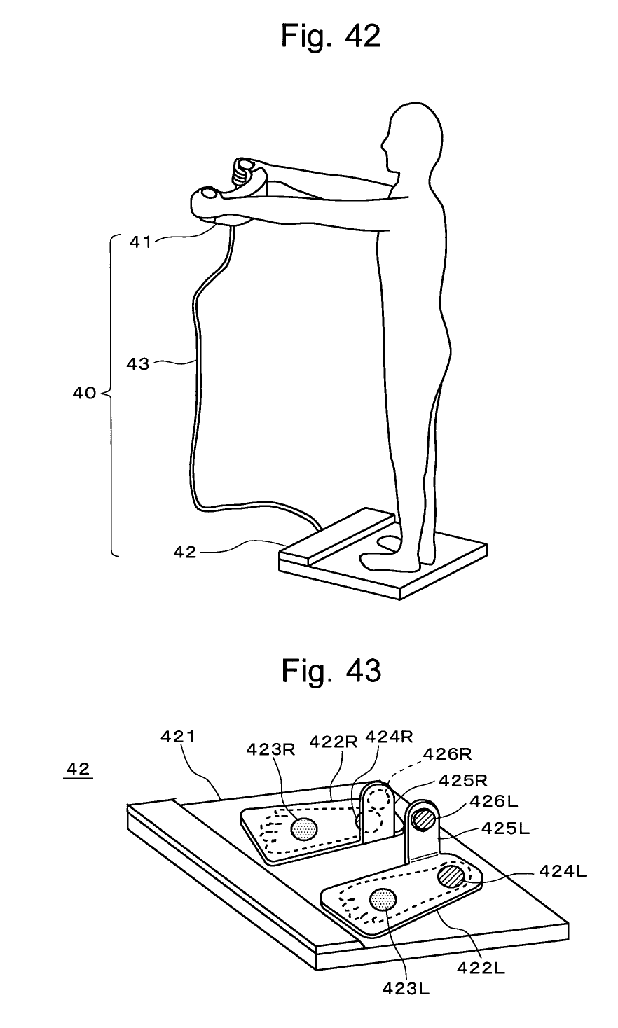

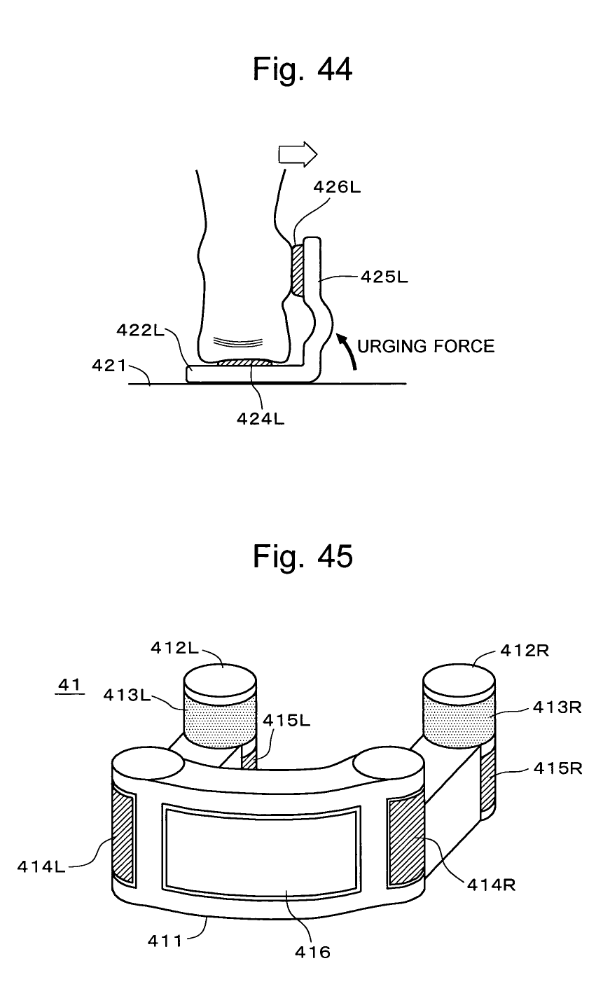

FIG. 43 is an external perspective view of the lower limb measuring unit 42, and FIG. 44 is an enlarged view of the state of measurement using the lower limb measuring unit 42. The lower limb measuring unit 42 has right andleft foot-positioning parts 422L and 422R located on a plate-like measuring platform 421. The foot-positioning parts 422L and 422R, each being one size larger than the outline of a normal-size foot, are provided with a current-carrying electrode 423L or 423R located in its front side (i.e. finger-side) area and a measuring electrode 424L or 424R located in its rear side (i.e. heel-side) area. Spring-like standing plates 425L and 425R, standing almost upright, are located at the insides of the heels of the foot-positioning parts 422L and 422R, respectively. Each standing plate 425L or 425R has a measuring electrode 426L or 426R in the upper part of the outside face. In the measurement, when the subject steps on the foot-positioning parts 422L and 422R, the current-carrying electrodes 423L and 423R contact the finger-side parts of the soles, and the measuring electrodes contact the heel-side parts of the soles. Next the subject closes the legs inwards. Then, as shown in FIG. 44, the measuring electrode 426L contacts the inside of the ankle of the subject, because the standing plates 425L and 425R are urged outwards. It should be noted that FIG. 44 shows only the left foot side, and the right foot side becomes its mirror image. Thus, the current-supplying points Pi₃ and Pi₄ and the voltage-measuring points Pv₅ and Pv₆ shown in FIG. 32 are determined. In addition, voltage-measuring points Pv₁₃ and Pv₁₄ for measuring the impedances Z_{Lh} and Z_{Rh} of the right and left ankles are determined at the heels on the soles. As in the case of the hands, the current-carrying electrode 423L (and 423R) and the measuring electrode 424L (and 424R) may exchange their functionalities without causing essential change in performance.

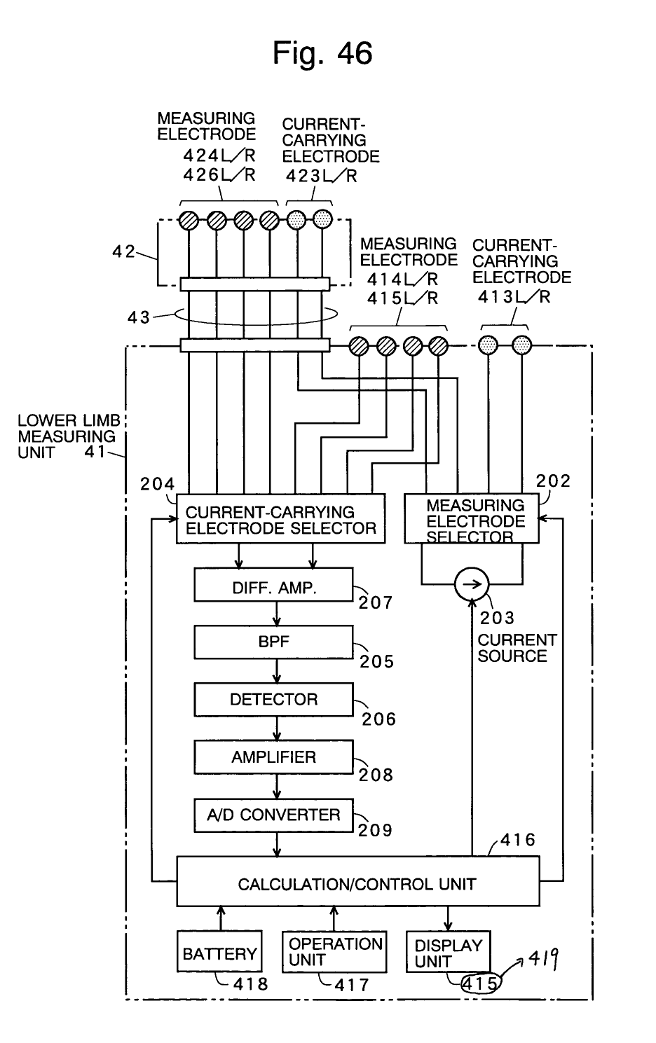

FIG. 46 shows the electrical construction of the body composition measurement apparatus of the third embodiment. The basic construction is the same as that of the body composition measurement apparatus of the first embodiment. Accordingly, the same numerals are given to the same or corresponding parts, and the description is omitted here. The present apparatus has two current-carrying electrodes 423L and 423R to contact the soles at the roots of the fingers and four measuring electrodes 424L, 424R, 426L and 426R to contact the heels of the soles and the insides of the ankles. These electrodes are connected via a cable 43 to the current-carrying electrode selector 202 and the measuring electrode selector 204 in the upper limb measuring unit 41. The limb measuring unit 41 has two current-carrying electrodes 413L and 413R to contact the fingers of both hands and four measuring electrodes 415L, 415R, 414L and 414R to contact both palms and the insides of both wrists. These electrodes are connected via internal wiring to the current-carrying electrode selector 202 and the measuring electrode selector 204. The calculation/control unit 416 substitutes for the PC main unit 101 and CPU 211 of the apparatus in the first embodiment.

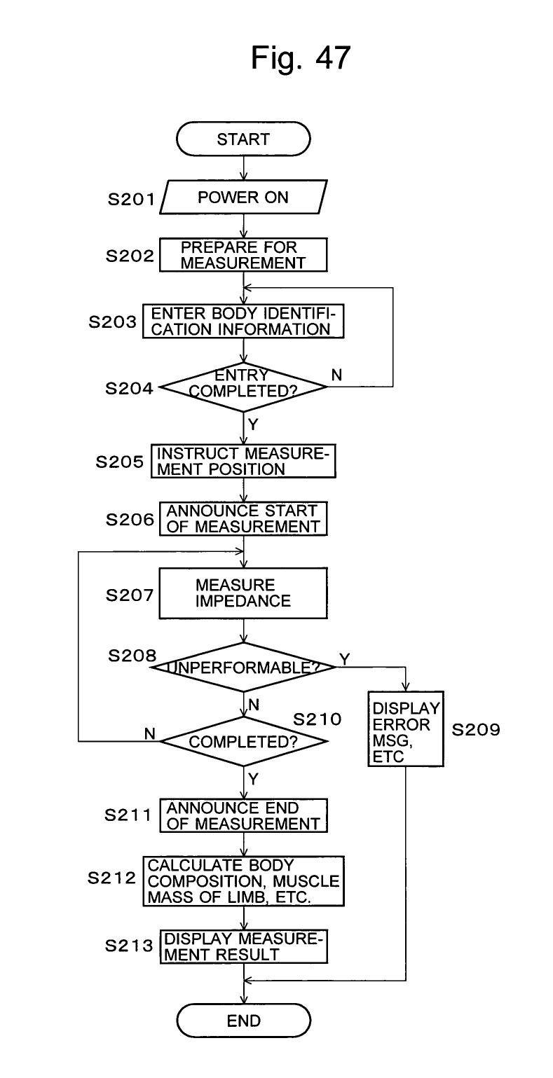

The process of the measurement using the present apparatus is explained along the flowchart of FIG. 47. The subject presses the power switch of the upper limb measuring unit 41 to turn the power ON (Step S201). Then, the apparatus starts running and performs the measurement preparation process including the initialization, self-checking of measurement circuits, etc (Step S202). Next, the subject enters the body specific information, such as the height, weight, age and gender, by operating the switches of the operation unit 417 or other parts (Step S203). After that, it is determined whether all items of information have been entered (Step S204). When any item of information is missing, the operation returns to Step S203. When, in Step S204, it is determined that all items of information have been entered, an instruction for urging the subject to take the measurement position is displayed on the display unit or given by voice (Step S205). According to the instruction, the subject takes a standing position with the feet placed on the foot-positioning parts 422L and 422R, holds the grips 412L and 412R of the upper limb measuring unit 41 with both hands, as described above, and stretches out the arms and keep them at shoulder height. The legs should be intentionally closed slightly inwards so that the measuring electrodes 426L and 426R contact the insides of the ankles. The above-described positioning brings the fingers of both hands and the finger-side parts of both soles into contact with the current-carrying electrodes 413L, 413R, 423L and 423R, respectively. Also, both palms, the insides of both wrists, the heel-side parts of both soles and the insides of both ankles are brought into contact with the measuring electrodes 415L, 415R, 414L, 414R, 424L, 424R, 426L and 426R, respectively.

Next, start of the measurement is announced on the display unit 416 (Step S206), and the measurement of the impedance is started. That is, the current-carrying electrodes 10 and the measuring electrodes 11 are switched by the current-carrying electrode selector 202 and the measuring electrode selector 204 so that the right arm, left arm, right leg, left leg and trunk are sequentially selected as the target part. A weak radio frequency current is supplied between two current-carrying electrodes 10 selected, and the electrical potential generated by the current is measured one after another with two measuring electrodes 11. The process of measuring the impedance from Step S207 to Step S210 is similar to the distal measurement described in the first embodiment, so that the description is omitted here. It should be noted, however, that it is possible in the present (third) embodiment to additionally measure the impedances of the right and left wrist areas with the voltage-measuring points located at the right and left palms, and to additionally measure the impedances of the right and left ankle areas with the voltage-measuring points located at the ankle-side part of the right and left soles. After the measurement is completed, the completion is announced by, for example, a measurement completion message on the display unit 416 (Step S211). After this announcement, the subject is allowed to be freed from the measurement position. After that, a predetermined calculation is performed using the measurement values of the impedances and the body specific information to obtain body composition information and/or health condition check information (Step S212), and the result is displayed on the display unit 415 (Step S213).

As described above, the body composition measurement apparatus of the third embodiment allows the subject to take a standing position and perform the measurement without taking a supine position. Furthermore, the subject can perform the measurement alone. Accordingly, the subject can feel free to do the measurement without feeling much reluctance. The externals and construction of the body composition measurement apparatus of the third embodiment may be modified in various ways. For example, the electrical circuits may be enclosed not in the upper limb measuring unit 41 but in the lower limb measuring unit 42. The upper limb measuring unit 41 and the lower limb measuring unit 42 may be constructed as independent apparatuses. The apparatus may be modified to perform the measurement in which one hand and one foot are paired.

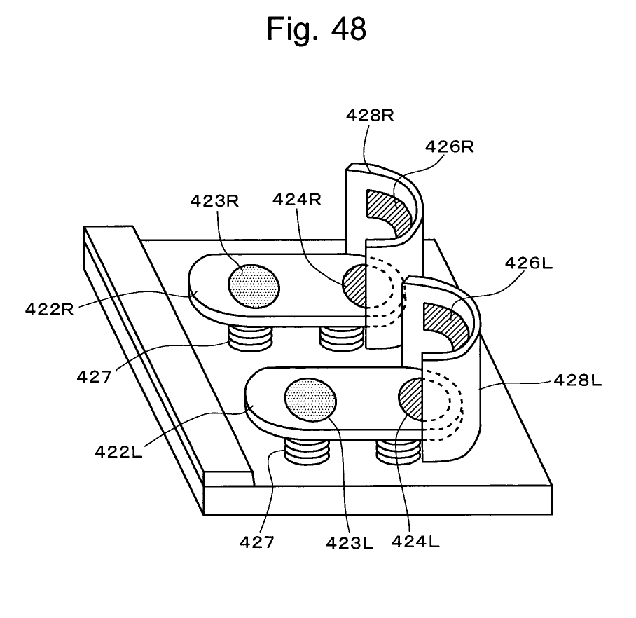

FIG. 48 is an external view showing a modified example of the lower limb measuring unit 42. In this example, the unit has foot-positioning parts 422L and 422R urged upwards by springs 427, semicylindrical walls 428L and 428R standing to cover the backs of the ankles, and measuring electrodes 426L and 426R fixed to the upper parts of the insides of the semicylindrical walls. This structure uses the urging force of the springs 427 to strengthen the contact of the current-carrying electrodes 423L and 423R and the measuring electrodes 424L and 424R with the soles when the subject steps on the foot-positioning parts 422L and 422R.

[Fourth Embodiment]

In the measurement using the body composition measurement apparatus of the third embodiment, the subject must take the standing position with the arms kept apart from the trunk (preferably, the arms should be maintained stretched out). This positioning may be difficult for elderly people or those who are subjected to medical treatments. Furthermore, the apparatus does not perform the proximal measurement using the elbows and knees as voltage-measuring points, so that the accuracy of estimating the body composition information is slightly low. The body composition measurement apparatus of the fourth embodiment is improved in this respect.

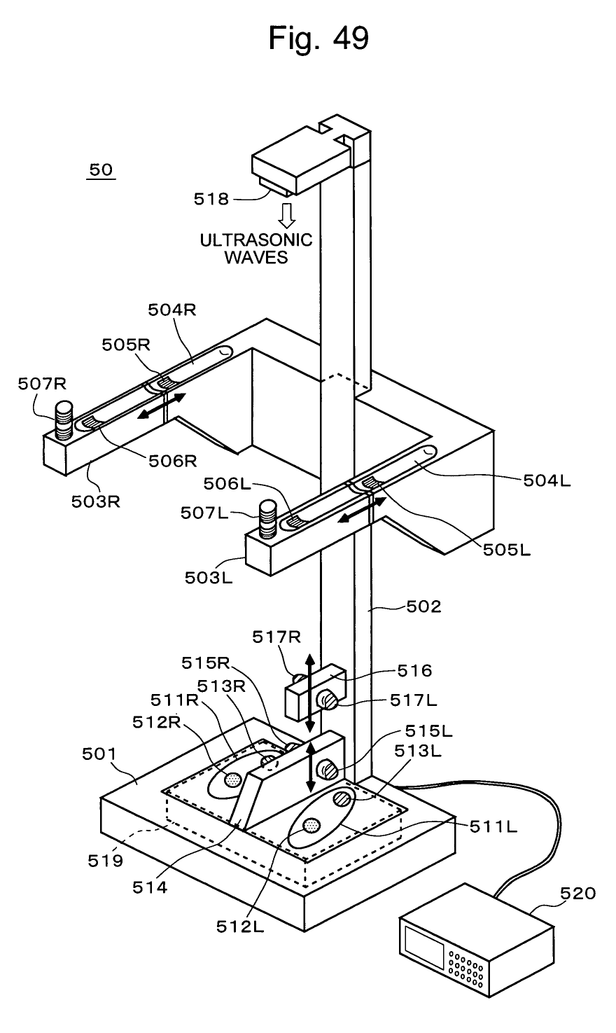

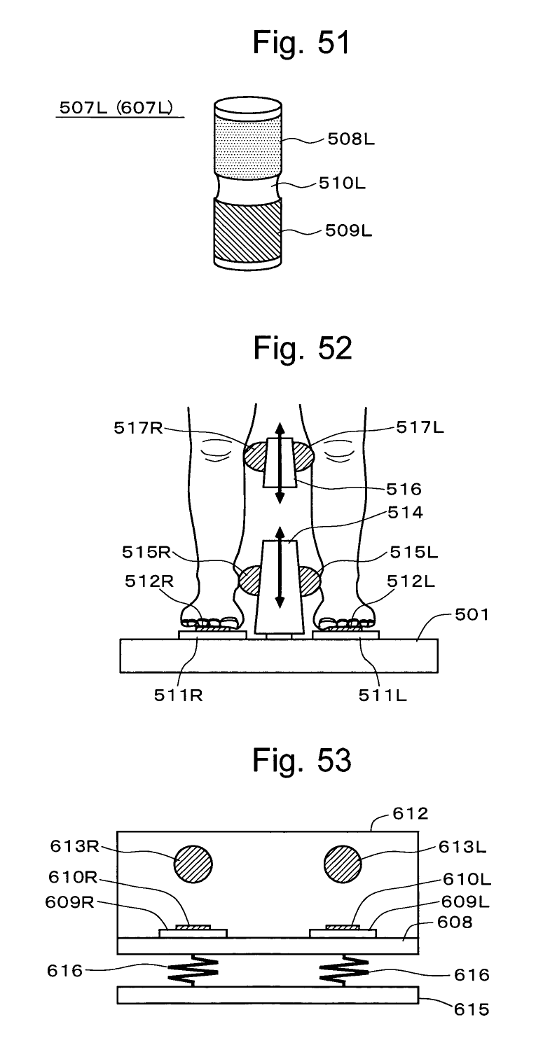

FIG. 49 is an external view of a body composition measurement apparatus 50 as the fourth embodiment. The apparatus 50 has a support post 502 standing on a measuring platform 501 and armrests 503L and 504R for measuring the upper limbs, where the armrests 503L and 504R are vertically movable. The armrests 503L and 504R have recesses 504L and 504R on their top, which determine the positions to rest the arms. The recesses 504L and 504R have measuring electrodes 505L and 505R to contact the arms in the vicinity of the elbows, and measuring electrodes 506L and 506R to contact the arms in the vicinity of the wrists. The armrests 503L and 503R are designed expansible and contractible so that the distance between the measuring electrodes 505L and 506L, or 505R and 506R, can be adjusted to the length of the arm. The armrests 503L and 503R also have grips 507L and 507R on the top of the ends, which are to be held with both hands. The grip 507L has a columnar body, as shown in FIG. 51, having a current-carrying electrode 508L in its upper part and a measuring electrode 509L in its lower part, with a groove-like insulating zone 510L separating the two electrodes. The grip 507R on the right side is similarly constructed. When the grip 507L is held with a hand, with its middle finger put over the insulating zone 510L, then the cushions of the index finger and the thumb contact the current-carrying electrode 508L, and a range including the third finger, little finger and ulnar-side part of the palm, contacts the measuring electrode 509L. The measuring platform 501 has foot-positioning parts 511L and 511R, similar to the body composition measurement apparatus of the third embodiment. Each foot-positioning part 511L or 511R has a current-carrying electrode 512L or 512R in the finger-side area and a measuring electrode 513L or 513R in the heel-side area. Between the right and left foot-positioning parts 511L and 511R, an ankle-measuring projection 514 stands, with measuring electrodes 515L and 515R on both sides, which are to contact the insides of both ankles. A vertically movable knee-measuring projection 516, projecting forwards from the support post 520, has measuring electrodes 517L and 517R on both sides, which are to contact the insides of both knees.

An ultrasonic range finder is mounted on the top of the support post 502, directed downwards, whereby the height of the subject standing in front of the support post 502 can be measured. The measuring platform 501 contains a built-in weighing machine 519. The height and weight are automatically measured and used as the body specific information. In the present apparatus, the electrical circuits enclosed in the upper limb measuring unit 41 of the third embodiment are enclosed in a circuit unit 520, which is separated from the measuring unit having the aforementioned electrodes. Both units are connected with each other via a cable. The electric circuits are constructed similar to those used in the third embodiment. Accordingly, the description is omitted here.

As shown in FIG. 52, the subject stands on the measuring platform 501 with both feet placed on the foot-positioning parts 511L and 511R, and closes the legs inwards to bring the insides of the right and left ankles into contact with the measuring electrodes 515L and 515R, and the insides of the right and left knees into contact with the measuring electrodes 517L and 517R. The armrests 503L and 503R are vertically moved to a position where both arms can rest on them without difficulty, and further expanded or contracted so that the subject can hold the grips 507L and 507R with the arms rested in the recesses 504L and 504R. This positioning brings the cushions of the thumbs and the index fingers of both hands and the finger-side parts of the soles into contact with the current-carrying electrodes 508L, 508R, 512L and 512R, respectively, whereby the current-supplying points Pi₁, Pi₂, Pi₃ and Pi₄ shown in FIG. 32 are determined. Furthermore, the ulnar-side parts of both palms, both wrists, both elbows, heel-side part of the soles of both feet, insides of both ankles and insides of both knees contact the measuring electrodes 509L, 509R, 506L, 506R, 505L, 505R, 513L, 513R, 515L, 515R, 517L and 517R, respectively, whereby the voltage-measuring points Pv₁-Pv₈ shown in FIG. 32 are determined and, furthermore, the measuring points for measuring the impedances Z_{Lh} and Z_{Rh} of the right and left ankle and the impedances Z_{Lw} and Z_{Rw} of the right and left wrists are determined.

In the body composition measurement apparatus of the fourth embodiment, the voltage-measuring points are located also at the elbows and the knees. Therefore, similar to the body composition measurement apparatus of the first embodiment, it is possible to separately perform the distal measurement and the proximal measurement, and to measure the wrist area and the ankle area each as a single body part. Thus, while allowing the standing position, the apparatus can perform the measurement with higher accuracy than the body composition measurement apparatus of the third embodiment. Its automatic measurement of height and weight eliminates the troublesome work of entering these data as body specific information. Furthermore, the support of the arms with the armrests 503L and 503R not only reduces the fatigue of the arms, but also increases the accuracy of the measurement because the arms are prevented from moving up and down during the measurement.

[Fifth Embodiment]

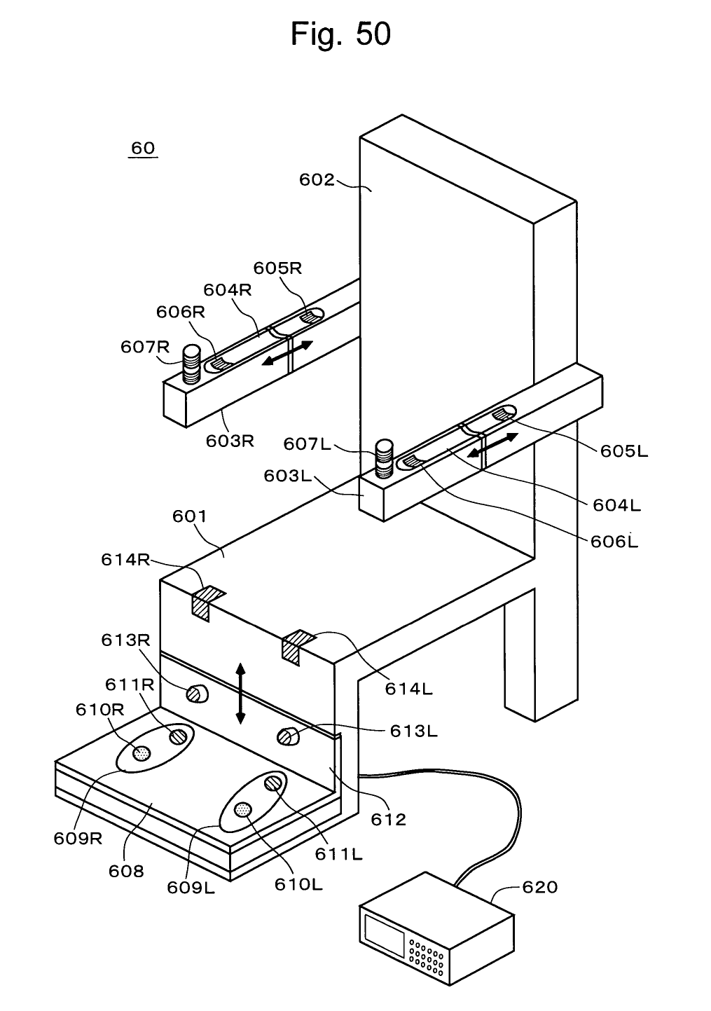

For those who have difficulties in taking the standing position, it will be convenient that the measurement can be performed with a sitting position. FIG. 50 is an external view of a body composition measurement apparatus 60 as the fifth embodiment. The body composition measurement apparatus is constructed like a chair with the armrests 603L and 603R on both sides of the backrest 602. The armrests 603L and 603R are constructed similar to the armrests 503L and 503R of the body composition measurement apparatus of the fourth embodiment, but are different in that the recesses 604L and 604R are designed to support only the elbows and the forearms. The recesses 604L and 604R have measuring electrodes 605L and 605R to contact the arms in the vicinity of the elbows, and measuring electrodes 606L and 606R to contact the arms in the vicinity of the wrists. The grips 607L and 607R are constructed similar to the grips of the body composition measurement apparatus of the fourth embodiment, shown in FIG. 51. The seat 601 has measuring electrodes 614L and 614R located at the front-side corners, which contact the backs of the knees when the subject sits down on the seat 601. A measuring platform 608 with foot-positioning parts 609L and 609R is located so that both feet can be placed on it. Similar to the apparatuses of the third and fourth embodiments, each foot-positioning part 609L or 609R has a current-carrying electrodes 610L or 610R located on the finger-side and a measuring electrode 611L or 611R located on the heel side. A vertical front pedestal plate 612 is integrally connected to the measuring platform 608. The front pedestal plate 612 has measuring electrodes 613L and 613R extending forwards from the front face, which are to contact the backs of the ankles.

FIG. 53 is a front view of the measuring platform 608 and its surroundings. The measuring platform 608 is urged upwards by springs 616 against a base 615 placed on the floor. When the subject sits down on the seat 601 with the feet placed on the foot-positioning parts 609L and 609R, the measuring platform 608 sinks to some extent according to the height of the knees from the soles. Therefore, the current-carrying electrodes 610L and 610R and the measuring electrodes 611L and 611R assuredly contact the soles, and the measuring electrodes 614L and 614R contact the backs of the knees. Next, the subject deeply sits down on the seat 601 and straightens the spine, leaning against the backrest 602. The armrests 603L and 603R are vertically moved to a position where both arms can be easily rested, and further expanded or contracted so that the subject can hold the grips 607L and 607R with the arms rested in the recesses 604L and 604R. Then, the underarms are made slightly open so that the arms are prevented from touching the trunk. This positioning brings the cushions of the thumbs and the index fingers of both hands and the finger-side parts of both soles into contact with the current-carrying electrodes 508L, 508R, 610L and 610R, respectively, whereby the current-supplying points Pi₁, Pi₂, Pi₃ and Pi₄ shown in FIG. 32 are determined. Furthermore, the ulnar-side parts of both palms, both wrists, both elbows, heel-side part of both soles, insides of both ankles and insides of both knees contact the measuring electrodes 509L, 509R, 606L, 606R, 605L, 605R, 611L, 611R, 613L, 613R, 614L and 614R, respectively, whereby the voltage-measuring points Pv₁-Pv₈ shown in FIG. 32 are determined and, furthermore, the measuring points for measuring the impedances Z_{Lh} and Z_{Rh} of the right and left ankle and the impedances Z_{Lw} and Z_{Rw} of the right and left wrists are determined. Thus, the voltage-measuring points are determined on the body of the subject at the same positions as in the case of the apparatus of the fourth embodiment, so that the measurement can be performed in the same way. This construction allows the subject to take a sitting position during the measurement similar to the fourth embodiment, so that the physical burden on the subject is reduced. In this mode, the chair may be constructed as a reclining sheet type.

It should be noted that the above embodiments are mere examples of the present invention. They can be variously altered or modified within the scope of the present invention, and the present invention obviously covers such alterations or modifications.

1. A body composition measurement method, in which an impedance of a body of a subject is measured and information about a body composition and/or health condition of the subject is estimated based on a measurement value of an impedance, or based on the measurement value of the impedance and body specific information, wherein:
an entire human body is represented by a model in which the human body is divided into plural body parts, where the impedance of each body part can be approximately represented by a model composed of impedances respectively corresponding to at least fatty tissue, muscular tissue and osseous tissue connected in parallel, and component ratios of the tissues and electrical characteristics of the whole component tissues and each tissue can be regarded as fixed,
an alternating current is supplied between two current-carrying electrodes attached to a surface of the body at positions located outer than both ends of a target body part selected from the plural body parts so that the alternating current flows through at least the target body part,
a potential difference generated by the alternating current between both ends of the target body part is measured with two measuring electrodes attached to the surface of the body in the vicinity of the aforementioned ends or at positions lying not on a path of the alternating current but on paths each extending from the aforementioned ends, and
the impedance corresponding to the target body part is derived from a measurement value of the potential difference and a current value, and information relating to the body composition and/or health condition of the target body part or the entire body of the subject is estimated from the value of the impedance, or from the value and the body specific information.

2. The body composition measurement method according to claim 1, wherein the body parts are parts of the body, each of which can be approximately modeled as a column having a certain length and composed of tissues with their cross-sectional area ratios almost fixed.

3. The body composition measurement method according to claim 2, wherein the body parts are right and left arms and legs, and a trunk.

4. The body composition measurement method according to claim 2, wherein the body parts are right and left forearms, upper arms, crura and thighs, and a trunk.

5. The body composition measurement method according to claim 3 or 4, wherein the body parts further includes right and left wrist areas and ankle areas.

6. The body composition measurement method according to claim 2, wherein the body parts include at least a part of a forearm close to a wrist area or a part of a crus close to an ankle area on either right or left.

7. The body composition measurement method according to one of claims 3-6, wherein the trunk is represented by a model including five impedance elements: central part of the trunk; right and left shoulders connecting upper ends of right and left arms and an upper end of the central part of the trunk; and right and left groins connecting upper ends of right and left legs and a lower end of the central part of the trunk, and the impedance of the right or left shoulder, or right or left groin, is estimated from at least an impedance corresponding to one of the plural body parts.

8. The body composition measurement method according to one of claims 3-7, wherein the information relating to the body composition and/or health condition of the subject is estimated from the impedances corresponding to the trunk and at least one of the plural body parts other than the trunk.

9. The body composition measurement method according to one of claims 1-8, which uses an estimation formula created based on a result of a measurement of the impedance for the entire body and/or each body part of each of plural pretest subjects, and based on a body composition criteria information of the entire body and/or each body part of each of the pretest subjects measured and collected with an apparatus capable of acquiring cross-sectional images, or created by further including additional body specific information of the pretest subjects, in order to estimate the information relating to the body composition and/or health condition based on the measurement value of the impedance of each body part of the subject, or based on the aforementioned value and the body specific information.

10. The body composition measurement method according to one of claims 1-9, wherein body composition information is derived from at least an significant measurement value of the impedances corresponding to all the body parts included in the target body parts, or from the significant measurement value and the body specific information.

11. A body composition measurement apparatus, including a measuring means for measuring an impedance of a body of a subject, and an estimating means for estimating information relating to a body composition and/or health condition of the subject based on a measurement value of the impedance or based on the measurement value and body specific information, wherein:
an entire human body is represented by a model in which the human body is divided into plural body parts, where the impedance of each body part can be approximately represented by a model composed of impedances respectively corresponding to at least fatty tissue, muscular tissue and osseous tissue connected in parallel, and component ratios of the tissues and electrical characteristics of the whole component tissues and each tissue can be regarded as fixed,
and the measuring means include:
a) a current-generating means for generating an alternating current at a preset frequency;
b) at least two current-carrying electrodes to be attached to a surface of the body at positions outer than both ends of a target body part selected from the plural body parts to supply the alternating current through at least the target body part;
c) a voltage-measuring means, including at least two measuring electrodes to be attached to the surface of the body in the vicinity of the aforementioned ends of the target body part or at positions lying not on a path of the alternating current but on paths each extending from the aforementioned ends, for measuring a potential difference generated between the aforementioned ends of the target body part by the alternating current supplied from the current-carrying electrodes; and
d) a calculating means for calculating the impedance corresponding to the target body part from a measurement value of the potential difference and a current value of the alternating current,
and the estimating means estimates the information relating to the body composition and/or health condition of the target body part or the entire body of the subject based on a value of the impedance calculated by the calculating means, or based on the value and the body specific information.

12. The body composition measurement apparatus according to claim 11, wherein attachment points for the measuring electrodes include at least four points in the vicinity of right and left wrists and right and left ankles.

13. The body composition measurement apparatus according to claim 12, wherein the attachment points for the measuring electrodes further include at least one of four points in the vicinity of right and left elbows and right and left knees.

14. The body composition measurement apparatus according to claim 13, wherein the attachment points for the measuring electrodes further include at least one of four points located at palms or backs of right and left hands, and at soles or insteps of right and left feet.

15. The body composition measurement apparatus according to claim 13 or 14, wherein the attachment points for the measuring electrodes further include at least one of four points in the vicinity of roots of right and left arms and roots of right and left legs.

16. The body composition measurement apparatus according to claim 11, wherein the attachment points for the measuring electrodes further include a part of an arm close to a wrist area or a part of a crus close to an ankle area.

17. The body composition measurement apparatus according to one of claims 12-16, wherein the attachment points for the current-carrying electrodes are four points located between the wrist and fingertips of right and left hands, and between the ankle and fingertips of right and left feet.

18. The body composition measurement apparatus according to claim 17, wherein the attachment points for the current-carrying electrodes include a finger or fingers of the hand or the feet, and the current-carrying electrodes are designed to be fixedly held between fingers or around a finger.

19. The body composition measurement apparatus according to one of claims 11-18, wherein the body is divided into five segments corresponding to right and left arms, right and left legs and a trunk, where the segments corresponding to the arms and the legs are each modeled as one impedance element, whereas the trunk is represented by a model including five impedance elements: central part of the trunk; right and left shoulders connecting upper ends of the right and left arms and an upper end of the central part of the trunk; and right and left groins connecting upper ends of the right and left legs and a lower end of the central part of the trunk, and the estimating means estimates the impedances of the right and left shoulders and the right and left groins based on the impedance of at least one of the plural body parts of the subject.

20. The body composition measurement apparatus according to claim 17 or 18, wherein the apparatus comprises four current-carrying electrodes, four measuring electrodes and a current-carrying electrode selecting means for selectively supplying the alternating current between two of the four current-carrying electrodes, the voltage-measuring means selects two of the four measuring electrodes and measures the potential difference between the two measuring electrodes, the measuring electrodes are attached to the body at four points in the vicinity of the right and left wrist and the right and left ankles, or in the vicinity of the right and left elbows and the right and left knees, and the current-carrying electrodes are attached to the body at four positions located between the wrist and fingertips of the right and left hands, and between the ankle and fingertips of the right and left feet.

21. The body composition measurement apparatus according to claim 20, wherein the attachment positions of the four measuring electrodes are changed from four points in the vicinity of the right and left wrists and the right and left ankles to four points in the vicinity of the right and left elbows and the right and left knees, or vice versa, and the impedance of a predetermined body part is measured for each arrangement of the attachment positions.

22. The body composition measurement apparatus according to one of claims 11-20, which sequentially measures the impedances of desired body parts while changing the attachment positions of the measuring electrodes within the attachment points.

23. The body composition measurement apparatus according to claim 21 or 22, comprising a work-guiding means for indicating the attachment positions for the electrodes on the body of the subject, using at least one of image information, character information and voice information.

24. The body composition measurement apparatus according to claim 23, wherein the work-guiding means includes an image-displaying means for showing markers on a figurative model of the human body to indicate the positions at which the measuring electrodes should be attached, and a display-controlling means for controlling the image-displaying means so that the markers are moved to new positions at which the measuring electrodes should be attached next after the completion of the measurement performed with the measuring electrodes attached at preset positions.

25. The body composition measurement apparatus according to claim 23, wherein the display-controlling means is constructed to control the image-displaying means so that the body part undergoing the measurement is visually distinguishable from other body parts.

26. The body composition measurement apparatus according to one of claims 11-25, wherein the estimating means uses an estimation formula created based on a result of the measurement of the impedance for the entire body and/or each body part of each of plural pretest subjects, and based on body composition criteria information of the entire body and/or each body part of each of the pretest subjects measured and collected with an apparatus capable of acquiring cross-sectional images, or further including additional body specific information of the pretest subjects, in order to estimate the information relating to the body composition and/or health condition based on the measurement value of the impedance of each body part of the subject, or based on the measurement value and the body specific information.

27. The body composition measurement apparatus according to one of claims 11-26, wherein the body specific information includes a height, and the estimating means estimates lengths of limbs or lengths of smaller body parts from the body specific information including at least the height of the subject, obtains body composition information about the limbs or the smaller body parts referring to the estimated value, and visually displays the information.

28. The body composition measurement apparatus according to claim 27, wherein the estimated values of the lengths of the limbs or the lengths of the smaller body parts derived from the information including at least the height of the subject can be externally modified.

29. The body composition measurement apparatus according to one of claims 11-28, wherein the body specific information includes a height and a weight, and the apparatus further comprises an image-displaying means for displaying information indicating an external body type calculated from the height and the weight, with information indicating an internal body type determined based on the body composition information estimated from the measurement value of the impedance.

30. The body composition measurement apparatus according to one of claims 11-29, further comprising an image-displaying means that uses a circular chart to display body composition component ratios based on the body composition information estimated from the measurement values of the impedance, where, for each of plural different body composition types, component ratios are concentrically displayed within each of ranges formed by sectioning the circular chart in radial directions.

31. The body composition measurement apparatus according to one of claims 11-30, further comprising an image-displaying means, which includes a setting display area for entering and setting the body specific information and a result display area for showing a result of the measurement, both located within a single screen.

32. The body composition measurement apparatus according to one of claims 11-31, wherein the information relating to the body composition and/or health condition includes a balance of right and left sides of the body and a balance of the measured segments, or a balance of upper and lower halves of the body and a balance of the measured segments, with respect to a muscle mass and/or bone mass of four limbs.

33. The body composition measurement apparatus according to one of claims 11-32, wherein the information relating to the body composition and/or health condition includes an ADL index for measuring the ability for activity of daily living.

34. The body composition measurement apparatus according to one of claims 11-32, wherein the information relating to the body composition and/or health condition includes a bone density of the subject.

35. The body composition measurement apparatus according to claim 34, which estimates the bone density based on the impedance of a part in the vicinity of the wrist or ankle.

36. The body composition measurement apparatus according to claim 35, wherein an estimation process of the bone density includes a step of correcting the bone density using the impedance of an arm or leg, or using information relating to its size.

37. The body composition measurement apparatus according to one of claims 11-32, wherein the information relating to the body composition and/or health condition calculated by the body composition measurement apparatus includes basal metabolic rate or energy metabolism of the subject.

38. The body composition measurement apparatus according to claim 38, which estimates the basal metabolic rate or energy metabolism mainly based on a muscle mass of a leg, or of a thigh and a crus.

39. The body composition measurement apparatus according to claim 38, which estimates the basal metabolic rate or energy metabolism taking account of a fat mass of the entire body or a part of the body.

40. The body composition measurement apparatus according to one of claims 11-40, wherein the calculating means and the estimating means are embodied by running a predetermined control program on a multi-purpose personal computer, the current-generating means and the voltage-measuring means exclusive of the measuring electrodes are enclosed in a main unit contained in a casing with the personal computer, and the main unit and the personal computer are capable of communicating with each other

41. The body composition measurement apparatus according to claim 40, wherein the current-carrying electrodes and the measuring electrodes may be connected to the main unit via cables.

42. The body composition measurement apparatus according to one of claims 11-41, a key operation on a keyboard of a personal computer is associated with a clicking operation on a button displayed on a screen so that a selecting or commanding operation that requires a user input during the measurement can be similarly performed by either of the key operation and the clicking operation.

43. The body composition measurement apparatus according to one of claims 11-42, which measures at least two of the plural body parts and uses the difference or ratio of the body composition information of these body parts estimated from the two measurement values of the impedances of the two body parts or based on the measurement values and the body specific information, to improve the accuracy of estimating the information relating to the body composition of the entire body or a part of the body of the subject.

44. The body composition measurement apparatus according to claim 43, wherein said two body parts are serially connected within the body.

45. The body composition measurement apparatus according to claim 43 or 44, which at least improves an accuracy of estimating component ratios of at least a muscular tissue and an osseous tissue.

46. A body composition measurement apparatus, comprising:
a) a measuring means for measuring an impedance of almost an entire body or a part of the body of a subject; and
b) an estimating means for estimating an ADL index for measuring an ability for activities of daily living of the subject based on a measurement value of the impedance, or based on the measurement value and body specific information.

47. The body composition measurement apparatus according to claim 46, wherein the estimating means estimates a force that a muscle of a predetermined body part essential for the activities of daily living can exhibit, based on the measurement value of the impedance, or based on the measurement value and the body specific information, and determines the force, or a value derived from the force, as the ADL index.

48. The body composition measurement apparatus according to claim 48, wherein the estimating means estimates a muscle mass of a predetermined part of the body that is essential for the activities of daily living, based on the measurement value of the impedance, or based on the measurement value and the body specific information, and further estimates, from the muscle mass, the force that the muscle concerned can exhibit.

49. The body composition measurement apparatus according to claim 47 or 48, wherein the muscle of the predetermined part of the body is a muscle included in a thigh or crus of the subject, the measuring means measures the impedance of at least a part of a lower half of the body of the subject, and the estimating means estimates a mass or muscle force of the muscle included in the thigh or crus based on the measurement value of the impedance, or based on the measurement value and the body specific information.

50. The body composition measurement apparatus according to claim 49, wherein the muscle of the predetermined part of the body includes at least a quadriceps.

51. The body composition measurement apparatus according to claim 50, which estimates the muscle masses of right and left quadricepses and provides life-improving advice based on the muscle masses and their right-and-left balance.

52. A body composition measurement apparatus, comprising:
a) plural current-carrying electrodes and plural measuring electrodes to be attached to a body of a subject, to measure an impedance of a target part composed of one body part or two or more body parts connected in series, based on a model in which an entire human body is separated into plural body parts, where the impedance of each body part can be represented by a model composed of impedances respectively corresponding to at least fatty tissue, muscular tissue and osseous tissue connected in parallel, and component ratios of the tissues and electrical characteristics of the whole component tissues and each tissue can be regarded as fixed;
b) a current-supplying means for supplying an alternating current at a preset frequency via the current-carrying electrodes through at least the target part;
c) a voltage-measuring means for measuring a voltage generated by the alternating current between both ends of the target part; and
d) a calculating means for calculating the impedance corresponding to the target part from the voltage measured and a current value of the alternating current, and for estimating information relating to a body composition or health condition of the target part or entire body of the subject from a value of the impedance, or from the value and body specific information, using an estimation formula created based on a result of a measurement of the impedance for the entire body and/or each body part of each of plural pretest subjects, and based on body composition criteria information of the entire body and/or each body part of each of the pretest subjects measured and collected with an apparatus capable of acquiring cross-sectional images, or created by further adding body specific information of the pretest subjects.

53. The body composition measurement apparatus according to claim 52, wherein the plural measuring electrodes are attached to at least two points in the vicinity of right and left wrists, right and left elbows, right and left knees, palms and backs of right and left hands, and soles or insteps of right and left feet.

54. The body composition measurement apparatus according to claim 53, wherein the attachment points for the measuring electrodes further include at least one of four points in the vicinity of right and left elbows and right and left knees.

55. The body composition measurement apparatus according to claim 53, wherein the attachment points for the measuring electrodes further include at least one of four points in the vicinity of the right and left elbows and right and left knees.

56. The body composition measurement apparatus according to claim 54 or 55, wherein the attachment points for the measuring electrodes further include at least one of four points located at the palms or backs of right and left hands, and the soles or insteps of the right and left feet.

57. The body composition measurement apparatus according to claim 54, wherein the attachment points for the measuring electrodes further include at least one point located between the wrist and the elbow or between the ankle and the knee.

58. The body composition measurement apparatus according to one of claims 52-57, wherein the plural current-carrying electrodes include at least four electrodes to be attached to four points located between the wrists and fingertips of right and left hands, and between the ankles and fingertips of right and left feet.

59. The body composition measurement apparatus according to one of claims 52-57, wherein two measuring electrodes to be attached to a point in the vicinity of the wrist and a point between the wrist and the elbow are formed on one face of the same sheet-like member, with a predetermined distance between them, and the sheet-like member is affixed to a skin of the subject to perform the measurement.

60. The body composition measurement apparatus according to claim 59, wherein the current-carrying electrode is also formed on said face of the sheet-like member.

61. The body composition measurement apparatus according to one of claims 52-58, wherein the current-carrying electrodes and the measuring electrodes are attachable to and detachable from a skin, and the electrodes are connected to the current-supplying means and the voltage-measuring means via cables.

62. The body composition measurement apparatus according to one of claims 52-54, further comprising a measuring platform for the subject to step on, and grips for the subject to hold with both hands, where the measuring platform has, on its top, the current-carrying electrodes to contact the finger-side parts of the soles and the measuring electrodes to contact the ankle-side part of the soles, and each grip has the measuring electrode to contact a point in the vicinity of the wrist and the current-carrying electrode to contact a predetermined point in the hand.

63. The body composition measurement apparatus according to one of claims 52-54, further comprising a measuring platform for the subject to step on and a pair of armrests for supporting the arms of the subject standing on the measuring platform with both arms stretched substantially forward, where the measuring platform has, on its top, the current-carrying electrodes to contact the finger-side part of the soles and the measuring electrodes to contact the ankle-side part of the soles, and each armrest has, on its top, the measuring electrode to contact a point in the vicinity of the wrist and the current-carrying electrode to contact a predetermined point in the hand.

64. The body composition measurement apparatus according to one of claims 52-54, further comprising a measuring platform for the subject to step on, a chair for the subject to sit down on with the feet placed on the measuring platform, and armrests for the subject to rest the forearms on while sitting on the chair, where the measuring platform has, on its top, the current-carrying electrodes to contact the finger-side part of the soles and the measuring electrodes to contact the ankle-side part of the soles, and each armrest has, on its top, the measuring electrode to contact a point in the vicinity of the wrist and the current-carrying electrode to contact a predetermined point in the hand.

65. The body composition measurement apparatus according to claim 63 or 64, wherein the armrests has, on their top, a pair of grips to be held with both hands, each grip having the current-carrying electrode.

66. The body composition measurement apparatus according to claim 65, wherein the grip is a substantially columnar body having the current-carrying electrode in an upper part and the measuring electrode in a lower part with a predetermined gap from the current-carrying electrode.

67. The body composition measurement apparatus according to one of claims 63-66, wherein the armrest has, on its top, with a measuring electrode to contact a point in the vicinity of the elbow.

68. The body composition measurement apparatus according to one of claims 63-67, comprising an ankle-measuring part having a measuring electrode to contact the ankle of the subject is further provided.

69. The body composition measurement apparatus according to one of claims 63-68, comprising a knee-measuring part having a measuring electrode to contact an inside or back of the knee of the subject.

70. The body composition measurement apparatus according to claim 64, comprising a measuring electrode to contact a back of the knee, located in the vicinity of a front corner of the seat of the chair.

71. The body composition measurement apparatus according to claim 63, comprising a weighing means for measuring a weight of the subject on the measuring platform and a height-measuring means for measuring a height of the subject in a standing position, and using the weight and the height as the body specific information.
